(19) United States
(12) Patent Application Publication
(10) Pub. No.: US 2022/0049252 A1
(43) Pub. Date: Feb. 17, 2022

(54) CHEMICALLY-MODIFIED RNAi CONSTRUCTS AND USES THEREOF

(71) Applicant: AMGEN INC., Thousand Oaks, CA (US)

(72) Inventors: Justin K. MURRAY, Moorpark, CA (US); Bin WU, Thousand Oaks, CA (US); Yuan CHENG, Newbury Park, CA (US); Bradley J. HERBERICH, Newbury Park, CA (US)

(73) Assignee: AMGEN INC., Thousand Oaks, CA (US)

(21) Appl. No.: 17/312,383

(22) PCT Filed: Dec. 9, 2019
(86) PCT No.: PCT/US2019/065294

§ 371 Date: Jun. 9, 2021

Related U.S. Application Data
(60) Provisional application No. 62/777,677, filed on Dec. 10, 2018.

(51) Int. Cl. C12N 15/113; A61K 31/713

(52) U.S. Cl. CPC C12N 15/113; A61K 31/713; C12N 2310/14; C12N 2310/322; C12N 2310/321; C12N 2310/3231; C12N 2310/3533; C12N 2310/344; C12N 2310/345; C12N 2310/346; C12N 2310/3521; C12N 2310/3523; C12N 2310/3525; C12N 2310/351

(57) ABSTRACT

The present invention relates to chemically-modified RNAi constructs for reducing expression of a target gene. In particular, the invention relates to specific patterns of modified nucleotides to be incorporated into RNAi constructs to improve in vivo stability and efficacy. Also described are pharmaceutical compositions comprising the chemically-modified RNAi constructs and methods of inhibiting target gene expression in vivo by administering the chemically-modified RNAi constructs, for example, to treat or ameliorate various disease conditions.

### CROSS-REFERENCE TO RELATED APPLICATIONS

This application claims the benefit of U.S. Provisional Application No. 62/777,677, filed Dec. 10, 2018, which is hereby incorporated by reference in its entirety.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Dec. 9, 2019, is named A-2327-WO-PCT_SeqList_ST25 and is 24.7 kilobytes in size.

### FIELD OF THE INVENTION

The present invention relates to chemically-modified RNAi constructs for reducing expression of a target gene in vivo. Specifically, the invention relates to specific patterns of modified nucleotides that impart improved efficacy and stability of RNAi constructs in vivo. Such RNAi constructs are useful for inhibiting target gene expression for therapeutic purposes.

### BACKGROUND OF THE INVENTION

RNA interference (RNAi) is a post-transcriptional gene silencing mechanism found in almost all phyla and believed to be an evolutionary-conserved cellular defense mechanism (Fire et al., Nature, Vol. 391; 806-811, 1998; Fire et al., Trends Genet, Vol. 15: 358-363, 1999; and Hamilton and Baulcombe, Science, Vol. 286, 950-952, 1999). Physiologically, the RNAi mechanism is initiated by Dicer enzyme-mediated generation of duplexes of 18-25 base pairs from longer non-coding RNAs. These short RNA molecules are loaded into the RNA-induced silencing complex (RISC), where the sense strand or passenger strand is discarded, and the antisense strand or guide strand hybridizes to a completely or partially complementary mRNA sequence (Nakanishi, Wiley Interdiscip. Rev. RNA, Vol. 7: 637-660, 2016). Silencing of the mRNA is then induced via Ago2-mediated degradation or translational repression (Bobbin and Rossi, Annu. Rev. Pharmacol. Toxicol., Vol. 56:103-122, 2016).

Advancements in RNAi technology and delivery methodology have led to a growing number of positive outcomes with RNAi-based therapies. Such therapies represent a promising class of therapeutics, particularly against targets that have been deemed “undruggable” by small molecule or biologic modalities. Although much progress has been made to overcome the inherent metabolic liabilities of natural RNA through the development of chemical modifications and improved delivery methods, there remains a need in the art for RNAi agents with enhanced in vivo efficacy and stability suitable for administration for therapeutic purposes.

### SUMMARY OF THE INVENTION

The present invention is based, in part, on the design of chemical modification patterns for RNAi constructs that improve the potency and/or duration of gene silencing activity of the constructs in vivo. The modification patterns described herein can be universally applied to a variety of RNAi constructs having different sequences and targets. The RNAi constructs are useful for inhibiting target gene expression in vivo, for example for therapeutic purposes.

Accordingly, the present invention provides RNAi constructs that inhibit expression of a target gene sequence, wherein the RNAi constructs comprise a sense strand and an antisense strand, wherein the antisense strand comprises a sequence that is complementary to the target gene sequence and the sense strand comprises a sequence that is sufficiently complementary to the sequence of the antisense strand to form a duplex region, and wherein the RNAi constructs comprise a structure represented by one of the formulas described herein. In certain embodiments, the RNAi constructs of the invention have a chemical modification pattern selected from one of the patterns designated as P1 to P30 as described herein.

In some embodiments, the RNAi construct comprises a structure represented by Formula (A):

(A)
5′-(N_{A})ₓ N_{L} N_{L} N_{L} N_{L} N_{L} N_{L} N_{F} N_{L} N_{F} N_{F} N_{F} N_{F} N_{L} N_{L}
N_{M} N_{L} N_{M} N_{L} N_{T} (n)_{y}-3′
3′-(N_{B})_{z} N_{L} N_{L} N_{L} N_{L} N_{L} N_{F} N_{L} N_{M} N_{L} N_{M} N_{L} N_{L} N_{F} N_{M}
N_{L} N_{M} N_{L} N_{F} N_{L}-5′

In Formula (A), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a bicyclic nucleic acid (BNA), and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{B} nucleotides can be complementary to N_{A} nucleotides when present in the sense strand or can be overhang nucleotides that do not base pair with nucleotides in the sense strand.

In some embodiments, the RNAi construct comprises a sense strand of 19-23 nucleotides in length and an antisense strand of 19-23 nucleotides in length, wherein the sequences of the antisense stand and the sense strand are sufficiently complementary to each other to form a duplex region of 19-21 base pairs, wherein: nucleotides at positions 2, 7, and 14 in the antisense strand (counting from the 5′ end) are 2′-fluoro modified nucleotides; nucleotides in the sense strand at positions paired with positions 8 to 11 and 13 in the antisense strand (counting from the 5′ end) are 2′-fluoro modified nucleotides; and neither the sense strand nor the antisense strand each have more than 7 total 2′-fluoro modified nucleotides. The RNAi construct can have a nucleotide overhang at one or both of the 3′ ends of the sense strand and the antisense strand. In certain embodiments, the RNAi construct has a nucleotide overhang at the 3′ end of the antisense strand and a blunt end at the 5′ end of the antisense strand.

In other embodiments of the invention, the RNAi construct comprises a structure represented by Formula (D):

(D)
5′-(N_{A})ₓ N_{L} N_{L} N_{L} N_{L} N_{M} N_{L} N_{F} N_{F} N_{F} N_{F} N_{L} N_{L} N_{L} N_{L}
N_{L} N_{L} N_{L} N_{L} N_{T}(n)_{y}-3′
3′-(N_{B})_{z} N_{L} N_{L} N_{L} N_{M} N_{L} N_{F} N_{L} N_{M} N_{L} N_{L} N_{M} N_{M} N_{M} N_{M}
N_{L} N_{M} N_{L} N_{F} N_{L}-5′

In Formula (D), the top strand listed in the 5′ to 3′ direction is the sense strand and the bottom strand listed in the 3′ to 5′ direction is the antisense strand; each N_{F} represents a 2′-fluoro modified nucleotide; each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and NT represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. X can be an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{A} nucleotides can be complementary to nucleotides in the antisense strand. Y can be an integer from 0 to 4, provided that when y is 1, 2, 3, or 4, one or more n nucleotides are modified or unmodified overhang nucleotides that do not base pair with nucleotides in the antisense strand. Z can be an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide. One or more of the N_{B} nucleotides can be complementary to N_{A} nucleotides when present in the sense strand or can be overhang nucleotides that do not base pair with nucleotides in the sense strand.

In some embodiments of the invention, the RNAi construct comprises a sense strand of 19-23 nucleotides in length and an antisense strand of 19-23 nucleotides in length, wherein the sequences of the antisense stand and the sense strand are sufficiently complementary to each other to form a duplex region of 19-21 base pairs, wherein: nucleotides at positions 2, 14, and 16 in the antisense strand (counting from the 5′ end) are 2′-fluoro modified nucleotides; nucleotides in the sense strand at positions paired with positions 10 to 13 in the antisense strand (counting from the 5′ end) are 2′-fluoro modified nucleotides; and neither the sense strand nor the antisense strand each have more than 7 total 2′-fluoro modified nucleotides. The RNAi construct can have a nucleotide overhang at the 3′ end of the antisense strand and a blunt end at the 5′ end of the antisense strand. Alternatively, the RNAi construct can have a nucleotide overhang at both of the 3′ ends of the sense strand and the antisense strand.

The RNAi constructs of the invention can comprise at least one backbone modification, such as a modified internucleotide or internucleoside linkage. In certain embodiments, the RNAi constructs described herein comprise at least one phosphorothioate internucleotide linkage. In particular embodiments, the phosphorothioate internucleotide linkages may be positioned at the 3′ or 5′ ends of the sense and/or antisense strands.

The RNAi constructs may further comprise a ligand to facilitate delivery or uptake of the RNAi constructs to specific tissues or cells, such as liver cells. In some embodiments, the ligand targets delivery of the RNAi constructs to hepatocytes. In these and other embodiments, the ligand may comprise galactose, galactosamine, or N-acetyl-galactosamine (GalNAc). In certain embodiments, the ligand comprises a multivalent galactose or multivalent GalNAc moiety, such as a trivalent or tetravalent galactose or GalNAc moiety. The ligand may be covalently attached to the 5′ or 3′ end of the sense strand of the RNAi construct, optionally through a linker. In some embodiments, the RNAi constructs comprise a ligand and linker having a structure according to any of Formulas Ito IX described herein. In one embodiment, the RNAi constructs comprise a ligand and linker having a structure according to Formula VI. In another embodiment, the RNAi constructs comprise a ligand and linker having a structure according to Formula VII. In yet another embodiment, the RNAi constructs comprise a ligand and linker having a structure according to Formula IX.

The present invention also provides pharmaceutical compositions comprising any of the RNAi constructs described herein and a pharmaceutically acceptable carrier, excipient, or diluent. Such pharmaceutical compositions are particularly useful for reducing or inhibiting expression of a target gene in the cells (e.g. liver cells) of a subject, particularly when overexpression of the target gene product in the subject is associated with a pathological phenotype.

The present invention includes methods for reducing or inhibiting expression of a target gene in a cell, tissue, or subject. In one embodiment, the methods comprise contacting the cell or tissue with any one of the RNAi constructs described herein. The cell or tissue may be in vitro or in vivo. In another embodiment, the methods comprise administering any one of the RNAi constructs described herein to a subject. The RNAi constructs can be administered to the subject parenterally (e.g. intravenously or subcutaneously).

### BRIEF DESCRIPTION OF THE DRAWINGS

FIG. 1 shows several representative embodiments of chemical modification patterns for RNAi constructs. In each of the schematics, the top strand represents the sense strand in the 5′ to 3′ direction and the bottom strand represents the antisense strand in the 3′ to 5′ direction. Solid black circles represent 2′-O-methyl (2′-OMe) modified nucleotides, striped circles represent 2′-fluoro (2′-F) modified nucleotides, and white circles represent inverted abasic nucleotides (invAb) or inverted deoxyribonucleotides (invdN). Light gray lines connecting the circles represent phosphodiester linkages, whereas black lines connecting the circles represent phosphorothioate linkages. The black boxes denote the putative Ago2 cleavage sites within the RNAi constructs.

FIG. 2 is a bar graph of human PNPLA3 variant expression levels in livers of mice injected with an AAV encoding the human PNPLA3 variant and treated with 5 mg/kg subcutaneous injections of the indicated RNAi construct having the P1 or CM1 chemical modification pattern. Human PNPLA3 expression was measured by qPCR and is reported as expression levels relative to vehicle-treated animals. Expression levels are shown at day 8 after RNAi construct administration.

FIG. 3 is a bar graph of human PNPLA3 variant expression levels in livers of mice injected with an AAV encoding the human PNPLA3 variant and treated with 5 mg/kg subcutaneous injections of the indicated RNAi construct having the P1, P2, P3, or P4 chemical modification patterns. Human PNPLA3 expression was measured by qPCR and is reported as expression levels relative to vehicle-treated animals. Expression levels are shown at day 15 after RNAi construct administration.

FIGS. 4A and 4B are line graphs depicting total flux (photons per second) in mice receiving subcutaneous injections of vehicle or the indicated RNAi constructs having the P9 chemical modification pattern at a dose of 1 mg/kg (FIG. 4A) or 3 mg/kg (FIG. 4B) versus the number of weeks post-RNAi construct injection. Total flux represents the signal from a luciferase reporter, which contains sequences complementary to the sequences of the RNAi constructs, expressed by the mice. A reduction in total flux is indicative of a reduction in expression of the luciferase reporter.

FIG. 5 is a bar graph of human PNPLA3 variant expression levels in livers of mice injected with an AAV encoding the human PNPLA3 variant and treated with 3 mg/kg subcutaneous injections of the indicated RNAi constructs having the P9 (duplex nos. 7318 and 8709), CM2 (duplex no. 8103), CM3 (duplex no. 8104), or CM4 (duplex no. 8105) chemical modification patterns. Human PNPLA3 expression was measured by qPCR and is reported as expression levels relative to vehicle-treated animals. Expression levels are shown at day 28 after RNAi construct administration.

FIG. 6 is a bar graph of mouse ASGR1 expression levels in livers of mice treated with 5 mg/kg subcutaneous injections of the indicated ASGR1 RNAi constructs. Mouse ASGR1 expression was measured by qPCR and is reported as expression levels normalized by Gapdh expression levels. Expression levels are shown at day 4, day 8, and day 15 after RNAi construct or buffer (phosphate buffered saline, PBS) administration.

FIG. 7 is a line graph showing the percent change in serum Lp(a) levels relative to baseline in double transgenic mice administered 0.5 mg/kg subcutaneous injections of the indicated LPA-targeted RNAi constructs. Both RNAi constructs had the same sequence and differed only in the pattern of chemical modifications; duplex no. 3632 had the CM1 modification pattern and duplex no. 3635 had the P1 modification pattern. The percent change in Lp(a) serum levels is shown at day 14 (D14) and day 28 (D28) following the single subcutaneous injection of the RNAi constructs.

### DETAILED DESCRIPTION

The present invention is based, in part, on the design of chemical modification patterns for RNAi constructs that produce potent and durable knockdown of target gene expression in vivo across a variety of sequences and targets. The chemically-modified RNAi constructs described herein were shown to have improved potency and/or duration in gene silencing activity in vivo as compared to previously-described therapeutic RNAi agents having alternative chemical modification patterns. The modified RNAi constructs of the invention are useful for inhibiting target gene expression in vivo, e.g., for treating or ameliorating various disease conditions. Accordingly, the present invention provides RNAi constructs that inhibit expression of a target gene sequence.

As used herein, the term “RNAi construct” refers to an agent comprising an RNA molecule that is capable of downregulating expression of a target gene via an RNA interference mechanism when introduced into a cell. RNA interference is the process by which a nucleic acid molecule induces the cleavage and degradation of a target RNA molecule (e.g. messenger RNA or mRNA molecule) in a sequence-specific manner, e.g. through an RNA-induced silencing complex (RISC) pathway. In some embodiments, the RNAi construct comprises a double-stranded RNA molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target sequence (e.g. target mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, about 85%, about 90%, about 95%, about 96%, about 97%, about 98%, or about 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be considered to be fully complementary as the term is used herein.

In some embodiments, a region of the antisense strand comprises a sequence that is fully complementary to a region of the target gene sequence (e.g. target mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). Thus, in some embodiments, the RNAi constructs of the invention comprise an siRNA.

In other embodiments, the sense strand and the antisense strand that hybridize to form a duplex region may be part of a single RNA molecule, i.e. the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Such RNA molecules with at least partially self-complementary regions are referred to as “short hairpin RNAs” (shRNAs). In certain embodiments, the RNAi constructs of the invention comprise a shRNA. The length of a single, at least partially self-complementary RNA molecule can be from about 40 nucleotides to about 100 nucleotides, from about 45 nucleotides to about 85 nucleotides, or from about 50 nucleotides to about 60 nucleotides and comprise a duplex region and loop region each having the lengths recited herein.

The RNAi constructs of the invention comprise a sense strand and an antisense strand, wherein the antisense strand comprises a region having a sequence that is substantially or fully complementary to a target gene sequence. A target gene sequence generally refers to a nucleic acid sequence that comprises a partial or complete coding sequence for a polypeptide. The target gene sequence may also include a non-coding region, such as the 5′ or 3′ untranslated region (UTR). In certain embodiments, the target gene sequence is a messenger RNA (mRNA) sequence. An mRNA sequence refers to any messenger RNA sequence, including splice variants, encoding a protein, protein variants, or isoforms from any species (e.g. mouse, rat, non-human primate, human). In one embodiment, the target gene sequence is an mRNA sequence encoding a human protein. A target gene sequence can also be an RNA sequence other than an mRNA sequence, such as a tRNA sequence, microRNA sequence, or viral RNA sequence.

A region of the antisense strand of the RNAi construct can be substantially complementary or fully complementary to at least 15 consecutive nucleotides of a target gene sequence. In some embodiments, the target region of the gene sequence to which the antisense strand comprises a region of complementarity can range from about 15 to about 30 consecutive nucleotides, from about 16 to about 28 consecutive nucleotides, from about 18 to about 26 consecutive nucleotides, from about 17 to about 24 consecutive nucleotides, from about 19 to about 30 consecutive nucleotides, from about 19 to about 25 consecutive nucleotides, from about 19 to about 23 consecutive nucleotides, or from about 19 to about 21 consecutive nucleotides.

The sense strand of the RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the RNAi construct should be of sufficient length to allow the RNAi construct to enter the RNA interference pathway, e.g. by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In one embodiment, the duplex region is about 17 to about 24 base pairs in length. In another embodiment, the duplex region is about 19 to about 21 base pairs in length. In certain embodiments, the duplex region is about 19 base pairs in length. In other embodiments, the duplex region is about 21 base pairs in length.

For embodiments in which the sense strand and antisense strand are two separate molecules (e.g. RNAi construct comprises a siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. In certain other embodiments, the nucleotide overhang comprises a single nucleotide.

The nucleotides in the overhang can be ribonucleotides or modified nucleotides as described herein. In some embodiments, the nucleotides in the overhang are 2′-modified nucleotides (e.g. 2′-fluoro modified nucleotides, 2′-O-methyl modified nucleotides), deoxyribonucleotides, inverted nucleotides (e.g. inverted abasic nucleotides, inverted deoxyribonucleotides), or combinations thereof. For instance, in one embodiment, the nucleotides in the overhang are deoxyribonucleotides, e.g. deoxythymidine. In another embodiment, the nucleotides in the overhang are 2′-O-methyl modified nucleotides, 2′-fluoro modified nucleotides, 2′-methoxyethyl modified nucleotides, or combinations thereof. In other embodiments, the overhang comprises a 5′-uridine-uridine-3′ (5′-UU-3′) dinucleotide. In such embodiments, the UU dinucleotide may comprise ribonucleotides or modified nucleotides, e.g. 2′-modified nucleotides. In other embodiments, the overhang comprises a 5′-deoxythymidine-deoxythymidine-3′ (5′-dTdT-3′) dinucleotide. When a nucleotide overhang is present in the antisense strand, the nucleotides in the overhang can be complementary to the target gene sequence, form a mismatch with the target gene sequence, or comprise some other sequence (e.g. polypyrimidine or polypurine sequence, such as UU, TT, AA, GG, etc.).

The nucleotide overhang can be at the 5′ end or 3′ end of one or both strands. For example, in one embodiment, the RNAi construct comprises a nucleotide overhang at the 5′ end and the 3′ end of the antisense strand. In another embodiment, the RNAi construct comprises a nucleotide overhang at the 5′ end and the 3′ end of the sense strand. In some embodiments, the RNAi construct comprises a nucleotide overhang at the 5′ end of the sense strand and the 5′ end of the antisense strand. In other embodiments, the RNAi construct comprises a nucleotide overhang at the 3′ end of the sense strand and the 3′ end of the antisense strand.

The RNAi constructs may comprise a nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the RNAi construct comprises a nucleotide overhang at the 3′ end of the sense strand and a blunt end at the 5′ end of the sense strand and 3′ end of the antisense strand. In other embodiments, the RNAi construct comprises a nucleotide overhang at the 3′ end of the antisense strand and a blunt end at the 5′ end of the antisense strand and the 3′ end of the sense strand. In certain embodiments, the RNAi construct comprises a blunt end at both ends of the double-stranded RNA molecule. In such embodiments, the sense strand and antisense strand have the same length and the duplex region is the same length as the sense and antisense strands (i.e. the molecule is double-stranded over its entire length).

The sense strand and antisense strand in the RNAi constructs of the invention can each independently be about 15 to about 30 nucleotides in length, about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In certain embodiments, the sense strand and antisense strand are each independently about 18, about 19, about 20, about 21, about 22, about 23, about 24, or about 25 nucleotides in length. In some embodiments, the sense strand and antisense strand have the same length but form a duplex region that is shorter than the strands such that the RNAi construct has two nucleotide overhangs. For instance, in one embodiment, the RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one such embodiment, the RNAi construct is blunt ended and comprises (i) a sense strand and an antisense strand, each of which is 21 nucleotides in length, and (ii) a duplex region that is 21 base pairs in length. In another such embodiment, the RNAi construct is blunt ended and comprises (i) a sense strand and an antisense strand, each of which is 23 nucleotides in length, and (ii) a duplex region that is 23 base pairs in length.

In other embodiments, the sense strand or the antisense strand is longer than the other strand and the two strands form a duplex region having a length equal to that of the shorter strand such that the RNAi construct comprises at least one nucleotide overhang. For example, in one embodiment, the RNAi construct comprises (i) a sense strand that is 19 nucleotides in length, (ii) an antisense strand that is 21 nucleotides in length, (iii) a duplex region of 19 base pairs in length, and (iv) a nucleotide overhang of 2 unpaired nucleotides at the 3′ end of the antisense strand. In another embodiment, the RNAi construct comprises (i) a sense strand that is 21 nucleotides in length, (ii) an antisense strand that is 23 nucleotides in length, (iii) a duplex region of 21 base pairs in length, and (iv) a nucleotide overhang of 2 unpaired nucleotides at the 3′ end of the antisense strand.

The RNAi constructs of the invention preferably comprise modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate. However, the RNAi constructs may comprise combinations of modified nucleotides and ribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of RNAi constructs for reducing expression of the target gene can also be enhanced by incorporation of modified nucleotides, particularly when incorporated in specific patterns as described in more detail herein.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. O—C₁-C₁₀ or O—C₁-C₁₀ substituted alkyl), 2′-O-allyl (O—CH₂CH═CH₂), 2′-C-allyl, 2′-deoxy-2′-fluoro (also referred to as 2′-F or 2′-fluoro), 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into the RNAi constructs of the invention are described in U.S. Pat. No. 9,181,551, U.S. Patent Publication No. 2016/0122761, and Deleavey and Damha, Chemistry and Biology, Vol. 19: 937-954, 2012, all of which are hereby incorporated by reference in their entireties.

In some embodiments, the RNAi constructs comprise one or more 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), deoxyribonucleotides, or combinations thereof. In certain embodiments, the RNAi constructs comprise one or more 2′-fluoro modified nucleotides, 2′-O-methyl modified nucleotides, 2′-O-methoxyethyl modified nucleotides, or combinations thereof. In certain embodiments, the RNAi constructs comprise one or more 2′-fluoro modified nucleotides, 2′-O-methyl modified nucleotides or combinations thereof.

Both the sense and antisense strands of the RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In certain embodiments, the modified nucleotides incorporated into one or both of the strands of the RNAi constructs of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the RNAi constructs of the invention include those described in Herdewijn, Antisense Nucleic Acid Drug Dev., Vol. 10: 297-310, 2000 and Peacock et al., J. Org. Chem., Vol. 76: 7295-7300, 2011, both of which are hereby incorporated by reference in their entireties. The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the sense and antisense strands of the RNAi constructs may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into the terminal ends of the sense and/or antisense strands of the RNAi constructs. In one embodiment, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-O-methyl modification, or a 2′-H (deoxy) modification. In one embodiment, the abasic nucleotide comprises a 2′-O-methyl modification. In another embodiment, the abasic nucleotide comprises a 2′-H modification (i.e. a deoxy abasic nucleotide).

The inventors have discovered that incorporation of modified nucleotides into RNAi constructs according to certain patterns results in RNAi constructs with improved gene silencing activity in vivo. For instance, in one embodiment, the RNAi construct of the invention comprises a sense strand and an antisense strand that comprise sequences that are sufficiently complementary to each other to form a duplex region of at least 15 base pairs, wherein:
nucleotides at positions 2, 7, and 14 in the antisense strand (counting from the 5′ end) are 2′-fluoro modified nucleotides;
nucleotides in the sense strand at positions paired with positions 8 to 11 and 13 in the antisense strand (counting from the 5′ end) are 2′-fluoro modified nucleotides; and
neither the sense strand nor the antisense strand each have more than 7 total 2′-fluoro modified nucleotides.

In other embodiments, the RNAi construct of the invention comprises a sense strand and an antisense strand that comprise sequences that are sufficiently complementary to each other to form a duplex region of at least 19 base pairs, wherein:
nucleotides at positions 2, 7, and 14 in the antisense strand (counting from the 5′ end) are 2′-fluoro modified nucleotides, nucleotides at positions 4, 6, 10, and 12 (counting from the 5′ end) are optionally 2′-fluoro modified nucleotides, and all other nucleotides in the antisense strand are modified nucleotides other than 2′-fluoro modified nucleotides; and
nucleotides in the sense strand at positions paired with positions 8 to 11 and 13 in the antisense strand (counting from the 5′ end) are 2′-fluoro modified nucleotides, nucleotides in the sense strand at positions paired with positions 3 and 5 in the antisense strand (counting from the 5′ end) are optionally 2′-fluoro modified nucleotides; and all other nucleotides in the sense strand are modified nucleotides other than 2′-fluoro modified nucleotides.

In such embodiments, the modified nucleotides other than 2′-fluoro modified nucleotides can be selected from 2′-O-methyl modified nucleotides, 2′-O-methoxyethyl modified nucleotides, 2′-O-alkyl modified nucleotides, 2′-O-allyl modified nucleotides, BNAs, and deoxyribonucleotides. In these and other embodiments, the terminal nucleotide at the 3′ end, the 5′ end, or both the 3′ end and the 5′ end of the sense strand can be an abasic nucleotide or a deoxyribonucleotide. In such embodiments, the abasic nucleotide or deoxyribonucleotide may be inverted—i.e. linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage.

In any of the above-described embodiments, nucleotides at positions 2, 7, 12, and 14 in the antisense strand (counting from the 5′ end) are 2′-fluoro modified nucleotides. In other embodiments, nucleotides at positions 2, 4, 7, 12, and 14 in the antisense strand (counting from the 5′ end) are 2′-fluoro modified nucleotides. In yet other embodiments, nucleotides at positions 2, 4, 6, 7, 12, and 14 in the antisense strand (counting from the 5′ end) are 2′-fluoro modified nucleotides. In still other embodiments, nucleotides at positions 2, 4, 6, 7, 10, 12, and 14 in the antisense strand (counting from the 5′ end) are 2′-fluoro modified nucleotides. In alternative embodiments, nucleotides at positions 2, 7, 10, 12, and 14 in the antisense strand (counting from the 5′ end) are 2′-fluoro modified nucleotides. In certain other embodiments, nucleotides at positions 2, 4, 7, 10, 12, and 14 in the antisense strand (counting from the 5′ end) are 2′-fluoro modified nucleotides.

In any of the above-described embodiments, nucleotides in the sense strand at positions paired with positions 3, 8 to 11, and 13 in the antisense strand (counting from the 5′ end) are 2′-fluoro modified nucleotides. In some embodiments, nucleotides in the sense strand at positions paired with positions 5, 8 to 11, and 13 in the antisense strand (counting from the 5′ end) are 2′-fluoro modified nucleotides. In other embodiments, nucleotides in the sense strand at positions paired with positions 3, 5, 8 to 11, and 13 in the antisense strand (counting from the 5′ end) are 2′-fluoro modified nucleotides.

In certain embodiments of the invention, the RNAi construct comprises a sense strand and an antisense strand, wherein the antisense strand comprises a sequence that is complementary to a target gene sequence and the sense strand comprises a sequence that is sufficiently complementary to the sequence of the antisense strand to form a duplex region, wherein the RNAi construct comprises a structure represented by Formula (A):

(A)
5′-(N_{A})ₓ N_{L} N_{L} N_{L} N_{L} N_{L} N_{L} N_{F} N_{L} N_{F} N_{F} N_{F} N_{F} N_{L} N_{L}
N_{M} N_{L} N_{M} N_{L} N_{T} (n)_{y}-3′
3′-(N_{B})_{z} N_{L} N_{L} N_{L} N_{L} N_{L} N_{F} N_{L} N_{M} N_{L} N_{M} N_{L} N_{L} N_{F} N_{M}
N_{L} N_{M} N_{L} N_{F} N_{L}-5′

wherein:

the top strand listed in the 5′ to 3′ direction is the sense strand and the bottom strand listed in the 3′ to 5′ direction is the antisense strand;

each N_{F} represents a 2′-fluoro modified nucleotide;

each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a bicyclic nucleic acid (BNA), and a deoxyribonucleotide;

each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

x is an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;

y is an integer from 0 to 4, provided that when y is 1, 2, 3, or 4, one or more n nucleotides are modified or unmodified overhang nucleotides that do not base pair with nucleotides in the antisense strand; and

z is an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

In some embodiments in which the RNAi construct comprises a structure represented by Formula (A), there is a nucleotide overhang at the 3′ end of the sense strand—i.e. y is 1, 2, 3, or 4. In one such embodiment, y is 2. In embodiments in which there is an overhang of 2 nucleotides at the 3′ end of the sense strand (i.e. y is 2), x is 0 and z is 2 or x is 1 and z is 2. In other embodiments in which the RNAi construct comprises a structure represented by Formula (A), the RNAi construct comprises a blunt end at the 3′ end of the sense strand and the 5′ end of the antisense strand (i.e. y is 0). In such embodiments where there is no nucleotide overhang at the 3′ end of the sense strand (i.e. y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (A), the N_{M} at positions 4 and 12 in the antisense strand counting from the 5′ end are each a 2′-fluoro modified nucleotide. In other embodiments, the N_{M} at positions 4, 6, and 12 in the antisense strand counting from the 5′ end are each a 2′-fluoro modified nucleotide. In yet other embodiments, the N_{M} at positions 4, 6, 10, and 12 in the antisense strand counting from the 5′ end are each a 2′-fluoro modified nucleotide. In alternative embodiments in which the RNAi construct comprises a structure represented by Formula (A), the N_{M} at positions 10 and 12 in the antisense strand counting from the 5′ end are each a 2′-fluoro modified nucleotide. In related embodiments, the N_{M} at positions 4, 10, and 12 in the antisense strand counting from the 5′ end are each a 2′-fluoro modified nucleotide. In other alternative embodiments in which the RNAi construct comprises a structure represented by Formula (A), the N_{M} at positions 4, 6, and 10 in the antisense strand counting from the 5′ end are each a 2′-O-methyl modified nucleotide, and the N_{M} at position 12 in the antisense strand counting from the 5′ end is a 2′-fluoro modified nucleotide. In some embodiments in which the RNAi construct comprises a structure represented by Formula (A), each N_{M} in the sense strand is a 2′-O-methyl modified nucleotide. In other embodiments, each N_{M} in the sense strand is a 2′-fluoro modified nucleotide. In still other embodiments in which the RNAi construct comprises a structure represented by Formula (A), each N_{M} in both the sense and antisense strands is a 2′-O-methyl modified nucleotide.

In any of the above-described embodiments in which the RNAi construct comprises a structure represented by Formula (A), each N_{L} in both the sense and antisense strands can be a 2′-O-methyl modified nucleotide. In these embodiments and any of the embodiments described above, N_{T} in Formula (A) can be an inverted abasic nucleotide, an inverted deoxyribonucleotide, or a 2′-O-methyl modified nucleotide.

In certain embodiments of the invention, the RNAi construct comprises a sense strand and an antisense strand, wherein the antisense strand comprises a sequence that is complementary to a target gene sequence and the sense strand comprises a sequence that is sufficiently complementary to the sequence of the antisense strand to form a duplex region, wherein the RNAi construct comprises a structure represented by Formula (B):

(B)
5′-(N_{A})ₓ N_{L} N_{L} N_{L} N_{L} N_{L} N_{L} N_{F} N_{L} N_{F} N_{F} N_{F} N_{F} N_{L} N_{L}
N_{L} N_{L} N_{L} N_{L} N_{T} (n)_{y}-3′
3′-(N_{B})_{z} N_{L} N_{L} N_{L} N_{L} N_{L} N_{F} N_{L} N_{F} N_{L} N_{L} N_{L} N_{L} N_{F} N_{F}
N_{L} N_{F} N_{L} N_{F} N_{L}-5′

wherein:

the top strand listed in the 5′ to 3′ direction is the sense strand and the bottom strand listed in the 3′ to 5′ direction is the antisense strand;

each N_{F} represents a 2′-fluoro modified nucleotide;

each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

x is an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;

y is an integer from 0 to 4, provided that when y is 1, 2, 3, or 4, one or more n nucleotides are modified or unmodified overhang nucleotides that do not base pair with nucleotides in the antisense strand; and

z is an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

In some embodiments in which the RNAi construct comprises a structure represented by Formula (B), there is a nucleotide overhang at the 3′ end of the sense strand—i.e. y is 1, 2, 3, or 4. In one such embodiment, y is 2. In embodiments in which there is an overhang of 2 nucleotides at the 3′ end of the sense strand (i.e. y is 2), x is 0 and z is 2 or x is 1 and z is 2. In other embodiments in which the RNAi construct comprises a structure represented by Formula (B), the RNAi construct comprises a blunt end at the 3′ end of the sense strand and the 5′ end of the antisense strand (i.e. y is 0). In such embodiments where there is no nucleotide overhang at the 3′ end of the sense strand (i.e. y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In any of the above-described embodiments in which the RNAi construct comprises a structure represented by Formula (B), each N_{L} in both the sense and antisense strands can be a 2′-O-methyl modified nucleotide. In such embodiments and any of the embodiments described above, N_{T} in Formula (B) can be an inverted abasic nucleotide, an inverted deoxyribonucleotide, or a 2′-O-methyl modified nucleotide.

In some embodiments of the invention, the RNAi construct comprises a sense strand and an antisense strand, wherein the antisense strand comprises a sequence that is complementary to a target gene sequence and the sense strand comprises a sequence that is sufficiently complementary to the sequence of the antisense strand to form a duplex region, wherein the RNAi construct comprises a structure represented by Formula (C):

(C)
5′-(AB)ₓ N_{L} N_{L} N_{L} N_{L} N_{L} N_{L} N_{L} N_{L} N_{F} N_{L} N_{F} N_{F} N_{F} N_{F}
N_{L} N_{L} N_{M} N_{L} N_{M} N_{L} N_{T}-3′
3′-N_{L} N_{L} N_{L} N_{L} N_{L} N_{L} N_{L} N_{L} N_{L} N_{F} N_{L} N_{F} N_{L} N_{L} N_{L} N_{L}
N_{F} N_{L} N_{L} N_{M} N_{L} N_{F} N_{L}-5′

wherein:

the top strand listed in the 5′ to 3′ direction is the sense strand and the bottom strand listed in the 3′ to 5′ direction is the antisense strand;

each N_{F} represents a 2′-fluoro modified nucleotide;

each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and

x is 0 or 1 and Ab is an inverted abasic nucleotide.

In certain embodiments in which the RNAi construct comprises a structure represented by Formula (C), the N_{M} in the antisense strand is a 2′-fluoro modified nucleotide. In these and other embodiments, each N_{M} in the sense strand is a 2′-O-methyl modified nucleotide. In alternative embodiments, each N_{M} in the sense strand is a 2′-fluoro modified nucleotide. In some embodiments in which the RNAi construct comprises a structure represented by Formula (C), each N_{M} in both the sense and antisense strands is a 2′-O-methyl modified nucleotide.

In any of the above-described embodiments in which the RNAi construct comprises a structure represented by Formula (C), each N_{L} in both the sense and antisense strands can be a 2′-O-methyl modified nucleotide. In these embodiments and any of the embodiments described above, N_{T} in Formula (C) can be an inverted abasic nucleotide, an inverted deoxyribonucleotide, or a 2′-O-methyl modified nucleotide. For instance, in one embodiment, N_{T} is an inverted abasic nucleotide or inverted deoxyribonucleotide and x is 0. In another embodiment, N_{T} is a 2′-O-methyl modified nucleotide and x is 1. In yet another embodiment, N_{T} is an inverted abasic nucleotide or inverted deoxyribonucleotide and x is 1.

In certain embodiments, the RNAi construct of the invention comprises a sense strand and an antisense strand, wherein the antisense strand comprises a sequence that is complementary to a target gene sequence and the sense strand comprises a sequence that is sufficiently complementary to the sequence of the antisense strand to form a duplex region, wherein the RNAi construct comprises a structure represented by Formula (D):

(D)
5′-(N_{A})ₓ N_{L} N_{L} N_{L} N_{L} N_{M} N_{L} N_{F} N_{F} N_{F} N_{F} N_{L} N_{L} N_{L} N_{L}
N_{L} N_{L} N_{L} N_{L} N_{T}(n)_{y}-3′
3′-(N_{B})_{z} N_{L} N_{L} N_{L} N_{M} N_{L} N_{F} N_{L} N_{M} N_{L} N_{L} N_{M} N_{M} N_{M} N_{M}
N_{L} N_{M} N_{L} N_{F} N_{L}-5′

wherein:

the top strand listed in the 5′ to 3′ direction is the sense strand and the bottom strand listed in the 3′ to 5′ direction is the antisense strand;

each N_{F} represents a 2′-fluoro modified nucleotide;

each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a bicyclic nucleic acid (BNA), and a deoxyribonucleotide;

each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;

x is an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;

y is an integer from 0 to 4, provided that when y is 1, 2, 3, or 4, one or more n nucleotides are modified or unmodified overhang nucleotides that do not base pair with nucleotides in the antisense strand; and

z is an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

In some embodiments in which the RNAi construct comprises a structure represented by Formula (D), there is a nucleotide overhang at the 3′ end of the sense strand—i.e. y is 1, 2, 3, or 4. In one such embodiment, y is 2. In embodiments in which there is an overhang of 2 nucleotides at the 3′ end of the sense strand (i.e. y is 2), x is 0 and z is 2 or x is 1 and z is 2. In other embodiments in which the RNAi construct comprises a structure represented by Formula (D), the RNAi construct comprises a blunt end at the 3′ end of the sense strand and the 5′ end of the antisense strand (i.e. y is 0). In such embodiments where there is no nucleotide overhang at the 3′ end of the sense strand (i.e. y is 0): (i) x is 2 and z is 4, (ii) x is 3 and z is 4, (iii) x is 0 and z is 2, (iv) x is 1 and z is 2, or (v) x is 2 and z is 2. In any of the embodiments in which x is greater than 0, the N_{A} nucleotide that is the terminal nucleotide at the 5′ end of the sense strand can be an inverted nucleotide, such as an inverted abasic nucleotide or an inverted deoxyribonucleotide.

In certain embodiments in which the RNAi construct comprises a structure represented by Formula (D), the N_{M} at positions 4, 6, 8, 9, and 16 in the antisense strand counting from the 5′ end are each a 2′-fluoro modified nucleotide and the N_{M} at positions 7 and 12 in the antisense strand counting from the 5′ end are each a 2′-O-methyl modified nucleotide. In other embodiments, the N_{M} at positions 4 and 6 in the antisense strand counting from the 5′ end are each a 2′-fluoro modified nucleotide and the N_{M} at positions 7 to 9 in the antisense strand counting from the 5′ end are each a 2′-O-methyl modified nucleotide. In still other embodiments, the N_{M} at positions 4, 6, 8, 9, and 16 in the antisense strand counting from the 5′ end are each a 2′-O-methyl modified nucleotide and the N_{M} at positions 7 and 12 in the antisense strand counting from the 5′ end are each a 2′-fluoro modified nucleotide. In alternative embodiments in which the RNAi construct comprises a structure represented by Formula (D), the N_{M} at positions 4, 6, 8, 9, and 12 in the antisense strand counting from the 5′ end are each a 2′-O-methyl modified nucleotide and the N_{M} at positions 7 and 16 in the antisense strand counting from the 5′ end are each a 2′-fluoro modified nucleotide. In certain other embodiments in which the RNAi construct comprises a structure represented by Formula (D), the N_{M} at positions 7, 8, 9, and 12 in the antisense strand counting from the 5′ end are each a 2′-O-methyl modified nucleotide and the N_{M} at positions 4, 6, and 16 in the antisense strand counting from the 5′ end are each a 2′-fluoro modified nucleotide. In these and other embodiments in which the RNAi construct comprises a structure represented by Formula (D), the N_{M} in the sense strand is a 2′-fluoro modified nucleotide. In alternative embodiments, the N_{M} in the sense strand is a 2′-O-methyl modified nucleotide.

In any of the above-described embodiments in which the RNAi construct comprises a structure represented by Formula (D), each N_{L} in both the sense and antisense strands can be a 2′-O-methyl modified nucleotide. In these embodiments and any of the embodiments described above, N_{T} in Formula (D) can be an inverted abasic nucleotide, an inverted deoxyribonucleotide, or a 2′-O-methyl modified nucleotide.

The RNAi constructs of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the RNAi constructs of the invention are described in U.S. Pat. Nos. 6,693,187, 9,181,551, U.S. Patent Publication No. 2016/0122761, and Deleavey and Damha, Chemistry and Biology, Vol. 19: 937-954, 2012, all of which are hereby incorporated by reference in their entireties.

In certain embodiments, the RNAi constructs of the invention comprise one or more phosphorothioate internucleotide linkages. The phosphorothioate internucleotide linkages may be present in the sense strand, antisense strand, or both strands of the RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands.

In some embodiments, the RNAi construct comprises a single phosphorothioate internucleotide linkage between the terminal nucleotides at the 3′ end of the sense strand. In other embodiments, the RNAi construct comprises two consecutive phosphorothioate internucleotide linkages between the terminal nucleotides at the 3′ end of the sense strand. In one embodiment, the RNAi construct comprises a single phosphorothioate internucleotide linkage between the terminal nucleotides at the 3′ end of the sense strand and a single phosphorothioate internucleotide linkage between the terminal nucleotides at the 3′ end of the antisense strand. In another embodiment, the RNAi construct comprises two consecutive phosphorothioate internucleotide linkages between the terminal nucleotides at the 3′ end of the antisense strand (i.e. a phosphorothioate internucleotide linkage at the first and second internucleotide linkages at the 3′ end of the antisense strand). In another embodiment, the RNAi construct comprises two consecutive phosphorothioate internucleotide linkages between the terminal nucleotides at both the 3′ and 5′ ends of the antisense strand. In yet another embodiment, the RNAi construct comprises two consecutive phosphorothioate internucleotide linkages between the terminal nucleotides at both the 3′ and 5′ ends of the antisense strand and two consecutive phosphorothioate internucleotide linkages at the 5′ end of the sense strand. In still another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at the 3′ end of the sense strand. In another embodiment, the RNAi construct comprises two consecutive phosphorothioate internucleotide linkages between the terminal nucleotides at both the 3′ and 5′ ends of the antisense strand and two consecutive phosphorothioate internucleotide linkages between the terminal nucleotides at both the 3′ and 5′ ends of the sense strand (i.e. a phosphorothioate internucleotide linkage at the first and second internucleotide linkages at both the 5′ and 3′ ends of the antisense strand and a phosphorothioate internucleotide linkage at the first and second internucleotide linkages at both the 5′ and 3′ ends of the sense strand). In yet another embodiment, the RNAi construct comprises two consecutive phosphorothioate internucleotide linkages between the terminal nucleotides at both the 3′ and 5′ ends of the antisense strand and a single phosphorothioate internucleotide linkage between the terminal nucleotides at the 3′ end of the sense strand. In any of the embodiments in which one or both strands comprises one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In embodiments in which the RNAi construct comprises a nucleotide overhang, two or more of the unpaired nucleotides in the overhang can be connected by a phosphorothioate internucleotide linkage. In certain embodiments, all the unpaired nucleotides in a nucleotide overhang at the 3′ end of the antisense strand and/or the sense strand are connected by phosphorothioate internucleotide linkages. In other embodiments, all the unpaired nucleotides in a nucleotide overhang at the 5′ end of the antisense strand and/or the sense strand are connected by phosphorothioate internucleotide linkages. In still other embodiments, all the unpaired nucleotides in any nucleotide overhang are connected by phosphorothioate internucleotide linkages.

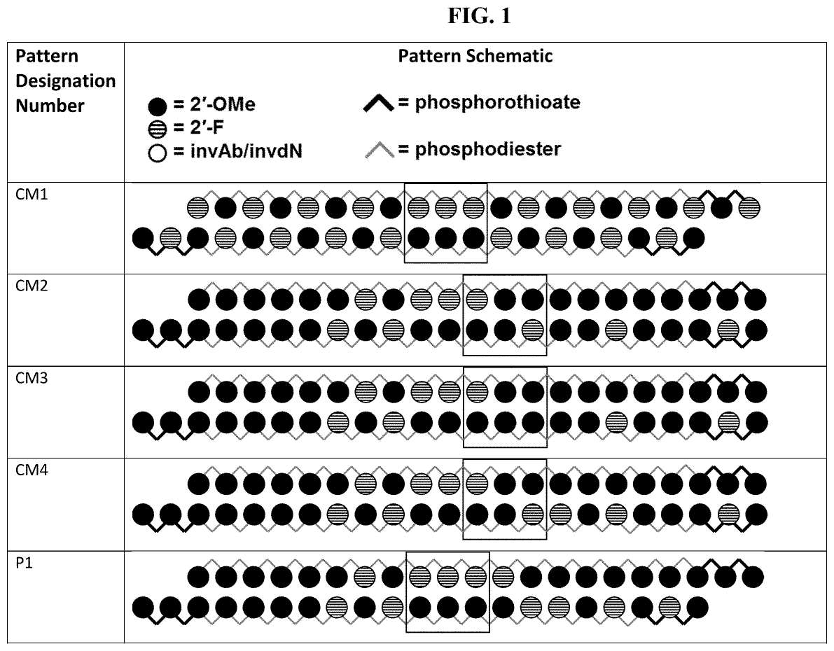

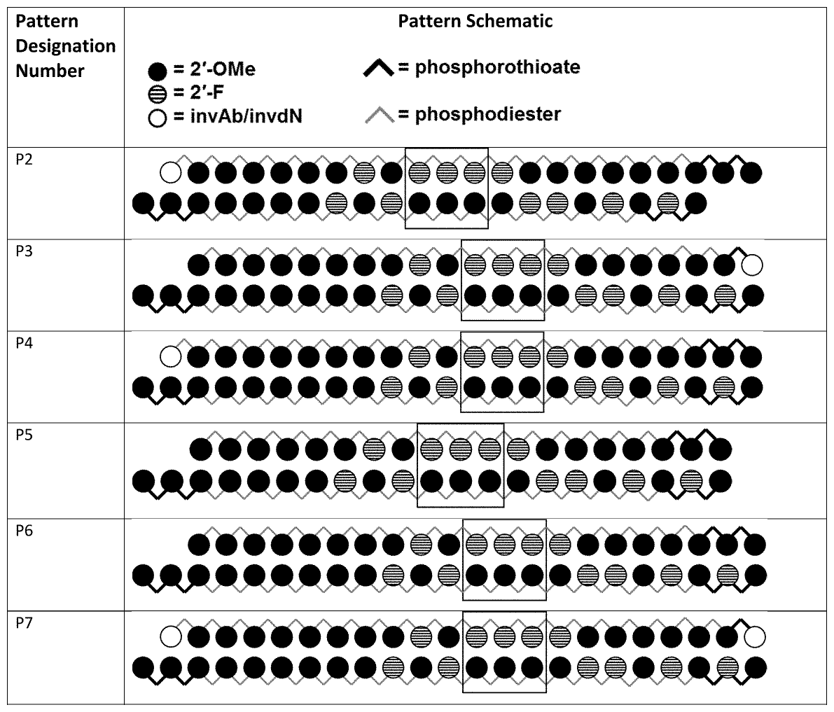

The RNAi constructs of the invention may have any one of the chemical modification patterns P1 through P30 depicted in FIG. 1. For instance, in some embodiments, the RNAi construct comprises a sense strand of 19-23 nucleotides in length and an antisense strand of 19-23 nucleotides in length, wherein the sequences of the antisense stand and the sense strand are sufficiently complementary to each other to form a duplex region of 19-21 base pairs, wherein: nucleotides at positions 2, 7, and 14 in the antisense strand (counting from the 5′ end) are 2′-fluoro modified nucleotides; nucleotides in the sense strand at positions paired with positions 8 to 11 and 13 in the antisense strand (counting from the 5′ end) are 2′-fluoro modified nucleotides; neither the sense strand nor the antisense strand each have more than 7 total 2′-fluoro modified nucleotides; and the RNAi construct has a nucleotide overhang at the 3′ ends of the sense strand and the antisense strand.

In one embodiment, the RNAi construct comprises:

(a) a sense strand having:
(i) a length of 21 nucleotides;
(ii) 2′-fluoro modified nucleotides at positions 7 and 9 to 12, and 2′-O-methyl modified nucleotides at positions 1 to 6, 8, and 13 to 21 (counting from the 5′ end); and
(iii) phosphorothioate internucleotide linkages between nucleotides at positions 19 and 20 and between nucleotides at positions 20 and 21 (counting from the 5′ end);
and

(b) an antisense strand having:
(i) a length of 21 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and the 3′ end of the antisense strand.

In another embodiment, the RNAi construct comprises:

(a) a sense strand having:
(i) a length of 22 nucleotides;
(ii) an inverted abasic nucleotide or inverted deoxyribonucleotide at position 1; 2′-fluoro modified nucleotides at positions 8 and 10 to 13; and 2′-O-methyl modified nucleotides at positions 2 to 7, 9, and 14 to 22 (counting from the 5′ end); and
(iii) phosphorothioate internucleotide linkages between nucleotides at positions 20 and 21 and between nucleotides at positions 21 and 22 (counting from the 5′ end);
and

(b) an antisense strand having:
a length of 21 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and a nucleotide overhang comprising 1 to 2 nucleotides at the 3′ end of the antisense strand.

In another embodiment, the RNAi construct comprises:

(a) a sense strand having:
(i) a length of 21 nucleotides;
(ii) 2′-fluoro modified nucleotides at positions 7 and 9 to 12, and 2′-O-methyl modified nucleotides at positions 1 to 6, 8, and 13 to 21 (counting from the 5′ end); and
(iii) phosphorothioate internucleotide linkages between nucleotides at positions 19 and 20 and between nucleotides at positions 20 and 21 (counting from the 5′ end);
and

(b) an antisense strand having:
(i) a length of 21 nucleotides;
(ii) 2′-fluoro modified nucleotides at positions 2, 7, 10, 12 and 14, and 2′-O-methyl modified nucleotides at positions 1, 3 to 6, 8, 9, 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and the 3′ end of the antisense strand.

In yet another embodiment, the RNAi construct comprises:

(a) a sense strand having:
(i) a length of 21 nucleotides;
(ii) 2′-fluoro modified nucleotides at positions 7 and 9 to 12, and 2′-O-methyl modified nucleotides at positions 1 to 6, 8, and 13 to 21 (counting from the 5′ end); and
(iii) phosphorothioate internucleotide linkages between nucleotides at positions 19 and 20 and between nucleotides at positions 20 and 21 (counting from the 5′ end);

and

(b) an antisense strand having:
(i) a length of 21 nucleotides;
(ii) 2′-fluoro modified nucleotides at positions 2, 4, 6, 7, 10, 12 and 14, and 2′-O-methyl modified nucleotides at positions 1, 3, 5, 8, 9, 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and the 3′ end of the antisense strand.

In another particular embodiment, the RNAi construct comprises:

(a) a sense strand having:
(i) a length of 21 nucleotides;
(ii) 2′-fluoro modified nucleotides at positions 7 and 9 to 12, and 2′-O-methyl modified nucleotides at positions 1 to 6, 8, and 13 to 20, and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end); wherein the RNAi construct has a nucleotide overhang comprising 2 nucleotides at the 3′ end of the sense strand and the 3′ end of the antisense strand.

In certain embodiments, the RNAi construct comprises a sense strand of 19-21 nucleotides in length and an antisense strand of 21-23 nucleotides in length, wherein the sequences of the antisense stand and the sense strand are sufficiently complementary to each other to form a duplex region of 19-21 base pairs, wherein: nucleotides at positions 2, 7, and 14 in the antisense strand (counting from the 5′ end) are 2′-fluoro modified nucleotides; nucleotides in the sense strand at positions paired with positions 8 to 11 and 13 in the antisense strand (counting from the 5′ end) are 2′-fluoro modified nucleotides; neither the sense strand nor the antisense strand each have more than 7 total 2′-fluoro modified nucleotides; and the RNAi construct has a nucleotide overhang at the 3′ end of the antisense strand and a blunt end at the 5′ end of the antisense strand/3′ end of the sense strand.

In one embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another embodiment, the RNAi construct comprises:

(a) a sense strand having:
(i) a length of 22 nucleotides;
(ii) an inverted abasic nucleotide or inverted deoxyribonucleotide at position 1; 2′-fluoro modified nucleotides at positions 10 and 12 to 15; and 2′-O-methyl modified nucleotides at positions 2 to 9, 11, and 16 to 22 (counting from the 5′ end); and
(iii) phosphorothioate internucleotide linkages between nucleotides at positions 20 and 21 and between nucleotides at positions 21 and 22 (counting from the 5′ end);
and

(b) an antisense strand having:
(i) a length of 23 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 1-2 nucleotides at the 3′ end of the antisense strand and a blunt end at the 5′ end of the antisense strand.

In yet another embodiment, the RNAi construct comprises:

(a) a sense strand having:
(i) a length of 21 nucleotides;
(ii) 2′-fluoro modified nucleotides at positions 9 and 11 to 14 and 2′-O-methyl modified nucleotides at positions 1 to 8, 10, and 15 to 21 (counting from the 5′ end); and
(iii) phosphorothioate internucleotide linkages between nucleotides at positions 19 and 20 and between nucleotides at positions 20 and 21 (counting from the 5′ end);
and

(b) an antisense strand having:
(i) a length of 23 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In still another embodiment, the RNAi construct comprises:

(a) a sense strand having:
(i) a length of 22 nucleotides;
(ii) an inverted abasic nucleotide or inverted deoxyribonucleotide at positions 1 and 22; 2′-fluoro modified nucleotides at positions 10 and 12 to 15; and 2′-O-methyl modified nucleotides at positions 2 to 9, 11, and 16 to 21 (counting from the 5′ end); and
(iii) a phosphorothioate internucleotide linkage between nucleotides at positions 21 and 22;
and

(b) an antisense strand having:
(i) a length of 23 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 1-2 nucleotides at the 3′ end of the antisense strand and a blunt end at the 5′ end of the antisense strand.

In one particular embodiment, the RNAi construct comprises:

(a) a sense strand having:
(i) a length of 19 nucleotides;
(ii) 2′-fluoro modified nucleotides at positions 7 and 9 to 12 and 2′-O-methyl modified nucleotides at positions 1 to 6, 8, and 13 to 19 (counting from the 5′ end); and
(iii) phosphorothioate internucleotide linkages between nucleotides at positions 17 and 18 and between nucleotides at positions 18 and 19 (counting from the 5′ end);
and

(b) an antisense strand having:
(i) a length of 21 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 19 nucleotides;
(ii) 2′-fluoro modified nucleotides at positions 7 and 9 to 12; 2′-O-methyl modified nucleotides at positions 1 to 6, 8, and 13 to 18; and an inverted abasic nucleotide or inverted deoxyribonucleotide at position 19 (counting from the 5′ end); and
(iii) phosphorothioate internucleotide linkages between nucleotides at positions 17 and 18 and between nucleotides at positions 18 and 19 (counting from the 5′ end);
and

(b) an antisense strand having:
(i) a length of 21 nucleotides;
(ii) 2′-fluoro modified nucleotides at positions 2, 4, 6, 7, 12 and 14, and 2′-O-methyl modified nucleotides at positions 1, 3, 5, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);

and

(b) an antisense strand having:
(i) a length of 23 nucleotides;
(ii) 2′-fluoro modified nucleotides at positions 2, 7, 12 and 14, and 2′-O-methyl modified nucleotides at positions 1, 3 to 6, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In yet another embodiment, the RNAi construct comprises:

(a) a sense strand having:
(i) a length of 22 nucleotides;
(ii) an inverted abasic nucleotide or inverted deoxyribonucleotide at position 1; 2′-fluoro modified nucleotides at positions 10 and 12 to 15; and 2′-O-methyl modified nucleotides at positions 2 to 9, 11, and 16 to 22 (counting from the 5′ end); and
(iii) phosphorothioate internucleotide linkages between nucleotides at positions 20 and 21 and between nucleotides at positions 21 and 22;
and

(b) an antisense strand having:
(i) a length of 23 nucleotides;
(ii) 2′-fluoro modified nucleotides at positions 2, 7, 12 and 14, and 2′-O-methyl modified nucleotides at positions 1, 3 to 6, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 1-2 nucleotides at the 3′ end of the antisense strand and a blunt end at the 5′ end of the antisense strand.

In still another embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 7, 12 and 14, and 2′-O-methyl modified nucleotides at positions 1, 3, 5, 6, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9, 11 to 14, 17, and 19; 2′-O-methyl modified nucleotides at positions 1 to 8, 10, 15, 16, 18 and 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 7, 12 and 14, and 2′-O-methyl modified nucleotides at positions 1, 3, 5, 6, 8 to 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 19 nucleotides;
(ii) 2′-fluoro modified nucleotides at positions 7 and 9 to 12; 2′-O-methyl modified nucleotides at positions 1 to 6, 8, and 13 to 18; and an inverted abasic nucleotide or inverted deoxyribonucleotide at position 19 (counting from the 5′ end); and
(iii) phosphorothioate internucleotide linkages between nucleotides at positions 18 and 19 and optionally between nucleotides at positions 17 and 18 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 6, 7, 10, 12 and 14, and 2′-O-methyl modified nucleotides at positions 1, 3, 5, 8, 9, 11, 13, and 15 to 23 (counting from the 5′ end); and 1
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 7, 10, 12 and 14, and 2′-O-methyl modified nucleotides at positions 1, 3 to 6, 8, 9, 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9, 11 to 14, 17, and 19; 2′-O-methyl modified nucleotides at positions 1 to 8, 10, 15, 16, 18, and 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 7, 10, 12 and 14, and 2′-O-methyl modified nucleotides at positions 1, 3 to 6, 8, 9, 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 9 and 11 to 14; 2′-O-methyl modified nucleotides at positions 1 to 8, 10, and 15 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 7, 10, 12 and 14, and 2′-O-methyl modified nucleotides at positions 1, 3, 5, 6, 8, 9, 11, 13, and 15 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In some embodiments of the invention, the RNAi construct comprises a sense strand of 19-23 nucleotides in length and an antisense strand of 19-23 nucleotides in length, wherein the sequences of the antisense stand and the sense strand are sufficiently complementary to each other to form a duplex region of 19-21 base pairs, wherein: nucleotides at positions 2, 14, and 16 in the antisense strand (counting from the 5′ end) are 2′-fluoro modified nucleotides; nucleotides in the sense strand at positions paired with positions 10 to 13 in the antisense strand (counting from the 5′ end) are 2′-fluoro modified nucleotides; and neither the sense strand nor the antisense strand each have more than 7 total 2′-fluoro modified nucleotides. In such embodiments, the RNAi construct has a nucleotide overhang at the 3′ end of the antisense strand and a blunt end at the 5′ end of the antisense strand/3′ end of the sense strand. In alternative embodiments, the RNAi construct has a nucleotide overhang at both of the 3′ ends of the sense strand and the antisense strand.

In one particular embodiment, the RNAi construct comprises:

(a) a sense strand having:
(i) a length of 21 nucleotides;
(ii) 2′-fluoro modified nucleotides at positions 7 and 9 to 12; 2′-O-methyl modified nucleotides at positions 1 to 6, 8, and 13 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 6, 8, 9, 14 and 16, and 2′-O-methyl modified nucleotides at positions 1, 3, 5, 7, 10 to 13, 15, and 17 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 7 and 9 to 12; 2′-O-methyl modified nucleotides at positions 1 to 6, 8, and 13 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 7, 14 and 16, and 2′-O-methyl modified nucleotides at positions 1, 3 to 6, 8 to 13, 15, and 17 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 21 nucleotides;
(ii) 2′-fluoro modified nucleotides at positions 7 and 9 to 12; 2′-O-methyl modified nucleotides at positions 1 to 6, 8, and 13 to 20; and an inverted abasic nucleotide or inverted deoxyribonucleotide at position 21 (counting from the 5′ end); and
(iii) a phosphorothioate internucleotide linkage between nucleotides at positions 20 and 21 (counting from the 5′ end);
and

(b) an antisense strand having:
(i) a length of 23 nucleotides;
(ii) 2′-fluoro modified nucleotides at positions 2, 4, 6, 14 and 16, and 2′-O-methyl modified nucleotides at positions 1, 3, 5, 7 to 13, 15, and 17 to 23 (counting from the 5′ end); and
(iii) phosphorothioate internucleotide linkages between nucleotides at positions 1 and 2, between nucleotides at positions 2 and 3, between nucleotides at positions 21 and 22, and between nucleotides at positions 22 and 23 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 19 nucleotides;
(ii) 2′-fluoro modified nucleotides at positions 5 and 7 to 10; 2′-O-methyl modified nucleotides at positions 1 to 4, 6, and 11 to 18; and an inverted abasic nucleotide or inverted deoxyribonucleotide at position 19 (counting from the 5′ end); and
(iii) a phosphorothioate internucleotide linkage between nucleotides at positions 18 and 19 (counting from the 5′ end);
and

(b) an antisense strand having:
(i) a length of 21 nucleotides;
(ii) 2′-fluoro modified nucleotides at positions 2, 4, 6, 8, 9, 14 and 16, and 2′-O-methyl modified nucleotides at positions 1, 3, 5, 7, 10 to 13, 15, and 17 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the antisense strand and a blunt end at the 5′ end of the antisense strand.

In another particular embodiment, the RNAi construct comprises:

(a) a sense strand having:
(i) a length of 20 nucleotides;
(ii) an inverted abasic nucleotide or inverted deoxyribonucleotide at position 1; 2′-fluoro modified nucleotides at positions 8 to 11; and 2′-O-methyl modified nucleotides at positions 2 to 7 and 12 to 20 (counting from the 5′ end); and
(iii) phosphorothioate internucleotide linkages between nucleotides at positions 18 and 19 and between nucleotides at positions 19 and 20 (counting from the 5′ end);
and

(b) an antisense strand having:
(i) a length of 21 nucleotides;
(ii) 2′-fluoro modified nucleotides at positions 2, 7, 14 and 16, and 2′-O-methyl modified nucleotides at positions 1, 3 to 6, 8 to 13, 15, and 17 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 1-2 nucleotides at the 3′ end of the antisense strand and a blunt end at the 5′ end of the antisense strand.

In another embodiment, the RNAi construct comprises:

(a) a sense strand having:
(i) a length of 22 nucleotides;
(ii) an inverted abasic nucleotide or inverted deoxyribonucleotide at position 1; 2′-fluoro modified nucleotides at positions 8 to 11; and 2′-O-methyl modified nucleotides at positions 2 to 7, and 12 to 22 (counting from the 5′ end); and
(iii) phosphorothioate internucleotide linkages between nucleotides at positions 20 and 21 and between nucleotides at positions 21 and 22 (counting from the 5′ end);
and

(b) an antisense strand having:
(i) a length of 21 nucleotides;
(ii) 2′-fluoro modified nucleotides at positions 2, 7, 14 and 16, and 2′-O-methyl modified nucleotides at positions 1, 3 to 6, 8 to 13, 15, and 17 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and a nucleotide overhang comprising 1 to 2 nucleotides at the 3′ end of the antisense strand.

In certain embodiments of the invention, the RNAi construct comprises a sense strand of 19-23 nucleotides in length and an antisense strand of 19-23 nucleotides in length, wherein the sequences of the antisense stand and the sense strand are sufficiently complementary to each other to form a duplex region of 19-21 base pairs, wherein: nucleotides at positions 2, 7, 12, and 14 in the antisense strand (counting from the 5′ end) are 2′-fluoro modified nucleotides; nucleotides in the sense strand at positions paired with positions 10 to 13 in the antisense strand (counting from the 5′ end) are 2′-fluoro modified nucleotides; neither the sense strand nor the antisense strand each have more than 7 total 2′-fluoro modified nucleotides; and the RNAi construct has a nucleotide overhang at the 3′ ends of the sense strand and the antisense strand.

For instance, in one embodiment, the RNAi construct comprises:

(a) a sense strand having:
(i) a length of 21 nucleotides;
(ii) 2′-fluoro modified nucleotides at positions 7 to 10; and 2′-O-methyl modified nucleotides at positions 1 to 6, and 11 to 21 (counting from the 5′ end); and
(iii) phosphorothioate internucleotide linkages between nucleotides at positions 19 and 20 and between nucleotides at positions 20 and 21 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and a nucleotide overhang comprising 2 nucleotides at the 3′ end of the antisense strand.

In another embodiment, the RNAi construct comprises:

(a) a sense strand having:
(i) a length of 22 nucleotides;
(ii) an inverted abasic nucleotide or inverted deoxyribonucleotide at position 1; 2′-fluoro modified nucleotides at positions 8 to 11; and 2′-O-methyl modified nucleotides at positions 2 to 7, and 12 to 22 (counting from the 5′ end); and
(iii) phosphorothioate internucleotide linkages between nucleotides at positions 20 and 21 and between nucleotides at positions 21 and 22 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has a nucleotide overhang comprising 2 nucleotides at the 3′ end of the sense strand and a nucleotide overhang comprising 1 to 2 nucleotides at the 3′ end of the antisense strand.

In certain embodiments of the invention, the RNAi construct comprises a sense strand of 19-21 nucleotides in length and an antisense strand of 19-21 nucleotides in length, wherein the sequences of the antisense stand and the sense strand are sufficiently complementary to each other to form a duplex region of 19-21 base pairs, wherein: nucleotides at positions 2, 7, 12, and 14 in the antisense strand (counting from the 5′ end) are 2′-fluoro modified nucleotides; nucleotides in the sense strand at positions paired with positions 10, 11, and 13 in the antisense strand (counting from the 5′ end) are 2′-fluoro modified nucleotides; and neither the sense strand nor the antisense strand each have more than 7 total 2′-fluoro modified nucleotides. In one such embodiment, the RNAi construct comprises:

(a) a sense strand having:
(i) a length of 21 nucleotides;
(ii) 2′-fluoro modified nucleotides at positions 9, and 11 to 14; and 2′-O-methyl modified nucleotides at positions 1 to 8, 10, and 15 to 20, and an inverted abasic nucleotide or inverted deoxyribonucleotide at position 21; (counting from the 5′ end); and
(iii) a phosphorothioate internucleotide linkage between nucleotides at positions 20 and 21 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has two blunt ends.

In another such embodiment, the RNAi construct comprises:

(a) a sense strand having:
(i) a length of 21 nucleotides;
(ii) 2′-fluoro modified nucleotides at positions 9 to 12; and 2′-O-methyl modified nucleotides at positions 1 to 8 and 13 to 20, and an inverted abasic nucleotide or inverted deoxyribonucleotide at position 21; (counting from the 5′ end); and
(iii) a phosphorothioate internucleotide linkage between nucleotides at positions 20 and 21 (counting from the 5′ end);
and

(b) an antisense strand having:
(i) a length of 21 nucleotides;
(ii) 2′-fluoro modified nucleotides at positions 2, 7, 12 and 14, and 2′-O-methyl modified nucleotides at positions 1, 3 to 6, 8 to 11, 13, and 15 to 21 (counting from the 5′ end); and
(iii) phosphorothioate internucleotide linkages between nucleotides at positions 1 and 2, between nucleotides at positions 2 and 3, between nucleotides at positions 19 and 20, and between nucleotides at positions 20 and 21 (counting from the 5′ end);

wherein the RNAi construct has two blunt ends.

In some embodiments of the invention, the 5′ end of the sense strand, antisense strand, or both the antisense and sense strands of the RNAi constructs comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups are replaced with H, O, S, N(R) or alkyl where R is H, an amino protecting group or unsubstituted or substituted alkyl. Exemplary phosphate moieties include, but are not limited to, 5′-monophosphate; 5′-diphosphate; 5′-triphosphate; 5′-guanosine cap (7-methylated or non-methylated); 5′-adenosine cap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (e.g., alkyl=methyl, ethyl, isopropyl, propyl, etc.); and 5′-alkyletherphosphonates (e.g., alkylether=methoxymethyl, ethoxymethyl, etc.).

The modified nucleotides that can be incorporated into the RNAi constructs of the invention may have more than one chemical modification described herein. For instance, the modified nucleotide may have a modification to the ribose sugar as well as a modification to the nucleobase. By way of example, a modified nucleotide may comprise a 2′ sugar modification (e.g. 2′-fluoro or 2′-O-methyl) and comprise a modified base (e.g. 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the invention comprise a combination of 2′ modified nucleotides or BNAs and phosphorothioate internucleotide linkages. In certain embodiments, both the sense and antisense strands of the RNAi constructs of the invention comprise a combination of 2′-fluoro modified nucleotides, 2′-O-methyl modified nucleotides, and phosphorothioate internucleotide linkages.

In certain embodiments, the nucleotide at position 1 of the antisense strand counting from the 5′ end in the RNAi constructs may comprise A, dA, dU, U, or dT. In some embodiments, at least one of the first three base pairs within the duplex region from the 5′ end of the antisense strand is an AU base pair. In one particular embodiment, the first base pair within the duplex region from the 5′ end of the antisense strand is an AU base pair.

The RNAi constructs of the invention can readily be made using techniques known in the art, for example, using conventional nucleic acid solid phase synthesis. The polynucleotides of the RNAi constructs can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, Calif. ), MerMade synthesizers from BioAutomation (Irving, Tex.), and OligoPilot synthesizers from GE Healthcare Life Sciences (Pittsburgh, Pa.). An exemplary method for synthesizing the RNAi constructs of the invention is described in Example 1.

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include, e.g., tetrazole, S-ethyl-tetrazole, benzylthiotetrazole, p-nitrophenyltetrazole.

As can be appreciated by the skilled artisan, further methods of synthesizing the RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the RNAi constructs described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi agents is also available from several commercial vendors, including Dharmacon, Inc. (Lafayette, Colo.), AxoLabs GmbH (Kulmbach, Germany), and Ambion, Inc. (Foster City, Calif.).

The RNAi constructs of the invention may comprise a ligand. As used herein, a “ligand” refers to any compound or molecule that is capable of interacting with another compound or molecule, directly or indirectly. The interaction of a ligand with another compound or molecule may elicit a biological response (e.g. initiate a signal transduction cascade, induce receptor-mediated endocytosis) or may just be a physical association. The ligand can modify one or more properties of the double-stranded RNA molecule to which is attached, such as the pharmacodynamic, pharmacokinetic, binding, absorption, cellular distribution, cellular uptake, charge and/or clearance properties of the RNA molecule.

The ligand may comprise a serum protein (e.g., human serum albumin, low-density lipoprotein, globulin), a cholesterol moiety, a vitamin (biotin, vitamin E, vitamin B₁₂), a folate moiety, a steroid, a bile acid (e.g. cholic acid), a fatty acid (e.g., palmitic acid, myristic acid), a carbohydrate (e.g., a dextran, pullulan, chitin, chitosan, inulin, cyclodextrin or hyaluronic acid), a glycoside, a phospholipid, or antibody or binding fragment thereof (e.g. antibody or binding fragment that targets the RNAi construct to a specific cell type, such as liver). Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, O3-(oleoyl)lithocholic acid, O3-(oleoyl)cholenic acid, dimethoxytrityl, or phenoxazine), peptides (e.g., antennapedia peptide, Tat peptide, RGD peptides), alkylating agents, polymers, such as polyethylene glycol (PEG)(e.g., PEG-40K), polyamino acids, and polyamines (e.g. spermine, spermidine).

In certain embodiments, the ligands have endosomolytic properties. The endosomolytic ligands promote the lysis of the endosome and/or transport of the RNAi construct of the invention, or its components, from the endosome to the cytoplasm of the cell. The endosomolytic ligand may be a polycationic peptide or peptidomimetic, which shows pH-dependent membrane activity and fusogenicity. In one embodiment, the endosomolytic ligand assumes its active conformation at endosomal pH. The “active” conformation is that conformation in which the endosomolytic ligand promotes lysis of the endosome and/or transport of the RNAi construct of the invention, or its components, from the endosome to the cytoplasm of the cell. Exemplary endosomolytic ligands include the GALA peptide (Subbarao et al., Biochemistry, Vol. 26: 2964-2972, 1987), the EALA peptide (Vogel et al., J. Am. Chem. Soc., Vol. 118: 1581-1586, 1996), and their derivatives (Turk et al., Biochem. Biophys. Acta, Vol. 1559: 56-68, 2002). In one embodiment, the endosomolytic component may contain a chemical group (e.g., an amino acid) which will undergo a change in charge or protonation in response to a change in pH. The endosomolytic component may be linear or branched.

In some embodiments, the ligand comprises a lipid or other hydrophobic molecule. In one embodiment, the ligand comprises a cholesterol moiety or other steroid. Cholesterol-conjugated oligonucleotides have been reported to be more active than their unconjugated counterparts (Manoharan, Antisense Nucleic Acid Drug Development, Vol. 12: 103-228, 2002). Ligands comprising cholesterol moieties and other lipids for conjugation to nucleic acid molecules have also been described in U.S. Pat. Nos. 7,851,615; 7,745,608; and 7,833,992, all of which are hereby incorporated by reference in their entireties. In another embodiment, the ligand comprises a folate moiety. Polynucleotides conjugated to folate moieties can be taken up by cells via a receptor-mediated endocytosis pathway. Such folate-polynucleotide conjugates are described in U.S. Pat. No. 8,188,247, which is hereby incorporated by reference in its entirety.

The ligand can target the RNAi construct to a specific tissue or cell type to selectively inhibit the expression of the target gene in that specific tissue or cell type. In one embodiment, the ligand targets delivery of the RNAi construct specifically to liver cells (e.g. hepatocytes) using various approaches as described in more detail below. In certain embodiments, the RNAi constructs are targeted to liver cells with a ligand that binds to the surface-expressed asialoglycoprotein receptor (ASGR) or component thereof (e.g. ASGR1, ASGR2).

In some embodiments, RNAi constructs can be specifically targeted to the liver by employing ligands that bind to or interact with proteins expressed on the surface of liver cells. For example, in certain embodiments, the ligands may comprise antigen binding proteins (e.g. antibodies or binding fragments thereof (e.g. Fab, scFv)) that specifically bind to a receptor expressed on hepatocytes, such as the asialoglycoprotein receptor and the LDL receptor. In one particular embodiment, the ligand comprises an antibody or binding fragment thereof that specifically binds to ASGR1 and/or ASGR2. In another embodiment, the ligand comprises a Fab fragment of an antibody that specifically binds to ASGR1 and/or ASGR2. A “Fab fragment” is comprised of one immunoglobulin light chain
(i.e. light chain variable region (VL) and constant region (CL)) and the CH1 region and variable region (VH) of one immunoglobulin heavy chain. In another embodiment, the ligand comprises a single-chain variable antibody fragment (scFv fragment) of an antibody that specifically binds to ASGR1 and/or ASGR2. An “scFv fragment” comprises the VH and VL regions of an antibody, wherein these regions are present in a single polypeptide chain, and optionally comprising a peptide linker between the VH and VL regions that enables the Fv to form the desired structure for antigen binding. Exemplary antibodies and binding fragments thereof that specifically bind to ASGR1 that can be used as ligands for targeting the RNAi constructs of the invention to the liver are described in WIPO Publication No. WO 2017/058944, which is hereby incorporated by reference in its entirety. Other antibodies or binding fragments thereof that specifically bind to ASGR1, LDL receptor, or other liver surface-expressed proteins suitable for use as ligands in the RNAi constructs of the invention are commercially available.

In certain embodiments, the ligand comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the ligand is a monosaccharide selected from a pentose, hexose, or heptose and di- and tri-saccharides including such monosaccharide units. In other embodiments, the carbohydrate incorporated into the ligand is an amino sugar, such as galactosamine, glucosamine, N-acetylgalactosamine, and N-acetylglucosamine.

In some embodiments, the ligand comprises a hexose or hexosamine. The hexose may be selected from glucose, galactose, mannose, fucose, or fructose. The hexosamine may be selected from fructosamine, galactosamine, glucosamine, or mannosamine. In certain embodiments, the ligand comprises glucose, galactose, galactosamine, or glucosamine. In one embodiment, the ligand comprises glucose, glucosamine, or N-acetylglucosamine. In another embodiment, the ligand comprises galactose, galactosamine, or N-acetyl-galactosamine. In particular embodiments, the ligand comprises N-acetyl-galactosamine. Ligands comprising glucose, galactose, and N-acetyl-galactosamine (GalNAc) are particularly effective in targeting compounds to liver cells because such ligands bind to the ASGR expressed on the surface of hepatocytes. See, e.g., D′Souza and Devaraj an, J. Control Release, Vol. 203: 126-139, 2015. Examples of GalNAc- or galactose-containing ligands that can be incorporated into the RNAi constructs of the invention are described in U.S. Pat. Nos. 7,491,805; 8,106,022; and 8,877,917; U.S. Patent Publication No. 20030130186; and WIPO Publication No. WO 2013166155, all of which are hereby incorporated by reference in their entireties.

In certain embodiments, the ligand comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the ligand comprises a multivalent galactose moiety. In other embodiments, the ligand comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent. Exemplary trivalent and tetravalent GalNAc-containing ligands for incorporation into the RNAi constructs of the invention are described in detail below.

The ligand can be attached or conjugated to the RNA molecule of the RNAi construct directly or indirectly. For instance, in some embodiments, the ligand is covalently attached directly to the sense or antisense strand of the RNAi construct. In other embodiments, the ligand is covalently attached via a linker to the sense or antisense strand of the RNAi construct. The ligand can be attached to nucleobases, sugar moieties, or internucleotide linkages of polynucleotides (e.g. sense strand or antisense strand) of the RNAi constructs of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a ligand. Conjugation or attachment to pyrimidine nucleobases or derivatives thereof can also occur at any position. In some embodiments, the 2-, 5-, and 6-positions of a pyrimidine nucleobase can be attached to a ligand. Conjugation or attachment to sugar moieties of nucleotides can occur at any carbon atom. Exemplary carbon atoms of a sugar moiety that can be attached to a ligand include the 2′, 3′, and 5′ carbon atoms. The 1′ position can also be attached to a ligand, such as in an abasic nucleotide. Internucleotide linkages can also support ligand attachments. For phosphorus-containing linkages (e.g., phosphodiester, phosphorothioate, phosphorodithiotate, phosphoroamidate, and the like), the ligand can be attached directly to the phosphorus atom or to an O, N, or S atom bound to the phosphorus atom. For amine- or amide-containing internucleoside linkages (e.g., PNA), the ligand can be attached to the nitrogen atom of the amine or amide or to an adjacent carbon atom.

In certain embodiments, the ligand may be attached to the 3′ or 5′ end of either the sense or antisense strand. In certain embodiments, the ligand is covalently attached to the 5′ end of the sense strand. In such embodiments, the ligand is attached to the 5′-terminal nucleotide of the sense strand. In these and other embodiments, the ligand is attached at the 5′-position of the 5′-terminal nucleotide of the sense strand. In embodiments in which an inverted abasic nucleotide or inverted deoxyribonucleotide is the 5′-terminal nucleotide of the sense strand and linked to the adjacent nucleotide via a 5′-5′ internucleotide linkage, the ligand can be attached at the 3′-position of the inverted abasic nucleotide or inverted deoxyribonucleotide. In other embodiments, the ligand is covalently attached to the 3′ end of the sense strand. For example, in some embodiments, the ligand is attached to the 3′-terminal nucleotide of the sense strand. In certain such embodiments, the ligand is attached at the 3′-position of the 3′-terminal nucleotide of the sense strand. In embodiments in which an inverted abasic nucleotide or inverted deoxyribonucleotide is the 3′-terminal nucleotide of the sense strand and linked to the adjacent nucleotide via a 3′-3′ internucleotide linkage, the ligand can be attached at the 5′-position of the inverted abasic nucleotide or inverted deoxyribonucleotide. In alternative embodiments, the ligand is attached near the 3′ end of the sense strand, but before one or more terminal nucleotides (i.e. before 1, 2, 3, or 4 terminal nucleotides). In some embodiments, the ligand is attached at the 2′-position of the sugar of the 3′-terminal nucleotide of the sense strand. In other embodiments, the ligand is attached at the 2′-position of the sugar of the 5′-terminal nucleotide of the sense strand.

In certain embodiments, the ligand is attached to the sense or antisense strand via a linker. A “linker” is an atom or group of atoms that covalently joins a ligand to a polynucleotide component of the RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g. sense or antisense strand of the RNAi construct) and the other is selected to bind essentially any selected group, such as a ligand as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a ligand to the sense or antisense strand in the RNAi constructs of the invention include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl.

In certain embodiments, the linkers are cleavable. A cleavable linker is one which is sufficiently stable outside the cell, but which upon entry into a target cell is cleaved to release the two parts the linker is holding together. In some embodiments, the cleavable linker is cleaved at least 10 times, 20 times, 30 times, 40 times, 50 times, 60 times, 70 times, 80 times, 90 times, or more, or at least 100 times faster in the target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linkers are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linker by reduction; esterases; endosomes or agents that can create an acidic environment, e.g., those that result in a pH of five or lower; enzymes that can hydrolyze or degrade an acid cleavable linker by acting as a general acid, peptidases (which can be substrate specific), and phosphatases.

A cleavable linker may comprise a moiety that is susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable group that is cleaved at a preferred pH, thereby releasing the RNA molecule from the ligand inside the cell, or into the desired compartment of the cell.

A linker can include a cleavable group that is cleavable by a particular enzyme. The type of cleavable group incorporated into a linker can depend on the cell to be targeted. For example, liver-targeting ligands can be linked to RNA molecules through a linker that includes an ester group. Liver cells are rich in esterases, and therefore the linker will be cleaved more efficiently in liver cells than in cell types that are not esterase-rich. Other types of cells rich in esterases include cells of the lung, renal cortex, and testis. Linkers that contain peptide bonds can be used when targeting cells rich in peptidases, such as liver cells and synoviocytes.

In general, the suitability of a candidate cleavable linker can be evaluated by testing the ability of a degradative agent (or condition) to cleave the candidate linker. It will also be desirable to also test the candidate cleavable linker for the ability to resist cleavage in the blood or when in contact with non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In some embodiments, useful candidate linkers are cleaved at least 2, 4, 10, 20, 50, 70, or 100 times faster in the cell (or under in vitro conditions selected to mimic intracellular conditions) as compared to blood or serum (or under in vitro conditions selected to mimic extracellular conditions).

In other embodiments, redox cleavable linkers are utilized. Redox cleavable linkers are cleaved upon reduction or oxidation. An example of a reductively cleavable group is a disulfide linking group (—S—S—). To determine if a candidate cleavable linker is a suitable “reductively cleavable linker,” or for example is suitable for use with a particular RNAi construct and particular ligand, one can use one or more methods described herein. For example, a candidate linker can be evaluated by incubation with dithiothreitol (DTT), or other reducing agent known in the art, which mimics the rate of cleavage that would be observed in a cell, e.g., a target cell. The candidate linkers can also be evaluated under conditions which are selected to mimic blood or serum conditions. In a specific embodiment, candidate linkers are cleaved by at most 10% in the blood. In other embodiments, useful candidate linkers are degraded at least 2, 4, 10, 20, 50, 70, or 100 times faster in the cell (or under in vitro conditions selected to mimic intracellular conditions) as compared to blood (or under in vitro conditions selected to mimic extracellular conditions).

In yet other embodiments, phosphate-based cleavable linkers, which are cleaved by agents that degrade or hydrolyze the phosphate group, are employed to covalently attach a ligand to the sense or antisense strand of the RNAi construct. An example of an agent that hydrolyzes phosphate groups in cells are enzymes, such as phosphatases in cells. Examples of phosphate-based cleavable groups are —O—P(O)(ORk)-O—, —O—P(S)(ORk)-O—, —O—P(S)(SRk)-O—, —S—P(O) (ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, and —O—P(S)(Rk)-S—, where Rk can be hydrogen or alkyl. Specific embodiments include —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—. Another specific embodiment is —O—P(O)(OH)—O—. These candidate linkers can be evaluated using methods analogous to those described above.

In other embodiments, the linkers may comprise acid cleavable groups, which are groups that are cleaved under acidic conditions. In some embodiments, acid cleavable groups are cleaved in an acidic environment with a pH of about 6.5 or lower (e.g., about 6.0, 5.5, 5.0, or lower), or by agents, such as enzymes that can act as a general acid. In a cell, specific low pH organelles, such as endosomes and lysosomes, can provide a cleaving environment for acid cleavable groups. Examples of acid cleavable linking groups include, but are not limited to, hydrazones, esters, and esters of amino acids. Acid cleavable groups can have the general formula —C═NN—, C(O)O, or —OC(O). A specific embodiment is when the carbon attached to the oxygen of the ester (the alkoxy group) is an aryl group, substituted alkyl group, or tertiary alkyl group such as dimethyl, pentyl or t-butyl. These candidates can be evaluated using methods analogous to those described above.

In other embodiments, the linkers may comprise ester-based cleavable groups, which are cleaved by enzymes, such as esterases and amidases in cells. Examples of ester-based cleavable groups include, but are not limited to, esters of alkylene, alkenylene and alkynylene groups. Ester cleavable groups have the general formula —C(O)O—, or —OC(O)—. These candidate linkers can be evaluated using methods analogous to those described above.

In further embodiments, the linkers may comprise peptide-based cleavable groups, which are cleaved by enzymes, such as peptidases and proteases in cells. Peptide-based cleavable groups are peptide bonds formed between amino acids to yield oligopeptides (e.g., dipeptides, tripeptides etc.) and polypeptides. Peptide-based cleavable groups include the amide group (—C(O)NH—). The amide group can be formed between any alkylene, alkenylene or alkynylene. A peptide bond is a special type of amide bond formed between amino acids to yield peptides and proteins. The peptide-based cleavage group is generally limited to the peptide bond (i.e., the amide bond) formed between amino acids yielding peptides and proteins. Peptide-based cleavable linking groups have the general formula —NHCHR^{A}C(O)NHCHR^{B}C(O)—, where R^{A} and R^{B} are the side chains of the two adjacent amino acids. These candidates can be evaluated using methods analogous to those described above.

Other types of linkers suitable for attaching ligands to the sense or antisense strands in the RNAi constructs of the invention are known in the art and can include the linkers described in U.S. Pat. Nos. 7,723,509; 8,017,762; 8,828,956; 8,877,917; and 9,181,551, all of which are hereby incorporated by reference in their entireties.

In certain embodiments, the ligand covalently attached to the sense or antisense strand of the RNAi constructs of the invention comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In certain embodiments, the RNAi constructs of the invention comprise a ligand having the following structure:

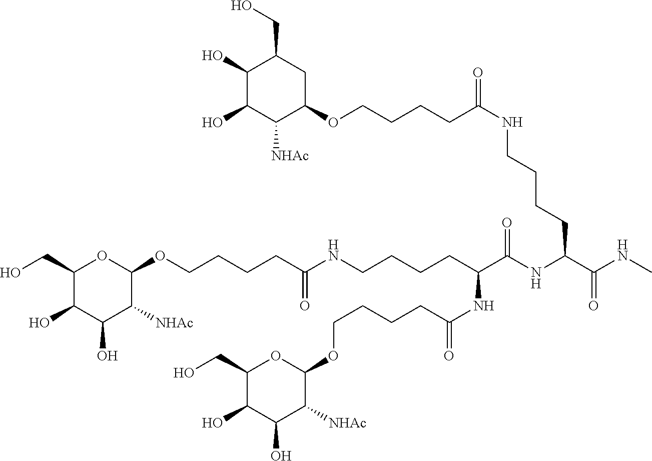 [SMILES: CNC(=O)[C@H](CCCCNC(=O)CCCCO[C@@H]1C[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In preferred embodiments, the ligand having this structure is covalently attached to the 5′ end of the sense strand via a linker, such as the linkers described herein. In one embodiment, the linker is an aminohexyl linker.

Exemplary trivalent and tetravalent GalNAc moieties and linkers that can be attached to the double-stranded RNA molecules in the RNAi constructs of the invention are provided in the structural formulas I-IX below. “Ac” in the formulas listed herein represents an acetyl group.

In one embodiment, the RNAi construct comprises a ligand and linker having the following structure of Formula I, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

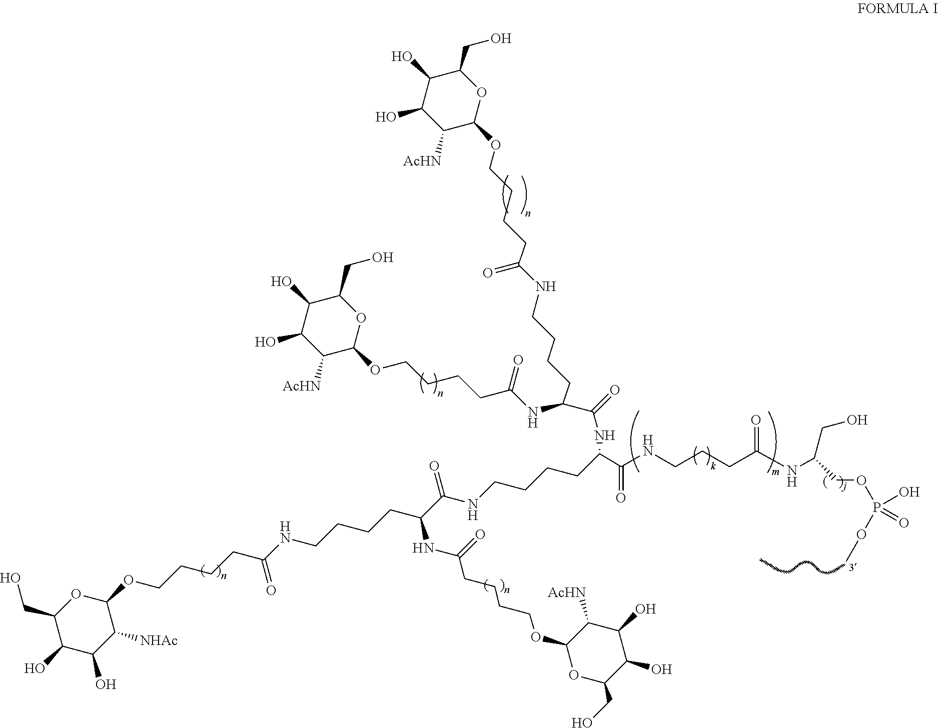

In another embodiment, the RNAi construct comprises a ligand and linker having the following structure of Formula II, wherein each n is independently 1 to 3, k is 1 to 3, m is 1 or 2, j is 1 or 2, and the ligand is attached to the 3′ end of the sense strand of the double-stranded RNA molecule (represented by the solid wavy line):

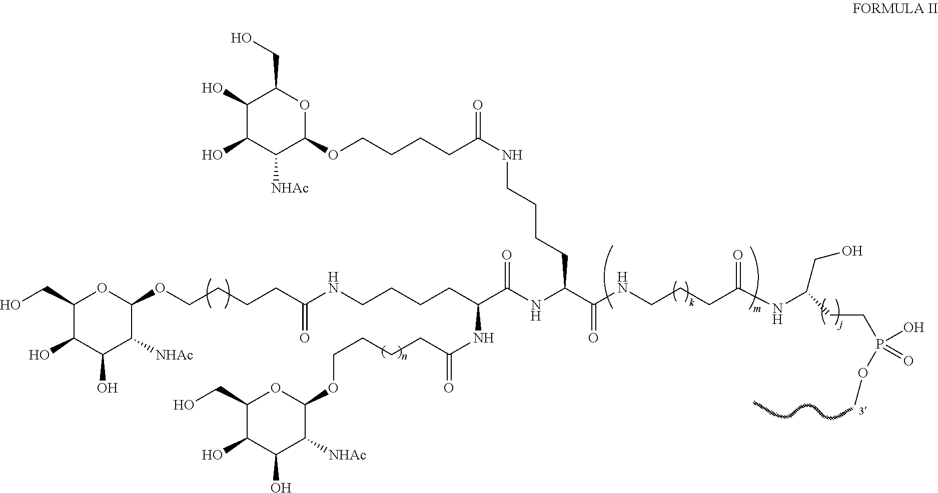

In yet another embodiment, the RNAi construct comprises a ligand and linker having the following structure of Formula III, wherein the ligand is attached to the 3′ end of the sense strand of the double-stranded RNA molecule (represented by the solid wavy line):

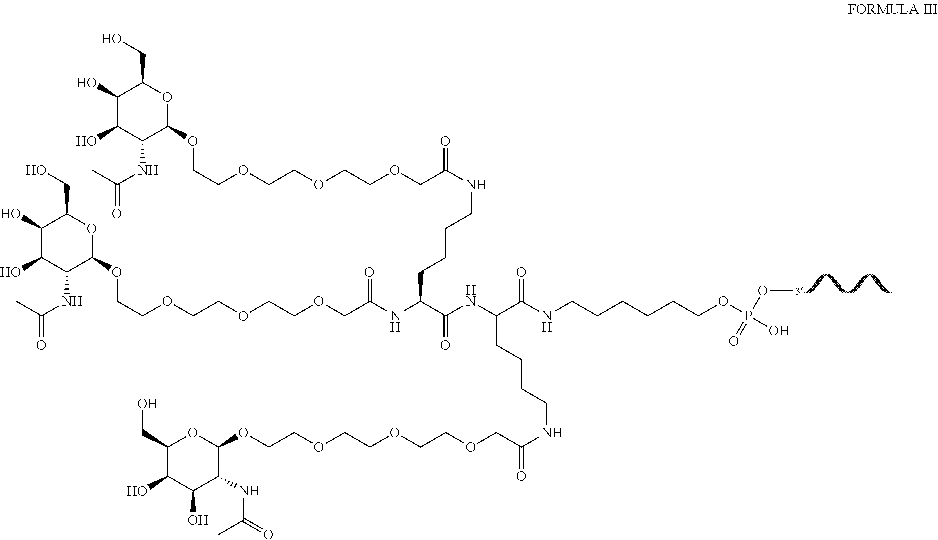 [SMILES: COP(=O)(O)OCCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

In still another embodiment, the RNAi construct comprises a ligand and linker having the following structure of Formula IV, wherein the ligand is attached to the 3′ end of the sense strand of the double-stranded RNA molecule (represented by the solid wavy line):

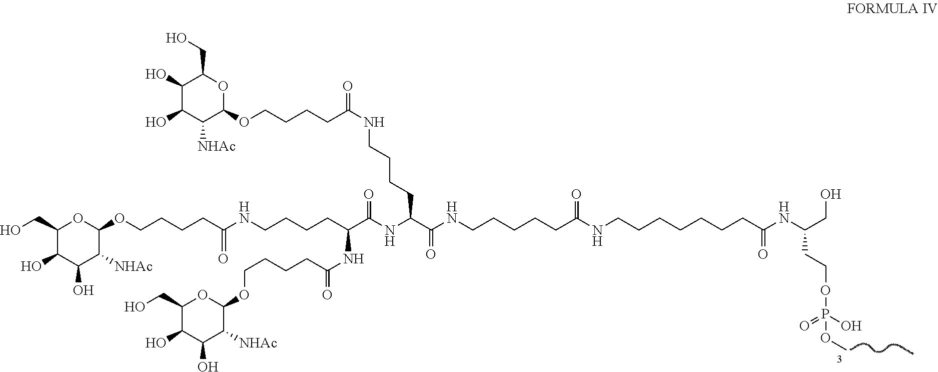 [SMILES: COP(=O)(O)OCC[C@@H](CO)NC(=O)CCCCCCCNC(=O)CCCCCNC(=O)[C@H](CCCCNC(=O)CCCCO[C@@H]1O[C@H](CO)[C@H](O)[C@H](O)[C@H]1NC(C)=O)NC(=O)[C@H](CCCCNC(=O)CCCCO[C@@H]1O[C@H](CO)[C@H](O)[C@H](O)[C@H]1NC(C)=O)NC(=O)CCCCO[C@@H]1O[C@H](CO)[C@H](O)[C@H](O)[C@H]1NC(C)=O]

In certain embodiments, the RNAi construct comprises a ligand and linker having the following structure of Formula V, wherein each n is independently 1 to 3, k is 1 to 3, and the ligand is attached to the 5′ end of the sense strand of the double-stranded RNA molecule (represented by the solid wavy line):

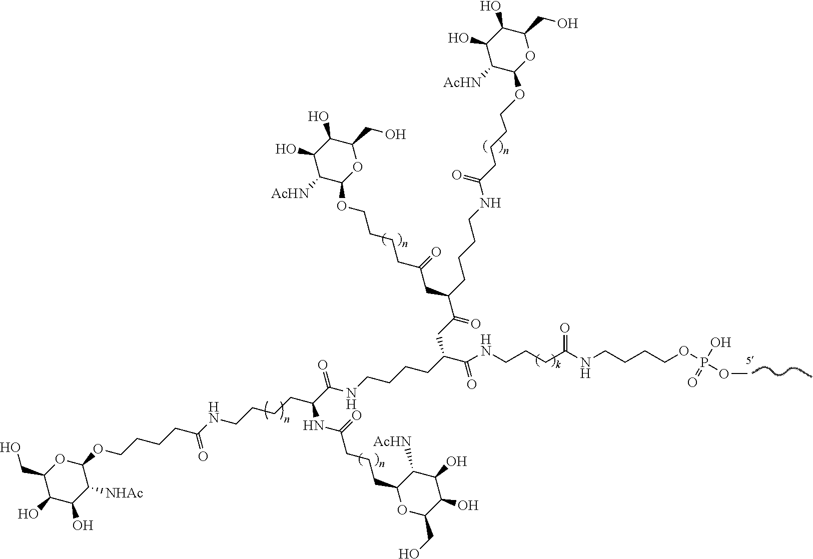

In other embodiments, the RNAi construct comprises a ligand and linker having the following structure of Formula VI, wherein each n is independently 1 to 3, k is 1 to 3, and the ligand is attached to the 5′ end of the sense strand of the double-stranded RNA molecule (represented by the solid wavy line):

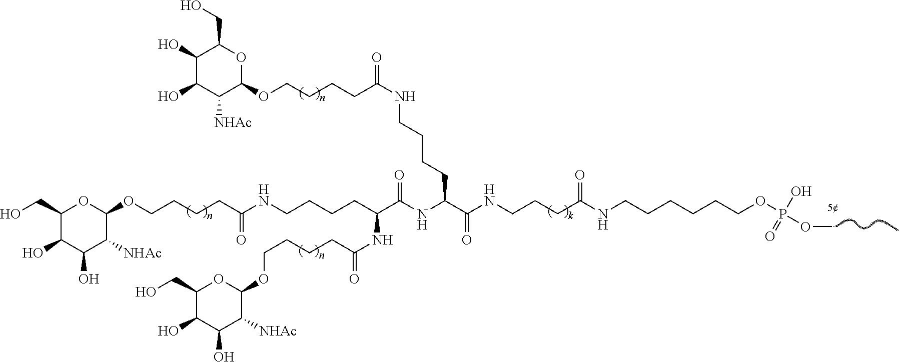

In one particular embodiment, the RNAi construct comprises a ligand and linker having the following structure of Formula VII, wherein X═O or S and wherein the ligand is attached to the 5′ end of the sense strand of the double-stranded RNA molecule (represented by the squiggly line):

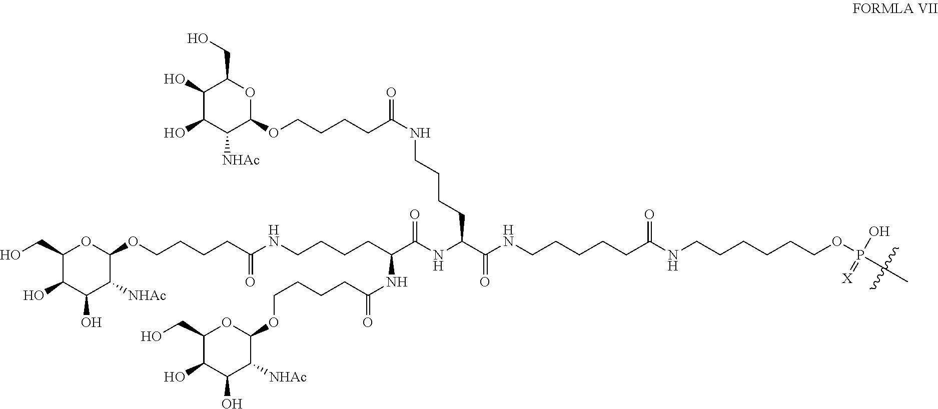

In some embodiments, the RNAi construct comprises a ligand and linker having the following structure of Formula VIII, wherein each n is independently 1 to 3 and the ligand is attached to the 5′ end of the sense strand of the double-stranded RNA molecule (represented by the solid wavy line):

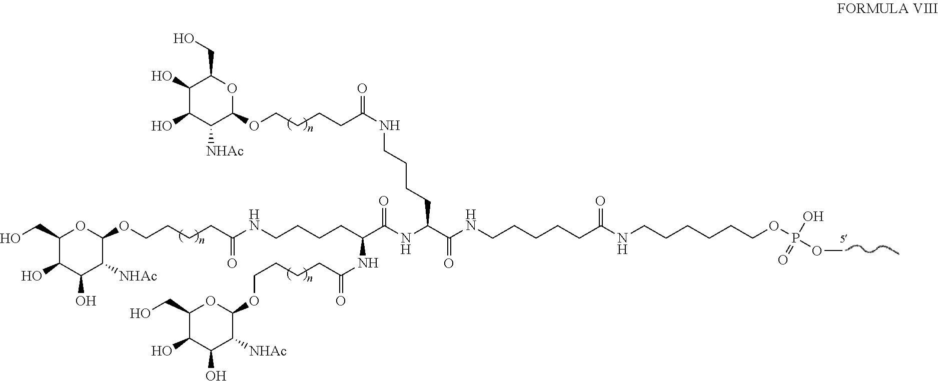

In certain embodiments, the RNAi construct comprises a ligand and linker having the following structure of Formula IX, wherein the ligand is attached to the 5′ end of the sense strand of the double-stranded RNA molecule (represented by the solid wavy line):

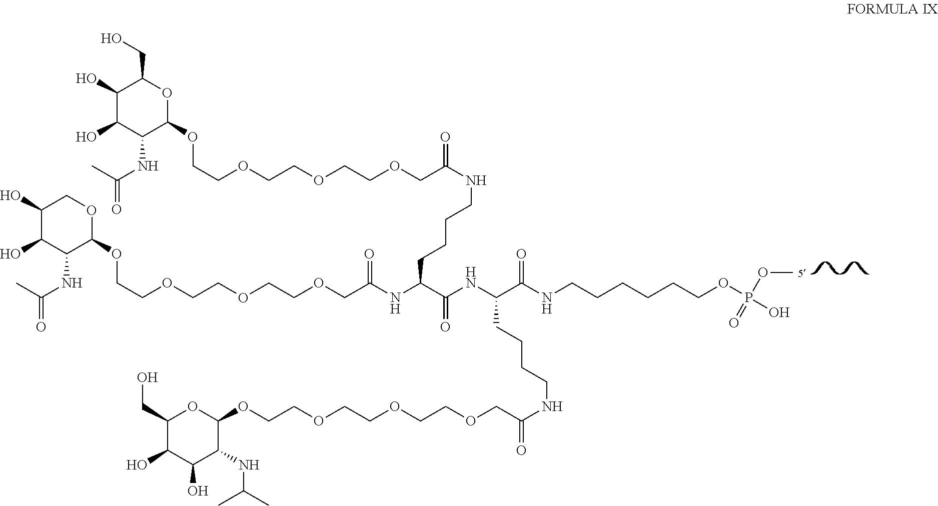 [SMILES: COP(=O)(O)OCCCCCCNC(=O)[C@H](CCCCNC(=O)COCCOCCOCCO[C@@H]1O[C@H](CO)[C@H](O)[C@H](O)[C@H]1NC(C)C)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1OC[C@H](O)[C@H](O)[C@H]1NC(C)=O]

A phosphorothioate bond can be substituted for the phosphodiester bond shown in any one of Formulas I-IX to covalently attach the ligand and linker to the nucleic acid strand.

The present invention also includes pharmaceutical compositions and formulations comprising the RNAi constructs described herein and pharmaceutically acceptable carriers, excipients, or diluents. Such compositions and formulations are useful for reducing expression of a target gene in a subject in need thereof. Where clinical applications are contemplated, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the RNAi constructs of the present invention, its use in therapeutic compositions is contemplated. Supplementary active ingredients also can be incorporated into the compositions, provided they do not inactivate the RNAi constructs of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal or intramuscular injection or infusion. In one embodiment, the pharmaceutical composition is formulated for intravenous delivery. In such an embodiment, the pharmaceutical composition may include a lipid-based delivery vehicle. In another embodiment, the pharmaceutical composition is formulated for subcutaneous delivery. In such an embodiment, the pharmaceutical composition may include a targeting ligand (e.g. GalNAc-containing or antibody-containing ligands described herein).

In some embodiments, the pharmaceutical compositions comprise an effective amount of an RNAi construct described herein. An “effective amount” is an amount sufficient to produce a beneficial or desired clinical result. In some embodiments, an effective amount is an amount sufficient to reduce target gene expression in a particular tissue or cell-type (e.g. liver or hepatocytes) of a subject.

Administration of the pharmaceutical compositions of the present invention may be via any common route so long as the target tissue is available via that route. Such routes include, but are not limited to, parenteral (e.g., subcutaneous, intramuscular, intraperitoneal or intravenous), oral, nasal, buccal, intradermal, transdermal, and sublingual routes, or by direct injection into liver tissue or delivery through the hepatic portal vein. In some embodiments, the pharmaceutical composition is administered parenterally. For instance, in certain embodiments, the pharmaceutical composition is administered intravenously. In other embodiments, the pharmaceutical composition is administered subcutaneously.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the RNAi constructs of the invention. Commercially available fat emulsions that are suitable for delivering the nucleic acids of the invention include Intralipid® (Baxter International Inc.), Liposyn® (Abbott Pharmaceuticals), Liposyn®II (Hospira), Liposyn®III (Hospira), Nutrilipid (B. Braun Medical Inc.), and other similar lipid emulsions. A preferred colloidal system for use as a delivery vehicle in vivo is a liposome (i.e., an artificial membrane vesicle). The RNAi constructs of the invention may be encapsulated within liposomes or may form complexes thereto, in particular to cationic liposomes. Alternatively, RNAi constructs of the invention may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), di stearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems are well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO03/093449.

In some embodiments, the RNAi constructs of the invention are fully encapsulated in a lipid formulation, e.g., to form a SNALP or other nucleic acid-lipid particle. As used herein, the term “SNALP” refers to a stable nucleic acid-lipid particle. SNALPs typically contain a cationic lipid, a non-cationic lipid, and a lipid that prevents aggregation of the particle (e.g., a PEG-lipid conjugate). SNALPs are exceptionally useful for systemic applications, as they exhibit extended circulation lifetimes following intravenous injection and accumulate at distal sites (e.g., sites physically separated from the administration site). The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

The pharmaceutical compositions suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like).

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition of the invention comprises or consists of a sterile saline solution and an RNAi construct described herein. In other embodiments, a pharmaceutical composition of the invention comprises or consists of an RNAi construct described herein and sterile water (e.g. water for injection, WFI). In still other embodiments, a pharmaceutical composition of the invention comprises or consists of an RNAi construct described herein and phosphate-buffered saline (PBS).

In some embodiments, the pharmaceutical compositions of the invention are packaged with or stored within a device for administration. Devices for injectable formulations include, but are not limited to, injection ports, pre-filled syringes, autoinjectors, injection pumps, on-body injectors, and injection pens. Devices for aerosolized or powder formulations include, but are not limited to, inhalers, insufflators, aspirators, and the like. Thus, the present invention includes administration devices comprising a pharmaceutical composition of the invention for treating or preventing one or more diseases or disorders.

The present invention provides a method for reducing or inhibiting expression of a target gene in a cell by contacting the cell with any one of the RNAi constructs described herein. The cell may be in vitro or in vivo. Target gene expression can be assessed by measuring the amount or level of target mRNA, target protein, or another biomarker linked to expression of the target gene. The reduction of target gene expression in cells or animals treated with an RNAi construct of the invention can be determined relative to the target gene expression in cells or animals not treated with the RNAi construct or treated with a control RNAi construct. For instance, in some embodiments, reduction or inhibition of target gene expression is assessed by (a) measuring the amount or level of target mRNA in cells treated with a RNAi construct of the invention, (b) measuring the amount or level of target mRNA in cells treated with a control RNAi construct (e.g. RNAi agent directed to a RNA molecule not expressed in the cells or a RNAi construct having a nonsense or scrambled sequence) or no construct, and (c) comparing the measured target mRNA levels from treated cells in (a) to the measured target mRNA levels from control cells in (b). The target mRNA levels in the treated cells and controls cells can be normalized to RNA levels for a control gene (e.g. 18S ribosomal RNA or housekeeping gene) prior to comparison. Target mRNA levels can be measured by a variety of methods, including Northern blot analysis, nuclease protection assays, fluorescence in situ hybridization (FISH), reverse-transcriptase (RT)-PCR, real-time RT-PCR, quantitative PCR, droplet digital PCR, and the like.

In other embodiments, reduction or inhibition of target gene expression is assessed by (a) measuring the amount or level of target protein in cells treated with a RNAi construct of the invention, (b) measuring the amount or level of target protein in cells treated with a control RNAi construct (e.g. RNAi agent directed to a RNA molecule not expressed in the cells or a RNAi construct having a nonsense or scrambled sequence) or no construct, and (c) comparing the measured target protein levels from treated cells in (a) to the measured target protein levels from control cells in (b). Methods of measuring target protein levels are known to those of skill in the art, and include Western Blots, immunoassays (e.g. ELISA), and flow cytometry.

The present invention also provides methods for reducing or inhibiting the expression of a target gene in a subject in need thereof comprising administering to the subject any one of the RNAi constructs described herein. The RNAi constructs of the invention can be used to treat or ameliorate conditions, diseases, or disorders associated with aberrant target gene expression or activity, for example, where overexpression of a gene product causes a pathological phenotype. Exemplary target genes include, but are not limited to, LPA, PNPLA3, ASGR1, F7, F12, FXI, APOCIII, APOB, APOL1, TTR, PCSK9, SCAP, KRAS, CD274, PDCD 1 , C5, ALAS1, HAO 1 , LDHA, ANGPTL3, SERPINA1, AGT, HAMP, LECT2, EGFR, VEGF, KIF 11, AT3, CTNNB1, HMGB1, HIF 1A, and STATS. Target genes may also include viral genes, such as hepatitis B and hepatitis C viral genes, human immunodeficiency viral genes, herpes viral genes, etc. In some embodiments, the target gene is a gene that encodes a human micro RNA (miRNA).

In certain embodiments, expression of the target gene is reduced in cells or a subject by at least 50% by an RNAi construct of the invention. In some embodiments, expression of the target gene is reduced in cells or a subject by at least 60%, at least 65%, at least 70%, at least 75%, at least 80%, or at least 85% by an RNAi construct of the invention. In other embodiments, the expression of a target gene is reduced in liver cells by about 90% or more, e.g., 91%, 92%, 93%, 94%, 95%, 96%, 97%, 98%, 99%, or more by an RNAi construct of the invention. The percent reduction of target gene expression can be measured by any of the methods described herein as well as others known in the art.

The following examples, including the experiments conducted and the results achieved, are provided for illustrative purposes only and are not to be construed as limiting the scope of the appended claims.

### EXAMPLES

### Example 1. In Vivo Activity of PNPLA3 RNAi Constructs with Different Chemical Modification Patterns

To evaluate the effect of different chemical modification patterns on in vivo efficacy of RNAi constructs, RNAi constructs targeting the patatin-like phospholipase domain-containing 3 (PNPLA3) gene were synthesized with various patterns of 2′-fluoro modified nucleotides and 2′-O-methyl modified nucleotides and evaluated in a humanized mouse model expressing PNPLA3 as described in detail below.

RNAi constructs were synthesized using solid phase phosphoramidite chemistry. Synthesis was performed on a MerMadel2 (Bioautomation) instrument.

### Materials

Acetonitrile (DNA Synthesis Grade, AXO152-2505, EMD)

Capping Reagent A (80:10:10 (v/v/v) tetrahydrofuran/lutidine/acetic anhydride, BIO221/4000, EMD)

Capping Reagent B (16% 1-methylimidazole/tetrahydrofuran, BIO345/4000, EMD)

Activator Solution (0.25 M 5-(ethylthio)-1H-tetrazole (ETT) in acetonitrile, BIO152/0960, EMD)

Detritylation Reagent (3% dichloroacetic acid in dichloromethane, BIO830/4000, EMD)

Oxidation Reagent (0.02 M iodine in 70:20:10 (v/v/v) tetrahydrofuran/pyridine/water, BIO420/4000, EMD)

Diethylamine solution (20% DEA in acetonitrile, NC0017-0505, EMD)

Thiolation Reagent (0.05 M 5-N-[(dimethylamino)methylene]amino-3H-1,2,4-dithiazole-3-thione (BIOSULII/160K) in 40:60 (v/v) pyridine/acetonitrile)

5′-Aminohexyl linker phosphoramidite, phosphorylating phosphoramidite, 2′-deoxythymidine phosphoramidite, and 2′-methoxy and 2′-fluoro phosphoramidites of adenosine, guanosine, cytosine, and uridine (Thermo Fisher Scientific), 0.10 M in acetonitrile over ˜10 mL of molecular sieves (3 Å, J. T. Baker)

CPG Support (Hi-Load Universal Support, 500A (BH5-3500-G1), 79.6 μmol/g, 0.126 g (10 μmol))

Ammonium hydroxide (concentrated, J. T. Baker)

### Synthesis

Reagent solutions, phosphoramidite solutions, and solvents were attached to the MerMade12 instrument. Solid support was added to each column (4 mL SPE tube with top and bottom frit), and the columns were affixed to the instrument. The columns were washed twice with acetonitrile. The phosphoramidite and reagent solution lines were purged. The synthesis was initiated using the Poseidon software. The synthesis was accomplished by repetition of the deprotection /coupling/oxidation/capping synthesis cycle. Specifically, to the solid support was added detritylation reagent to remove the 5′-dimethoxytrityl (DMT) protecting group. The solid support was washed with acetonitrile. To the support was added phosphoramidite and activator solution followed by incubation to couple the incoming nucleotide to the free 5′-hydroxyl group. The support was washed with acetonitrile. To the support was added oxidation or thiolation reagent to convert the phosphite triester to the phosphate triester or phosphorothioate. To the support was added capping reagents A and B to terminate any unreacted oligonucleotide chains. The support was washed with acetonitrile. After the final reaction cycle, the resin was washed with diethylamine solution to remove the 2-cyanoethyl protecting groups. The support was washed with acetonitrile and dried under vacuum.

### GalNAc Conjugation

Sense strands for conjugation to a trivalent N-acetyl-galactosamine (GalNAc) moiety (structure shown in Formula VII below) were prepared with a 5′-aminohexyl linker. After automated synthesis, the column was removed from the instrument and transferred to a vacuum manifold in a hood. The 5′-monomethoxytrityl (MMT) protecting group was removed from the solid support by successive treatments with 2 mL aliquots of 1% trifluoroacetic acid (TFA) in dichloromethane (DCM) with vacuum filtration. When the orange/yellow color was no longer observable in the eluent, the resin was washed with dichloromethane. The resin was washed with 5 mL of 2% diisopropylethylamine in N,N-dimethylformamide (DMF). In a separate vial a solution of GalNAc3-Lys2-Ahx (67 mg, 40 μmol) in DMF (0.5 mL), the structure and synthesis of which is described below, was prepared with 1,1,3,3-tetramethyluronium tetrafluoroborate (TATU, 12.83 mg, 40 μmol) and diisopropylethylamine (DIEA)(10.5 μL, 360 μmol). The activated coupling solution was added to the resin, and the column was capped and incubated at room temperature overnight. The resin was washed with DMF, DCM, and dried under vacuum.

### Cleavage

The synthesis columns were removed from the synthesizer or vacuum manifold. The solid support from each column was transferred to a 10 mL vial. To the solid support was added 4 mL of concentrated ammonium hydroxide. The cap was tightly affixed to the bottle, and the mixture was heated at 55° C. for 4 h. The bottle was moved to the freezer and cooled for 20 minutes before opening in the hood. The mixture was filtered through an 8 mL SPE tube to remove the solid support. The vial and solid support were rinsed with 1 mL of 50:50 ethanol/water.

### Analysis and Purification

A portion of the combined filtrate was analyzed and purified by anion exchange chromatography. The pooled fractions were desalted by size exclusion chromatography and analyzed by ion pair-reversed phase high-performance liquid chromatograph-mass spectrometry (HPLC-MS). The pooled fractions were lyophilized to obtain a white amorphous powder.

### Analytical Anion Exchange Chromatography (AEX):

Column: Thermo DNAPac PA200RS (4.6×50 mm, 4 μm)

Instrument: Agilent 1100 HPLC

Buffer A: 20 mM sodium phosphate, 10% acetonitrile, pH 8.5

Buffer B: 20 mM sodium phosphate, 10% acetonitrile, pH 8.5, 1 M sodium bromide

Flow rate: 1 mL/min at 40° C.

Gradient: 20-65% B in 6.2 min

### Preparative Anion Exchange Chromatography (AEX):

Column: Tosoh TSK Gel SuperQ-5PW, 21×150 mm, 13 μm

Instrument: Agilent 1200 HPLC

Buffer A: 20 mM sodium phosphate, 10% acetonitrile, pH 8.5

Buffer B: 20 mM sodium phosphate, 10% acetonitrile, pH 8.5, 1 M sodium bromide

Flow rate: 8 mL/min

Injection volume: 5 mL

Gradient: 35-55% B over 20 min

### Preparative Size Exclusion Chromatography (SEC):

Column: GE Hi-Prep 26/10

Instrument: GE AKTA Pure

Buffer: 20% ethanol in water

Flow Rate: 10 mL/min

Injection volume: 15 mL using sample loading pump

### Ion Pair-Reversed Phase (IP-RP) HPLC:

Column: Water Xbridge BEH OST C18, 2.5 μm, 2.1×50 mm

Instrument: Agilent 1100 HPLC

Buffer A: 15.7 mM DIEA, 50 mM hexafluoroisopropanol (HFIP) in water

Buffer B: 15.7 mM DIEA, 50 mM HFIP in 50:50 water/acetonitrile

Flow rate: 0.5 mL/min

Gradient: 10-30% B over 6 min

### Annealing

A small amount of the sense strand and the antisense strand were weighed into individual vials. To the vials was added siRNA reconstitution buffer (Qiagen) or phosphate buffered saline (PBS) to an approximate concentration of 2 mM based on the dry weight. The actual sample concentration was measured on the NanoDrop One (ssDNA, extinction coefficient=33 μg/OD260). The two strands were then mixed in an equimolar ratio, and the sample was heated for 5 minutes in a 90° C. incubator and allowed to cool slowly to room temperature. The sample was analyzed by AEX. The duplex was registered and submitted for in vivo testing as described in more detail below.

### Preparation of GalNAc3-Lys2-Ahx

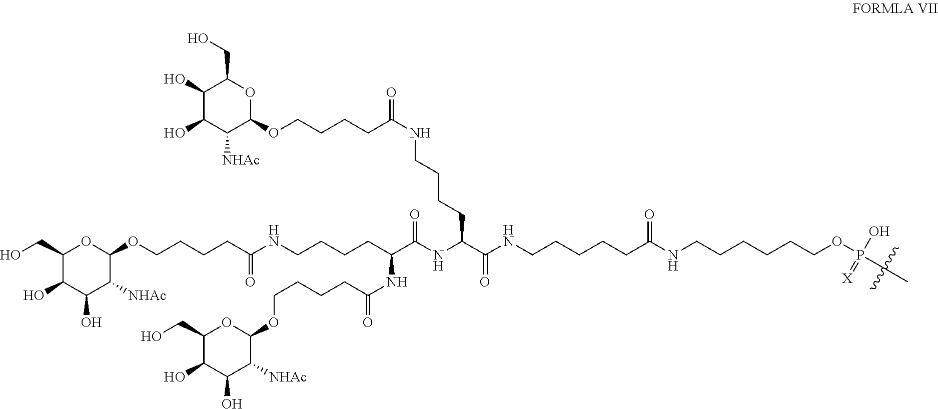

wherein X═O or S. The squiggly line represents the point of attachment to the 5′ terminal nucleotide of the sense strand of the RNAi construct.

To a 50 mL falcon tube was added Fmoc-Ahx-OH (1.13 g, 3.19 mmol) in DCM (30 mL) followed by DIEA (2.23 mL, 12.78 mmol). The solution was added to 2-Cl Trityl chloride resin (3.03 g, 4.79 mmol) in a 50 mL centrifuge tube and loaded onto a shaker for 2 h. The solvent was drained and the resin was washed with 17:2:1 DCM/MeOH/DIEA (30 ml×2), DCM (30 mL×4) and dried. The loading was determined to be 0.76 mmol/g with UV spectrophotometric detection at 290 nm.

3 g of the loaded 2-Cl Trityl resin was suspended in 20% 4-methylpiperidine in DMF (20 mL), and after 30 min the solvent was drained. The process was repeated one more time, and the resin was washed with DMF (30 mL×3) and DCM (30 mL×3).

To a solution of Fmoc-Lys(ivDde)-OH (3.45 g, 6 mmol) in DMF (20 mL) was added TATU (1.94 g, 6 mmol) followed by DIEA (1.83 mL, 10.5 mmol). The solution was then added to the above deprotected resin, and the suspension was set on a shaker overnight. The solvent was drained and the resin was washed with DMF (30 mL×3) and DCM (30 mL×3).

The resin was treated with 20% 4-methylpiperidine in DMF (15 mL) and after 10 min the solvent was drained. The process was repeated one more time and the resin was washed with DMF (15 mL×4) and DCM (15 mL×4).

To a solution of Fmoc-Lys(Fmoc)-OH (3.54 g, 6 mmol) in DMF (20 mL) was added TATU (1.94 g, 6 mmol) followed by DIEA (1.83 mL, 10.5 mmol). The solution was then added to the above deprotected resin and the suspension was set on a shaker overnight. The solvent was drained and the resin was washed with DMF (30 mL×3) and DCM (30 mL×3).

The resin was treated with 5% hydrazine in DMF (20 mL) and after 5 min, the solvent was drained. The process was repeated four more times and the resin was washed with DMF (30 mL×4) and DCM (30mL x 4).

To a solution of 5-(((2R,3R,4R,5R,6R)-3-acetamido-4,5-diacetoxy-6-(acetoxymethyl)tetrahydro-2H-pyran-2-yl)oxy)pentanoic acid (4.47 g, 10 mmol) in DMF (40 mL) was added TATU (3.22 g, 10 mmol), and the solution was stirred for 5 min. DIEA (2.96 mL, 17 mmol) was added to the solution, and the mixture was then added to the resin above. The suspension was kept at room temperature overnight and the solvent was drained. The resin was washed with DMF (3×30 mL) and DCM (3×30 mL).

The resin was treated with 1% TFA in DCM (30 mL with 3% Triisopropylsilane) and after 5 min, the solvent was drained. The process was repeated three more times, and the combined filtrate was concentrated in vacuo. The residue was triturated with diethyl ether (50 mL) and the suspension was filtered and dried to give the crude product. The crude product was purified with reverse phase chromatography and eluted with 0-20% of MeCN in water. The fractions were combined and lyophilized to give the product as a white solid.

Table 1 below depicts the positions of the modifications in the sense and antisense sequences for each of the modified PNPLA3 RNAi constructs. The nucleotide sequences are listed according to the following notations: dT, dA, dG, dC=corresponding deoxyribonucleotide; a, u, g, and c=corresponding 2′-O-methyl ribonucleotide; Af, Uf, Gf, and Cf=corresponding 2′-deoxy-2′-fluoro (“2′-fluoro”) ribonucleotide; Phos=terminal nucleotide has a monophosphate group at its 5′ end; invAb=inverted abasic nucleotide (i.e. abasic nucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand); and invdX=inverted deoxyribonucleotide (i.e. deoxyribonucleotide linked to adjacent nucleotide via a substituent at its 3′ position (a 3′-3′ linkage) when on the 3′ end of a strand or linked to adjacent nucleotide via a substituent at its 5′ position (a 5′-5′ internucleotide linkage) when on the 5′ end of a strand). Insertion of an “s” in the sequence indicates that the two adjacent nucleotides are connected by a phosphorothiodiester group (e.g. a phosphorothioate internucleotide linkage). Unless indicated otherwise, all other nucleotides are connected by 3′-5′ phosphodiester groups. All RNAi constructs were conjugated to the GalNAc moiety shown in Formula VII via the 5′ end of the sense strand. Table 1 also lists the pattern designation and the sequence family designation for each RNAi construct. The pattern designations are schematically represented in FIG. 1. If an RNAi construct has the same sequence family designation as another RNAi construct, then the two constructs have the same core sequence, but differ in chemical modification pattern.

TABLE 1
Exemplary Modified PNPLA3 RNAi Constructs

| | | Sequence | | |
|---|---|---|---|---|
| | Pattern | Family | | SEQ |
| Duplex | Desig- | Desig- | | ID |
| No. | nation | nation | Sense Sequence (5′-3′) | NO: |
| 2118 | CM1 | T2 | CfgGfcCfaAfuGfUfCfcAfcCfaGfcUfsusUf | 1 |
| 4544 | P1 | T2 | cggccaAfuGfUfCfCfaccagcususu | 2 |
| 2119 | CM1 | T3 | GfgUfcCfaGfcCfUfGfaAfcUfuCfuUfsusUf | 3 |
| 3552 | P1 | T3 | gguccaGfcCfUfGfAfacuucuususu | 4 |
| 2125 | CM1 | T5 | GfcUfuCfaUfgCfCfCfuUfcUfaCfaGfsusUf | 5 |
| 2393 | P1 | T5 | gcuucaUfgCfCfCfUfucuacagsusu | 6 |
| 2120 | CM1 | T6 | GfcGfgCfuUfcCfUfGfgGfcUfuCfuAfsusUf | 7 |
| 3464 | P1 | T6 | gcggcuUfcCfUfGfGfgcuucuasusu | 8 |
| 2121 | CM1 | T8 | GfuGfaCfaAfcGfUfAfcCfcUfuCfaUfsusUf | 9 |
| 3918 | P1 | T8 | gugacaAfcGfUfAfCfccuucaususu | 10 |
| 2124 | CM1 | T11 | GfgUfaUfgUfuCfCfUfgCfuUfcAfuGfsusUf | 11 |
| 2390 | P1 | T11 | ggsuaugUfuCfCfUfGfcuucaugsusu | 12 |
| 2370 | CM1 | T12 | GfuAfuGfuUfcCfUfGfcUfuCfaUfgCfsusUf | 13 |
| 2391 | P1 | T12 | guauguUfcCfUfGfCfuucaugcsusu | 14 |
| 2371 | CM1 | T15 | UfgUfuCfcUfgCfUfUfcAfuGfcCfcUfsusUf | 15 |
| 2392 | P1 | T15 | uguuccUfgCfUfUfCfaugcccususu | 16 |
| 2122 | CM1 | T16 | GfuUfcCfuGfcUfUfCfaUfgCfcCfuUfsusUf | 17 |
| 3465 | P1 | T16 | guuccuGfcUfUfCfAfugcccuususu | 18 |
| 2368 | CM1 | T19 | CfcUfgCfuUfcAfUfGfcCfcUfuCfuAfsusUf | 19 |
| 3467 | P1 | T19 | ccugcuUfcAfUfGfCfccuucuasusu | 20 |
| 2369 | CM1 | T23 | CfuUfcAfuGfcCfCfUfuCfuAfcAfgUfsusUf | 21 |
| 2394 | P1 | T23 | cuucauGfcCfCfUfUfcuacagususu | 22 |
| 2123 | CM1 | T24 | UfuCfaUfgCfcCfUfUfcUfaCfaGfuGfsusUf | 23 |
| 3539 | P1 | T24 | uucaugCfcCfUfUfCfuacagugsusu | 24 |
| 3558 | CM1 | T27 | AfuGfcCfcUfuCfUfAfcAfgUfgGfcCfsusUf | 25 |
| 3916 | P1 | T27 | augcccUfuCfUfAfCfaguggccsusu | 26 |
| 3540 | P1 | T5 | gcuucaUfgCfCfCfUfucuacaususu | 27 |
| 5241 | P2 | T5 | [invAb]gcuucaUfgCfCfCfUfucuacaususu | 28 |
| 5614 | P3 | T5 | cugcuucaUfgCfCfCfUfucuacas[invAb] | 29 |
| 5615 | P4 | T5 | [invAb]cugcuucaUfgCfCfCfUfucuacsasu | 30 |
| 6191 | P3 | T5.1 | cugcuucaUfgCfCfUfUfucuacas[invAb] | 31 |
| 6267 | P9 | T5.1 | cugcuucaUfgCfCfUfUfucuacas[invAb] | 31 |
| 7320 | P9 | T5.1 | cugcuucaUfgCfCfUfUfucuacas[invdA] | 32 |
| 7318 | P9 | T5.1 | cugcuucaUfgCfCfUfUfucuacas[invAb] | 31 |
| 7062 | P9 | T23 | ugcuucauGfcCfUfUfUfcuacags[invAb] | 33 |
| 8513 | P9 | T23 | ugcuucauGfcCfUfUfUfcuacags[invdA] | 34 |
| 8709 | P9 | T5.1 | cugcuucaUfgCfCfUfUfucuacas[invdT] | 35 |
| 8103 | CM2 | T5.1 | cugcuuCfaUfGfCfcuuucuacsasu | 36 |
| 8104 | CM3 | T5.1 | cugcuuCfaUfGfCfcuuucuacsasu | 36 |
| 8105 | CM4 | T5.1 | cugcuuCfaUfGfCfcuuucuacsasu | 36 |
| 7463 | P11 | T5.1 | cugcuucaUfgCfCfUfUfucuacas[invAb] | 31 |
| 7464 | P10 | T5.1 | [invAb]cugcuucaUfgCfCfUfUfucuacsasu | 37 |
| 7466 | P16 | T5.1 | cugcuucaUfgCfCfUfUfucuacas[invAb] | 31 |
| 7469 | P17 | T5.1 | cugcuucaUfgCfCfUfUfucUfaCfas[invAb] | 38 |
| 7470 | P15 | T5.1 | cugcuucaUfgCfCfUfUfucuacas[invAb] | 31 |
| 6883 | P3 | T5.1 | cugcuucaUfgCfCfUfUfucuacas[invAb] | 31 |
| 7319 | P9 | T5.1 | cugcuucaUfgCfCfUfUfucuacas[invAb] | 31 |
| 7064 | P3 | T23 | ugcuucauGfcCfUfUfUfcuacags[invAb] | 33 |
| 7576 | P11 | T23 | ugcuucauGfcCfUfUfUfcuacags[invAb] | 33 |
| 7579 | P18 | T23 | ugcuucauGfcCfUfUfUfcuacags[invAb] | 33 |
| 7580 | P12 | T23 | ugcuucauGfcCfUfUfUfcuAfcAfgs[invAb] | 39 |
| | | Sequence | | |
| | Pattern | Family | | SEQ |
| Duplex | Desig- | Desig- | | ID |
| No. | nation | nation | Antisense Sequence (5′-3′) | NO: |
| 2118 | CM1 | T2 | {Phos}asGfscUfgGfuGfgacAfuUfgGfcCfgsUfsu | 40 |
| 4544 | P1 | T2 | {Phos}asGfscUfgGfUfggacAfuUfggccgsusu | 41 |
| 2119 | CM1 | T3 | {Phos}asAfsgAfaGfuUfcagGfcUfgGfaCfcsUfsu | 42 |
| 3552 | P1 | T3 | {Phos}asAfsgAfaGfUfucagGfcUfggaccsusu | 43 |
| 2125 | CM1 | T5 | {Phos}csUfsgUfaGfaAfgggCfaUfgAfaGfcsUfsu | 44 |
| 2393 | P1 | T5 | {Phos}csUfsgUfaGfAfagggCfaUfgaagcsusu | 45 |
| 2120 | CM1 | T6 | {Phos}usAfsgAfaGfcCfcagGfaAfgCfcGfcsUfsu | 46 |
| 3464 | P1 | T6 | {Phos}usAfsgAfaGfCfccagGfaAfgccgcsusu | 47 |
| 2121 | CM1 | T8 | {Phos}asUfsgAfaGfgGfuacGfuUfgUfcAfcsUfsu | 48 |
| 3918 | P1 | T8 | {Phos}asUfsgAfaGfGfguacGfuUfgucacsusu | 49 |
| 2124 | CM1 | T11 | {Phos}csAfsuGfaAfgCfaggAfaCfaUfaCfcsUfsu | 50 |
| 2390 | P1 | T11 | {Phos}csAfsuGfaAfGfcaggAfaCfauaccsusu | 51 |
| 2370 | CM1 | T12 | {Phos}gsCfsaUfgAfaGfcagGfaAfcAfuAfcsUfsu | 52 |
| 2391 | P1 | T12 | {Phos}gsCfsaUfgAfAfgcagGfaAfcauacsusu | 53 |
| 2371 | CM1 | T15 | {Phos}asGfsgGfcAfuGfaagCfaGfgAfaCfasUfsu | 54 |
| 2392 | P1 | T15 | {Phos}asGfsgGfcAfUfgaagCfaGfgaacasusu | 55 |
| 2122 | CM1 | T16 | {Phos}asAfsgGfgCfaUfgaaGfcAfgGfaAfcsUfsu | 56 |
| 3465 | P1 | T16 | {Phos}asAfsgGfgCfAfugaaGfcAfggaacsusu | 57 |
| 2368 | CM1 | T19 | {Phos}usAfsgAfaGfgGfcauGfaAfgCfaGfgsUfsu | 58 |
| 3467 | P1 | T19 | {Phos}usAfsgAfaGfGfgcauGfaAfgcaggsusu | 59 |
| 2369 | CM1 | T23 | {Phos}asCfsuGfuAfgAfaggGfcAfuGfaAfgsUfsu | 60 |
| 2394 | P1 | T23 | {Phos}asCfsuGfuAfGfaaggGfcAfugaagsusu | 61 |
| 2123 | CM1 | T24 | {Phos}csAfscUfgUfaGfaagGfgCfaUfgAfasUfsu | 62 |
| 3539 | P1 | T24 | {Phos}csAfscUfgUfAfgaagGfgCfaugaasusu | 63 |
| 3558 | CM1 | T27 | {Phos}gsGfscCfaCfuGfuagAfaGfgGfcAfusUfsu | 64 |
| 3916 | P1 | T27 | {Phos}gsGfscCfaCfUfguagAfaGfggcaususu | 65 |
| 3540 | P1 | T5 | {Phos}asUfsgUfaGfAfagggCfaUfgaagcsusu | 66 |
| 5241 | P2 | T5 | {Phos}asUfsgUfaGfAfagggCfaUfgaagcsusu | 66 |
| 5614 | P3 | T5 | {Phos}asUfsgUfaGfAfagggCfaUfgaagcagsusu | 67 |
| 5615 | P4 | T5 | {Phos}asUfsgUfaGfAfagggCfaUfgaagcagsusu | 67 |
| 6191 | P3 | T5.1 | {Phos}asUfsgUfaGfAfaaggCfaUfgaagcagsusu | 68 |
| 6267 | P9 | T5.1 | {Phos}asUfsguagAfaaggCfaUfgaagcagsusu | 69 |
| 7320 | P9 | T5.1 | usUfsguagAfaaggCfaUfgaagcagsusu | 70 |
| 7318 | P9 | T5.1 | asUfsguagAfaaggCfaUfgaagcagsusu | 71 |
| 7062 | P9 | T23 | asCfsuguaGfaaagGfcAfugaagcasusu | 72 |
| 8513 | P9 | T23 | usCfsuguaGfaaagGfcAfugaagcasusu | 73 |
| 8709 | P9 | T5.1 | asUfsguagAfaaggCfaUfgaagcagsusu | 71 |
| 8103 | CM2 | T5.1 | asUfsguaGfaaAfggcaUfgAfagcagsusu | 74 |
| 8104 | CM3 | T5.1 | asUfsguaGfaaaggcaUfgAfagcagsusu | 75 |
| 8105 | CM4 | T5.1 | asUfsguaGfaAfAfggcaUfgAfagcagsusu | 76 |
| 7463 | P11 | T5.1 | asUfsgUfagAfaaggCfaUfgaagcagsusu | 77 |
| 7464 | P10 | T5.1 | asUfsguagAfaaggCfaUfgaagcagsusu | 71 |
| 7466 | P16 | T5.1 | asUfsguagAfaaGfgCfaUfgaagcagsusu | 78 |
| 7469 | P17 | T5.1 | asUfsguagAfaaGfgCfaUfgaagcagsusu | 78 |
| 7470 | P15 | T5.1 | asUfsgUfaGfAfaaGfgCfaUfgaagcagsusu | 79 |
| 6883 | P3 | T5.1 | asUfsgUfaGfAfaaggCfaUfgaagcagsusu | 80 |
| 7319 | P9 | T5.1 | usUfsguagAfaaggCfaUfgaagcagsusu | 70 |
| 7064 | P3 | T23 | asCfsuGfuAfGfaaagGfcAfugaagcasusu | 71 |
| 7576 | P11 | T23 | asCfsuGfuaGfaaagGfcAfugaagcasusu | 72 |
| 7579 | P18 | T23 | asCfsuGfuaGfaaAfgGfcAfugaagcasusu | 73 |
| 7580 | P12 | T23 | asCfsuGfuaGfaaagGfcAfugaagcasusu | 72 |

In an initial set of experiments, thirteen different PNPLA3 RNAi constructs with different sequences were synthesized to have the P1 chemical modification pattern or the CM1 control chemical modification pattern. siRNA molecules having the CM1 control chemical modification pattern have been reported to have potent and prolonged gene silencing effects in vivo. See Nair et al., J. Am. Chem. Soc., Vol. 136:16958-16961, 2014. The efficacy of the chemically modified RNAi constructs in inhibiting PNPLA3 gene expression was evaluated in a humanized mouse model expressing wild-type human PNPLA3 or variant forms of human PNPLA3. To create the mouse model, associated adenovirus (AAV; serotype AAV8 or AAV7; endotoxin-free) diluted in phosphate buffered saline (Thermo Fisher Scientific,14190-136) to 1×10¹² viral particles per animal, was injected intravenously into the tail vein of C57BL/6NCrl male mice (Charles River Laboratories Inc.) to drive expression of human PNPLA3, PNPLA3^{rs738409}, or PNPLA3^{rs738409-rs738408} genes. Mice were generally 10-12 weeks of age and an n of 4 to 6 animals were included per treatment group.

All RNAi constructs were tested in mice injected with AAV-PNPLA3, PNPLA3^{rs738409}, and/or PNPLA3^{rs738409-rs738408}. At least two vehicle-treated control groups: AAV-empty vector and AAV-PNPLA3, PNPLA3^{rs738409}, or PNPLA3^{rs738409-rs738408} treated with vehicle were also included. Two weeks post-AAV injection, mice were treated with a single dose of RNAi construct (0.5 mM), via subcutaneous injection, at 0.5, 1.0, 3.0 or 5.0 milligrams per kilogram of animal, diluted in phosphate buffered saline (Thermo Fisher Scientific,14190-136). At 8, 15, 22, 28 or 42 days post-RNAi construct injection, livers were collected from the animals, snap frozen in liquid nitrogen, processed for purified RNA using a Qiagen QIACube HT instrument (9001793) and a Qiagen RNeasy 96 QIACube HT Kit (74171) according to manufacturer's instructions. Samples were analyzed using a QIAxpert system (9002340). RNA was treated with Promgea RQ1 RNase-Free DNase (M6101) and prepared for Real-Time qPCR using the Applied Biosystem TaqManTM RNA-to-CTTM 1-Step kit (4392653). Real-Time qPCR was run on a QuantStudio Real-Time PCR machine. Results are based on gene expression of human PNPLA3 as normalized to mouse Gapdh (TaqMan™ assays from Invitrogen, hs00228747_m1 and 4352932E, respectively), and presented as the relative knockdown of human PNPLA3 mRNA expression compared to vehicle-treated control animals.

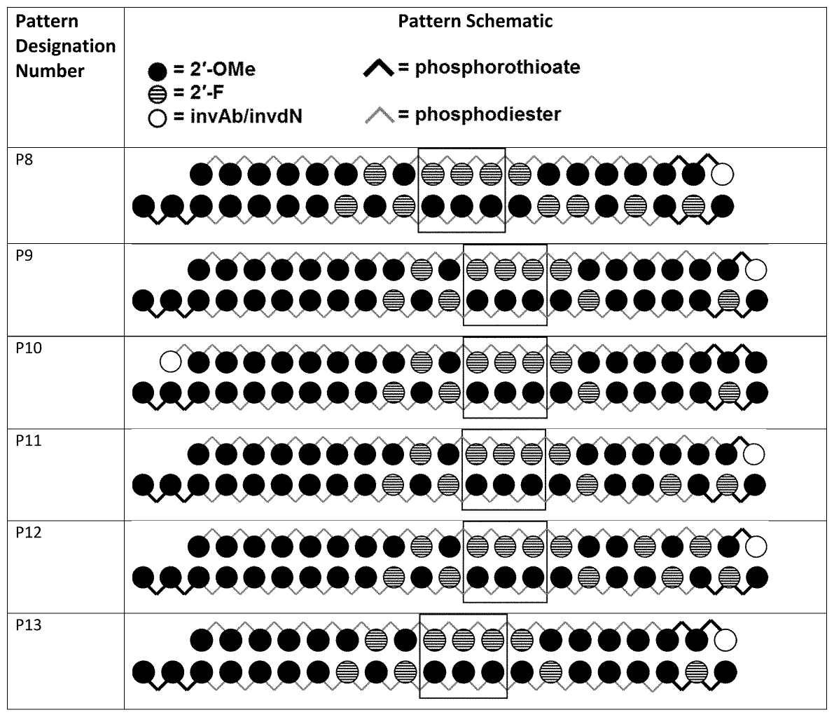

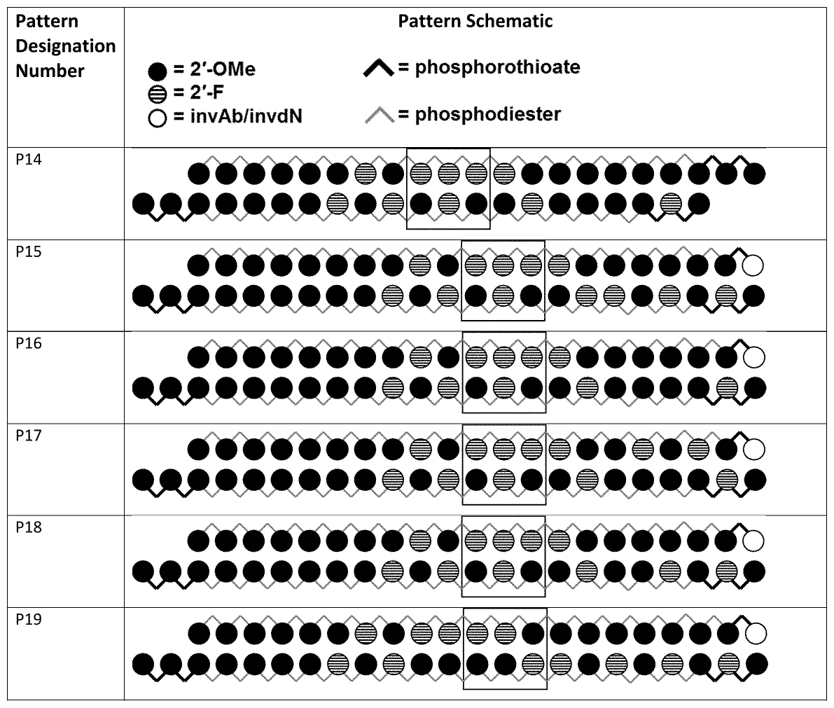

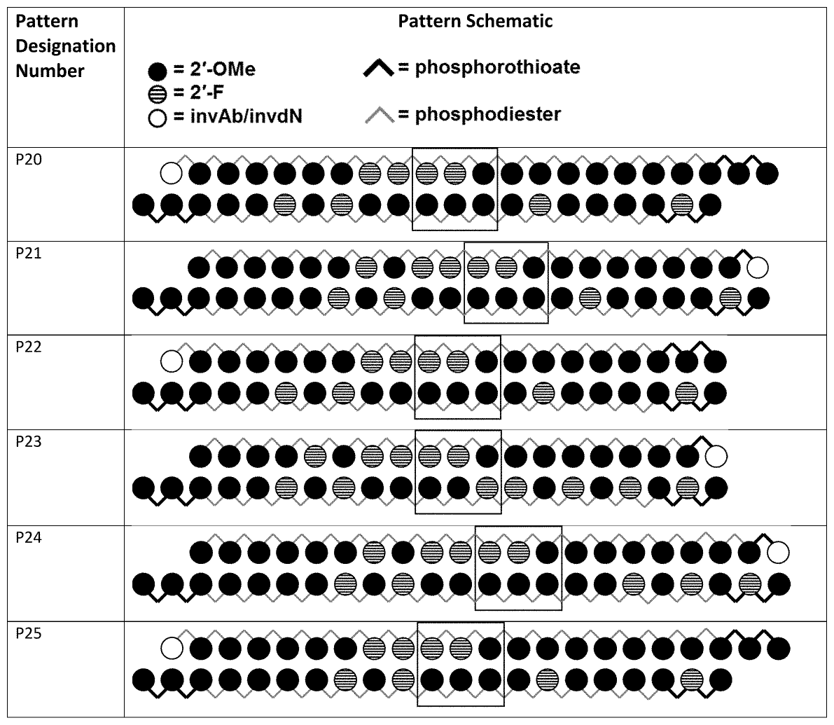

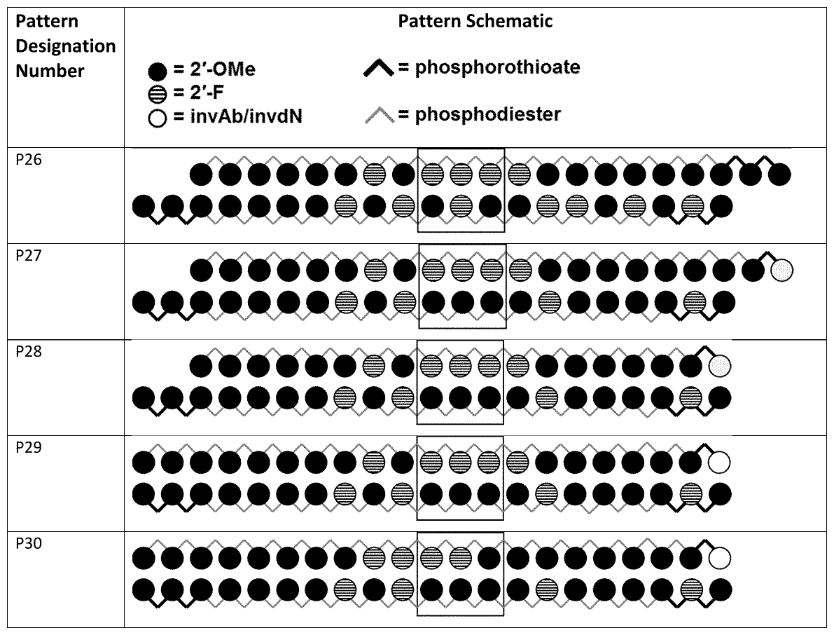

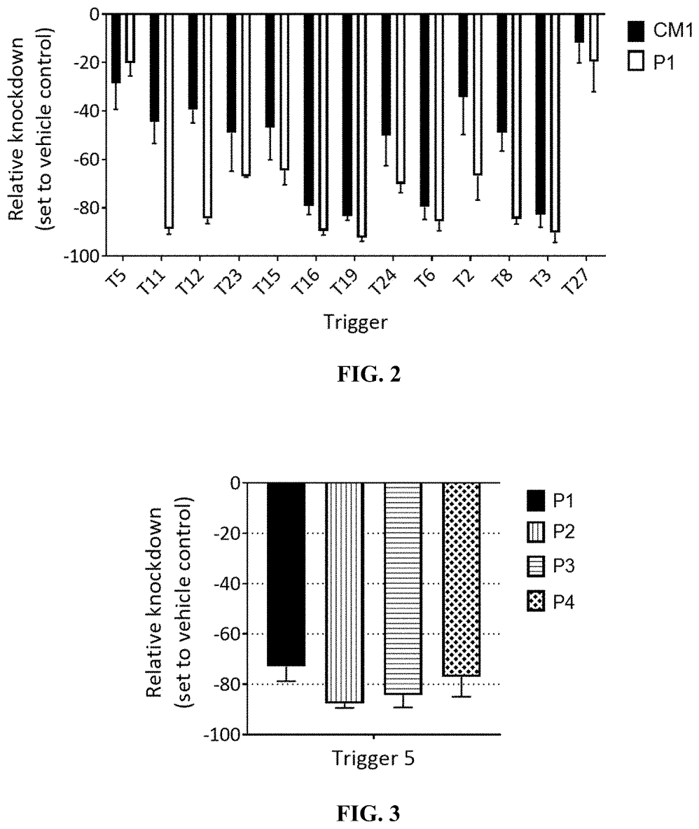

The results from this initial set of experiments comparing RNAi constructs with a P1 chemical modification pattern (duplex nos. 4544, 3552, 2393, 3464, 3918, 2390, 2391, 2392, 3465, 3467, 2394, 3539, and 3916) to those with the CM1 control modification pattern (duplex nos. 2118, 2119, 2125, 2120, 2121, 2124, 2370, 2371, 2122, 2368, 2369, 2123, and 3558) are shown in FIG. 2. When the RNAi constructs were subcutaneously administered at 5 mg/kg to mice expressing the human PNPLA3^{rs738409} variant gene, the constructs having the P1 pattern generally reduced PNPLA3 expression to a greater degree when measured 8 days following injection than the constructs having the CM1 pattern regardless of sequence.

Variations of the P1 modification pattern to modify the length of the strands, the nature of the ends of the RNAi construct (i.e. overhang versus blunt end), and/or to include inverted abasic nucleotides at the 5′ or 3′ end of the sense strand, were made and applied to RNAi constructs having the same core sequence. The RNAi constructs with the new patterns were evaluated in the humanized mouse model for improvements in in vivo efficacy. Specifically, RNAi constructs with the P1, P2, P3, or P4 chemical modification patterns (duplex nos. 3540, 5241, 5614, and 5615) were administered subcutaneously to mice expressing the human PNPLA3^{rs738409} variant gene at a dose of 5 mg/kg. Expression levels of human PNPLA3 in the liver were assessed at 15 days following administration of the RNAi constructs. The results are shown in FIG. 3. RNAi constructs with the P2, P3, or P4 patterns produced a greater average reduction of PNPLA3 expression than RNAi constructs with the P1 pattern.

Further variations of the P3 pattern were made to increase the potency and duration of mRNA knockdown in vivo. The 2′-fluoro modified nucleotides at positions 4 and 6 in the antisense strand counting from the 5′ end in the P3 pattern (duplex no. 6191) were changed to 2′-O-methyl modified nucleotides to produce the P9 pattern. (duplex no. 6267). An RNAi construct having the P9 pattern with an inverted adenosine deoxyribonucleotide in place of the inverted abasic nucleotide at the 3′ end of the sense strand (duplex no. 7320) was also synthesized. All three constructs were evaluated in the humanized mouse model described above. In animals treated with 5 mg/kg of duplex no. 6267, human PNPLA3 liver expression was reduced by 97% at 22 days following administration, whereas animals treated with 5 mg/kg of duplex no. 6191 exhibited a 92% reduction in human PNPLA3 liver expression levels at the same time point. Duplex no. 7320 was more potent and produced a longer duration of gene knockdown than duplex nos. 6191 and 6267 as animals treated with 3 mg/kg of duplex no. 7320 exhibited a 95% reduction in human PNPLA3 liver expression levels at 28 days following administration.

The P9 pattern was applied to PNPLA3 RNAi constructs with two different core sequences (duplex nos. 7318, 7320, 7062, 8513, and 8709) and evaluated for in vivo efficacy in an in vivo bioluminescence imaging assay at doses of 1 mg/kg and 3 mg/kg. For the bioluminescence imaging assay, an associated adenovirus (AAV) vector was designed to contain the murine cytomegalovirus promoter, the full sequence for Firefly Luciferase, and then, immediately downstream from the Firefly Luciferase stop codon, a synthesized string of mRNA sequences specific to the RNAi constructs to be tested. The mRNA sequences were flanked by ten additional nucleotides on each end. The vector, “PP3A (DM),” was packaged into AAV serotype, AAVDJ8 (endotoxin-free). Prior to injection, PP3A (DM) was diluted in phosphate buffered saline (Thermo Fisher Scientific,14190-136) to 5×10¹¹ viral particles per animal and injected intravenously into the tail vein of BALB/c male mice (Charles River Laboratories Inc.). Mice were generally 10-12 weeks of age and an n=5 animals were included per group.

Two weeks after AAV injection, mice were injected with RediJect D-Luciferin Bioluminescent Substrate (PerkinElmer, 770504) according to manufacturer's instructions. After a ten-minute pulse, mice were imaged on an IVIS Spectrum In Vivo Imaging System (PerkinElmer). Mice were then randomized into groups according to baseline total flux scores from a defined region of interest encompassing the liver. Once randomized, mice were treated with a single dose of RNAi construct (0.5 mM), via subcutaneous injection, at 1.0 or 3.0 milligrams per kilogram of body weight, diluted in phosphate buffered saline (Thermo Fisher Scientific, 14190-136), or treated with phosphate buffered saline only (indicated as “vehicle”). Mice were imaged weekly following the same protocol, applying the same gating constraints for total flux scores. Data is represented as total flux (photons per second, y-axis) versus the week post-RNAi construct injection (x-axis). A reduction in total flux indicates reduced expression of the luciferase reporter.

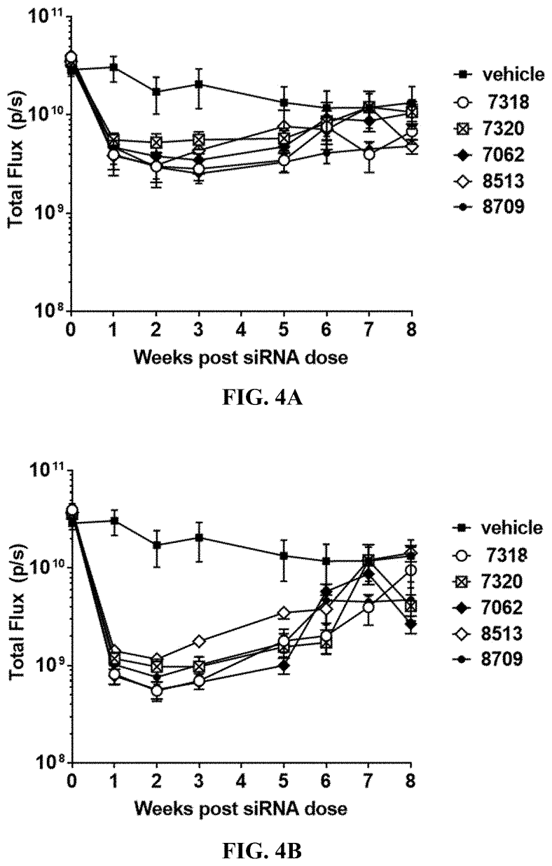

The results of this experiment are shown in FIGS. 4A and 4B. The signal from the luciferase reporter from animals treated with the different RNAi constructs having the P9 pattern was significantly reduced as compared to the signal from vehicle-treated animals for at least 3 weeks following a single dose of 1 mg/kg (FIG. 4A) and at least 5 weeks for a single dose of 3 mg/kg (FIG. 4B) of the RNAi constructs. For many of the RNAi constructs, a single 3 mg/kg dose was sufficient to inhibit luciferase reporter expression for up to 6 weeks.

These RNAi constructs (duplex nos. 7318, 7320, 7062, 8513, and 8709) were also evaluated in the humanized mouse model described above. Specifically, the RNAi constructs were administered subcutaneously to mice expressing the humanized PNPLA3^{rs738409-rs738408} variant gene at 0.5, 1, or 3 mg/kg. Expression levels of human PNPLA3 in the liver were assessed by qPCR at 28 or 42 days following administration of the RNAi constructs. The results are presented as the relative knockdown of human PNPLA3 mRNA expression compared to vehicle-treated control animals and are shown in Table 2 below.

TABLE 2
In Vivo Efficacy of PNPLA3 RNAi Constructs

| | | Day 28 | | Day 42 | |
|---|---|---|---|---|---|
| | | Average | | Average | |
| | | Relative | | Relative | |
| Duplex | Dose | Knockdown | Standard | Knockdown | Standard |
| No. | (mg/kg) | (n = 4) | Error | (n = 4) | Error |
| 8709 | 0.5 | −57.95 | 2.07 | — | — |
| 8513 | 0.5 | −46.58 | 7.95 | — | — |
| 7318 | 0.5 | −68.83 | 6.18 | — | — |
| 7320 | 0.5 | −50.01 | 3.38 | — | — |
| 7062 | 0.5 | −50.65 | 5.23 | — | — |
| 8709 | 1 | −71.96 | 4.58 | −54.55 | 5.67 |
| 8513 | 1 | −74.47 | 2.17 | −60.54 | 2.32 |
| 7318 | 1 | −76.88 | 3.04 | −71.07 | 2.41 |
| 7320 | 1 | −66.30 | 3.27 | −62.21 | 4.36 |
| 7062 | 1 | −69.37 | 2.32 | −57.74 | 4.30 |
| 8709 | 3 | −91.70 | 2.00 | −81.13 | 3.19 |
| 8513 | 3 | −90.54 | 1.17 | −74.77 | 3.28 |
| 7318 | 3 | −93.25 | 1.13 | −70.80 | 8.38 |
| 7320 | 3 | −91.08 | 0.60 | −80.12 | 4.64 |
| 7062 | 3 | −90.52 | 1.42 | −83.75 | 1.56 |

RNAi constructs having the P9 modification pattern are more potent and produce a longer duration of gene knockdown than previously tested patterns. Administration of the RNAi constructs at a single dose of 0.5 mg/kg resulted in about 50% reduction in human PNPLA3 liver expression at four weeks after administration of the single dose, whereas administration of the constructs at a dose of 1 mg/kg resulted in about 70% reduction in human PNPLA3 liver expression at four weeks after administration of the single dose. The 1 mg/kg dose was sufficient to maintain greater than 55% reduction of PNPLA3 expression out to six weeks after a single dose. Administration of a single dose of 3 mg/kg of the RNAi constructs resulted in a 90% or greater reduction of liver expression of human PNPLA3 at four weeks following administration of the single dose. Liver expression of human PNPLA3 was still reduced to about 75% or greater at six weeks following administration of the 3 mg/kg dose. The improved potency and duration of gene knockdown were observed with RNAi constructs having two distinct sequences illustrating that the P9 chemical modification pattern is effective in stabilizing RNAi constructs at least partially independent of nucleobase sequence.

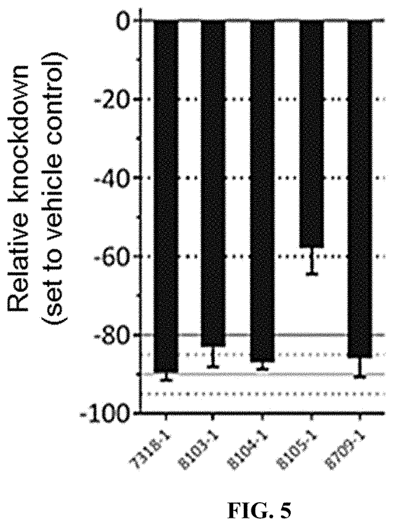

Next, the in vivo efficacy of PNPLA3 RNAi constructs having the P9 chemical modification pattern were compared to PNPLA3 RNAi constructs having one of three different control modification patterns. The CM2, CM3, and CM4 modification patterns have been previously reported to increase the metabolic stability of siRNA molecules leading to improved potency and duration of gene silencing. See Foster et al., Molecular Therapy, Vol. 26: 708-717, 2018. All the RNAi constructs had the same core nucleotide sequences in the sense and antisense strands and differed only in the chemical modification pattern. Two different constructs having the P9 modification pattern were synthesized—one having an inverted abasic at the 3′ end of the sense strand (duplex no. 7318) and one having an inverted deoxythymidine at the 3′ end of the sense strand (duplex no. 8709). RNAi constructs having one of the CM2, CM3, or CM4 modification patterns were also synthesized (duplex nos. 8103, 8104, and 8105, respectively). Each of the RNAi constructs were then administered subcutaneously to mice expressing the humanized PNPLA3^{rs738409-rs738408} variant gene at a dose of 3 mg/kg. Expression levels of human PNPLA3 in the liver were assessed by qPCR at 28 days following administration of the RNAi constructs. The results are shown in FIG. 5. The RNAi construct having the P9 modification pattern with the inverted abasic at the 3′ end of the sense strand (duplex no. 7318) produced the greatest reduction in liver PNPLA3 expression among all constructs tested. The RNAi construct having the P9 modification pattern with the inverted deoxythymidine at the 3′ end of the sense strand (duplex no. 8709) produced a greater reduction in liver PNPLA3 expression than the construct having the CM4 pattern (duplex no. 8105) and comparable reductions in liver PNPLA3 expression to the constructs having the CM2 and CM3 patterns (duplex nos. 8103 and 8104, respectively).

In a separate set of experiments, alternative variations of the P3 modification pattern were designed and evaluated for in vivo efficacy in the humanized PNPLA3 mouse model. The variations of the P3 pattern were applied to RNAi constructs with two different sequences. The sequences of the sense and antisense strands for each of the RNAi constructs are shown in Table 1 and the modification patterns are shown schematically in FIG. 1. The RNAi constructs were administered subcutaneously to mice expressing the humanized PNPLA3^{rs738409-rs738408} variant gene at a dose of 3 mg/kg. Expression levels of human PNPLA3 in the liver were assessed by qPCR at 28 days following administration of the RNAi constructs. The results are shown in Table 3 below. All the RNAi constructs produced about a 90% or greater reduction in liver expression of human PNPLA3 at four weeks following a single subcutaneous injection of 3 mg/kg.

TABLE 3
In Vivo Efficacy of PNPLA3 RNAi Constructs with
Alternative Chemical Modification Patterns

| | | | Average | |
|---|---|---|---|---|
| | | | Relative | |
| | | Sequence | Knockdown | |
| Duplex | Pattern | Family | at day 28 | Standard |
| No. | Designation | Designation | (n = 4) | Error |
| 6883 | P3 | T5.1 | −89.28 | 2.29 |
| 7319 | P9 | T5.1 | −95.93 | 0.91 |
| 7464 | P10 | T5.1 | −90.63 | 1.52 |
| 7463 | P11 | T5.1 | −93.78 | 0.90 |
| 7470 | P15 | T5.1 | −90.58 | 2.70 |
| 7466 | P16 | T5.1 | −95.25 | 0.26 |
| 7469 | P17 | T5.1 | −95.43 | 0.78 |
| 7064 | P3 | T23 | −95.67 | 1.42 |
| 7576 | P11 | T23 | −89.20 | 1.90 |
| 7580 | P12 | T23 | −93.68 | 1.29 |
| 7579 | P18 | T23 | −91.35 | 1.84 |

### Example 2. In Vivo Activity of ASGR1 RNAi Constructs with Different Chemical Modification Patterns

As shown in Example 1, the P1 chemical modification pattern applied to 13 different RNAi constructs with different sequences targeting human PNPLA3 mRNA improved the gene silencing potency of the constructs. To explore whether the P1 chemical modification pattern would enhance the potency of RNAi constructs targeting another liver gene, an RNAi construct targeting the asialoglycoprotein receptor 1 (ASGR1) mRNA was synthesized with the P1 chemical modification pattern according to the methods described in Example 1. An RNAi construct having the same sequence was synthesized with the CM1 control chemical modification pattern. The sequences of the RNAi constructs are provided below in Table 4 using the same notations described above for Table 1. A GalNAc moiety with the structure shown in Formula VII was conjugated to the 5′ end of the sense strand of the RNAi construct designated as duplex no. 1520 and a GalNAc moiety with the structure shown in Formula IX was conjugated to the 5′ end of the sense strand of the RNAi construct designated as duplex no. 1421. Conjugation of the GalNAc moieties to the sense strands of the RNAi constructs was conducted as described in Example 1, except that for the GalNAc moiety with the structure shown in Formula IX, the GalNAc moiety was prepared as follows. To a solution of 2-(2-(2-(2-(((2R,3R,4R, 5R,6R)-3-acetamido-4,5-diacetoxy-6-(acetoxymethyl)tetrahydro-2H-pyran-2-yl)oxy)ethoxy)ethoxy)ethoxy)acetic acid (5.37 g, 10 mmol) in DMF (40 mL) was added TATU (3.22 g, 10 mmol), and the solution was stirred for 5 min. DIEA (2.96 mL, 17 mmol) was added to the solution, and the mixture was then added to the resin described in Example 1 above. The suspension was kept at room temperature overnight and the solvent was drained. The resin was washed with DMF (3×30 mL) and DCM (3×30 mL).

TABLE 4
Exemplary Modified ASGR1 RNAi Constructs

| | Pattern | | | SEQ |
|---|---|---|---|---|
| Duplex | Desig- | GalNAc | | ID |
| No. | nation | Moiety | Sense Sequence (5′-3′) | NO: |
| 1421 | CM1 | Formula IX | GfuGfgGfaAfgAfAfAfgAfuGfaAfgUfsusUf | 81 |
| 1520 | P1 | Formula VII | gugggaAfgAfAfAfGfaugaagususu | 82 |
| | Pattern | | | SEQ |
| Duplex | Desig- | GalNAc | | ID |
| No. | nation | Moiety | Antisense Sequence (5′-3′) | NO: |
| 1421 | CM1 | Formula IX | {Phos}asCfsuUfcAfuCfuuuCfuUfcCfcAfcsUfsu | 83 |
| 1520 | P1 | Formula VII | {Phos}asCfsuUfcAfUfcuuuCfuUfcccacsusu | 84 |

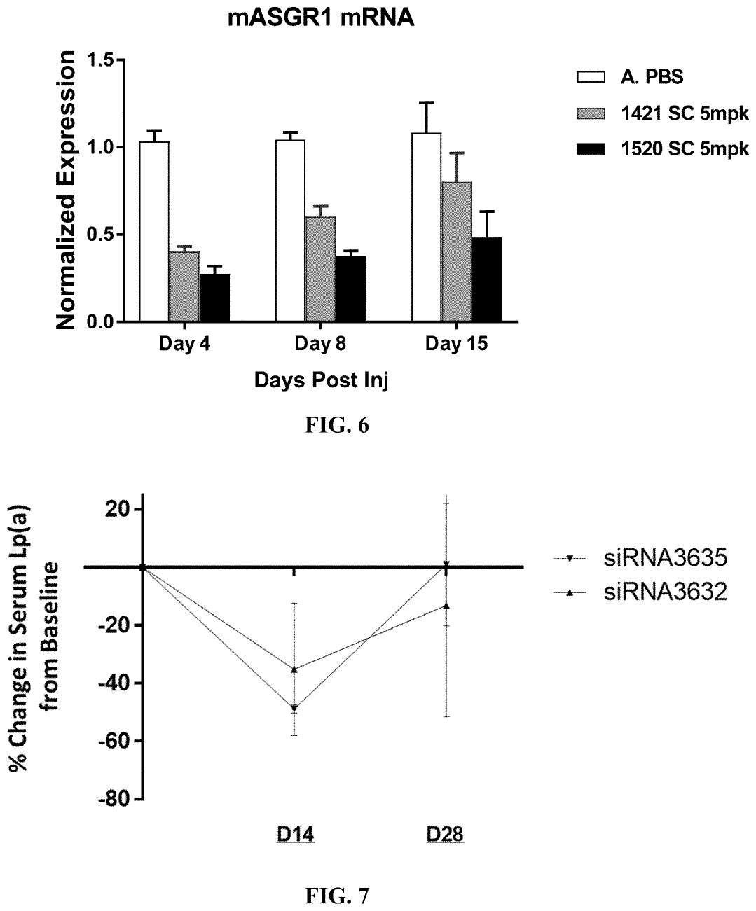

The in vivo efficacy of the RNAi constructs in inhibiting liver mouse ASGR1 expression was evaluated by administering the RNAi constructs to C57BL/6J mice. 10-12 week old wild-type C57BL/6 animals (Charles River) were fed standard chow (2020×Teklad global soy protein-free extruded rodent diet; Harlan). Mice received a subcutaneous injection of buffer or the indicated RNAi construct at 5 mg/kg body weight in 0.25 ml buffer on day 0 (n=9 per group). Three animals at day 4, three animals at day 8, and three animals at day 15 were harvested for further analysis. Liver total RNA from harvested animals was processed for qPCR analysis. The efficacy of the RNAi construct was assessed by comparing the amount of Asgr1 mRNA in liver tissue of the RNAi construct-treated animals to the amount of Asgr1 mRNA in liver tissue of animals injected with buffer. The results show that animals receiving the RNAi construct having the P1 modification pattern (duplex no. 1520) exhibited a greater reduction in liver ASGR1 expression than animals receiving the RNAi construct having the CM1 control modification pattern at all time points measured (FIG. 6). Similar to the results described in Example 1 with RNAi constructs targeting the human PNPLA3 mRNA, the P1 chemical modification pattern improves the potency of the RNAi constructs.

### Example 3. In Vivo Activity of LPA RNAi Constructs with Different Chemical Modification Patterns

To further evaluate the capability of the chemical modification patterns described herein to improve the in vivo potency of RNAi constructs, RNAi constructs targeting a third liver gene, the LPA gene, were synthesized and conjugated to a GalNAc moiety with the structure shown in Formula VII according to the methods described in Example 1. The sequences of the RNAi constructs are provided below in Table 5 using the same notations described above for Table 1. Table 5 also lists the pattern designation and the sequence family designation for each RNAi construct. The pattern designations are schematically represented in FIG. 1. If an RNAi construct has the same sequence family designation as another RNAi construct, then the two constructs have the same core sequence, but differ in chemical modification pattern.

TABLE 5
Exemplary Modified LPA RNAi Constructs

| | | Sequence | | |
|---|---|---|---|---|
| | Pattern | Family | | SEQ |
| Duplex | Desig- | Desig- | | ID |
| No. | nation | nation | Sense Sequence (5′-3′) | NO: |
| 3632 | CM1 | T101 | GfcCfcCfuUfaUfUfGfuUfaUfaCfgAfsusUf | 85 |
| 3635 | P1 | T101 | gccccuUfaUfUfGfUfuauacgasusu | 86 |
| 4973 | P1 | T102 | acacaaUfgCfUfCfAfgacgcagsusu | 87 |
| 6248 | P4 | T102 | [invAb]ugacacaaUfgCfUfCfAfgacgcsasg | 88 |
| 7934 | P19 | T102 | ugacacAfaUfGfCfUfcagacgcas[invAb] | 89 |
| 10927 | P27 | T102 | acacaaUfgCfUfCfAfgacgcaaus[invAb] | 90 |
| 11351 | P28 | T102 | acacaaUfgCfUfCfAfgacgcas[invAb] | 91 |
| 4601 | P1 | T103 | ccuagaGfgCfUfCfCfuucugaasusu | 92 |
| 6247 | P6 | T103 | agccuagaGfgCfUfCfCfuucugsasa | 93 |
| 8336 | P10 | T104 | [invAb]uucgcccuUfgGfUfGfUfuacacscsa | 94 |
| 11313 | P25 | T104 | [invAb]cgcccuUfGfGfUfguuacaccasusu | 95 |
| 11318 | P22 | T104 | [invAb]cgcccuUfGfGfUfguuacacscsa | 96 |
| 11372 | P19 | T105 | cagaauCfaAfGfUfGfuccuugcas[invAb] | 97 |
| 17183 | P25 | T105 | [invAb]gaaucaAfGfUfGfuccuugcaasusu | 98 |
| 18444 | P30 | T105 | cagaaucaAfGfUfGfuccuugcas[invAb] | 99 |
| 11580 | P27 | T106 | aaucaaGfuGfUfCfCfuugcaauus[invAb] | 100 |
| 18436 | P29 | T106 | agaaucaaGfuGfUfCfCfuugcaas[invAb] | 101 |
| 8395 | P19 | T107 | agucuuGfgUfCfCfUfcuaugacas[invAb] | 102 |
| 8401 | P9 | T107 | agucuuggUfcCfUfCfUfaugacas[invAb] | 103 |
| 11344 | P24 | T107 | agucuuGfgUfCfCfUfcuaugacas[invAb] | 102 |
| 4995 | P1 | T108 | uucugaAfgAfAfGfCfaccaacususu | 104 |
| 6182 | P2 | T108 | [invAb]uucugaAfgAfAfGfCfaccaacususu | 105 |
| 6150 | P7 | T108 | [invAb]ccuucugaAfgAfAfGfCfaccaacs[invAb] | 106 |
| | | Sequence | | |
| | Pattern | Family | | SEQ |
| Duplex | Desig- | Desig- | | ID |
| No. | nation | nation | Antisense Sequence (5′-3′) | NO: |
| 3632 | CM1 | T101 | {Phos}usCfsgUfaUfaAfcaaUfaAfgGfgGfcsUfsu | 107 |
| 3635 | P1 | T101 | {Phos}usCfsgUfaUfAfacaaUfaAfggggcsusu | 108 |
| 4973 | P1 | T102 | {Phos}csUfsgCfgUfCfugagCfaUfugugususu | 109 |
| 6248 | P4 | T102 | {Phos}csUfsgCfgUfCfugagCfaUfugugucasusu | 110 |
| 7934 | P19 | T102 | usUfsgCfgUfcUfGfagcaUfuGfugucasusu | 111 |
| 10927 | P27 | T102 | usUfsgcguCfugagCfaUfugugususu | 112 |
| 11351 | P28 | T102 | usUfsgcguCfugagCfaUfugugususu | 112 |
| 4601 | P1 | T103 | {Phos}usUfscAfgAfAfggagCfcUfcuaggsusu | 113 |
| 6247 | P6 | T103 | {Phos}usUfscAfgAfAfggagCfcUfcuaggcususu | 114 |
| 8336 | P10 | T104 | usGfsguguAfacacCfaAfgggcgaasusu | 115 |
| 11313 | P25 | T104 | usGfsguguAfacacCfaAfgggcgsusu | 116 |
| 11318 | P22 | T104 | usGfsguguAfacaccaAfgGfgcgsusu | 117 |
| 11372 | P19 | T105 | usUfsgCfaAfgGfAfcacuUfgAfuucugsusu | 118 |
| 17183 | P25 | T105 | usUfsgcaaGfgacaCfuUfgauucsusu | 119 |
| 18444 | P30 | T105 | usUfsgcaaGfgacaCfuUfgauucsusg | 120 |
| 11580 | P27 | T106 | asUfsugcaAfggacAfcUfugauususu | 121 |
| 18436 | P29 | T106 | asUfsugcaAfggacAfcUfugauuscsu | 122 |
| 8395 | P19 | T107 | asUfsgUfcAfuAfGfaggaCfcAfagacususu | 123 |
| 8401 | P9 | T107 | asUfsgucaUfagagGfaCfcaagacususu | 124 |
| 11344 | P24 | T107 | asUfsgUfcAfuagaggaCfcAfagacususu | 125 |
| 4995 | P1 | T108 | {Phos}asGfsuUfgGfUfgcuuCfuUfcagaasusu | 126 |
| 6182 | P2 | T108 | {Phos}asGfsuUfgGfUfgcuuCfuUfcagaasusu | 126 |
| 6150 | P7 | T108 | {Phos}asGfsuUfgGfUfgcuuCfuUfcagaaggsusu | 127 |

In an initial experiment, RNAi constructs having the same nucleotide sequence were synthesized to have either the CM1 control chemical modification pattern (duplex no. 3632) or the P1 chemical modification pattern (duplex no. 3635). In vivo efficacy of the two constructs was evaluated in a double transgenic mouse model, which express a fully functional human Lp(a) particle with serum baseline Lp(a) levels of about 50-60 mg/dL on average. Lp(a) is a low-density lipoprotein consisting of an LDL particle and the glycoprotein apolipoprotein (a) (apo(a)), which is linked to the apolipoprotein B of the LDL particle by a disulfide bond. Apo(a) is encoded by the LPA gene and changes in serum Lp(a) levels reflect changes in expression of the LPA gene. The double transgenic mice were generated by crossing transgenic mice expressing human apo(a) from a yeast artificial chromosome (YAC) containing the full human LPA gene (Frazer et al., Nature Genetics, Vol. 9: 424-431, 1995) with transgenic mice expressing human apoB-100 (Linton et al., J. Clin. Invest., Vol. 92: 3029-3037, 1993). The LPA RNAi constructs were administered as a single subcutaneous injection at a dose of 0.5 mg/kg. Serum samples were taken prior to injection and then post injection at day 14 and day 28. Lp(a) concentrations were measured in the serum using an Lp(a) ELISA assay (Cat. #10-1106-01, Mercodia AB, Uppsala, Sweden). A percentage change in Lp(a) level for each animal at a particular time point was calculated based on that animal's baseline Lp(a) level. The results are shown in FIG. 7. At two weeks after injection, although not statistically significant, administration of duplex no. 3635, which had the P1 modification pattern, resulted in a greater average decrease in serum Lp(a) levels (−49%) as compared to duplex no. 3632 (−35%), which had the control CM1 modification pattern.

In a second series of experiments, LPA RNAi constructs targeting distinct areas of the LPA mRNA from those in the first set of experiments were synthesized with the P1 chemical modification pattern or a variation of that pattern. The RNAi constructs with the new patterns were evaluated in the double transgenic mouse model for improvements in both magnitude and duration of suppression of LPA gene expression in vivo. Specifically, LPA RNAi constructs from three different sequence families having the P1 modification pattern or one of the pattern variants (e.g. P2, P4, P6 or P7 chemical modification patterns) were administered subcutaneously to the double transgenic mice described above at a dose of 2 mg/kg. Serum Lp(a) levels were measured in the animals prior to injection to obtain baseline levels and at weeks 1, 2, and 4 following administration of the LPA RNAi constructs. Results of this set of experiments are shown in Table 6 below. Across the three sequence families, RNAi constructs having the P2, P4, P6, or P7 modification pattern resulted in a greater reduction and duration of suppression of Lp(a) serum levels as compared to RNAi constructs having the P1 modification pattern. RNAi constructs having the P6 or P7 chemical modification patterns resulted in greater than 80% reduction of serum Lp(a) levels up to 4 weeks after a single subcutaneous injection of 2 mg/kg.

TABLE 6
In Vivo Efficacy of LPA RNAi Constructs

| | | | Percent Change in Serum Lp(a) from | | |
|---|---|---|---|---|---|
| | | Sequence | Baseline | | |
| Duplex | Pattern | Family | (mean ± SEM) | | |
| No. | Designation | Designation | Week 1 | Week 2 | Week 4 |
| 4973 | P1 | T102 | −66 ± 6% | −73 ± 7% | 16 ± 21% |
| 6248 | P4 | T102 | −73 ± 12% | −78 ± 10% | −37 ± 15% |
| 4601 | P1 | T103 | −92 ± 2% | −95 ± 1% | −76 ± 3% |
| 6247 | P6 | T103 | −93 ± 2% | −95 ± 1% | −86 ± 1% |
| 4995 | P1 | T108 | −70 ± 12% | −70 ± 12% | 14 ± 33% |
| 6182 | P2 | T108 | −87 ± 1% | −82 ± 5% | −45 ± 7% |
| 6150 | P7 | T108 | −92 ± 2% | −93 ± 2% | −83 ± 1% |

Next, alternative variations of the chemical modification patterns were designed and evaluated for in vivo efficacy in the double transgenic mouse model. The variations of the chemical modifications pattern were applied to RNAi constructs with sequences from five different sequence families. The sequences of the sense and antisense strands for each of the RNAi constructs are provided in Table 5 and the modification patterns are shown schematically in FIG. 1. The RNAi constructs were administered subcutaneously to double transgenic mice expressing human Lp(a) particles at a dose of 1 mg/kg. Serum Lp(a) levels were measured in the animals prior to injection to obtain baseline levels and at weeks 2, 3, and 4 following administration of the LPA RNAi constructs. The results are shown in Table 7 below. Several of the pattern variations, such as P9, P19, P22, P24, P27, P28, and P29, resulted in reduced Lp(a) serum levels by greater than 50% at four weeks following a single subcutaneous injection of 1 mg/kg. RNAi constructs having the P27 chemical modification pattern were particularly effective in suppressing Lp(a) serum levels as these constructs produced a sustained reduction of about 75% of Lp(a) levels at four weeks following a single injection.

TABLE 7
In Vivo Efficacy of LPA RNAi Constructs with
Alternative Chemical Modification Patterns

| | | Sequence | Average Percent Change in Serum Lp(a) | | |
|---|---|---|---|---|---|
| Duplex | Pattern | Family | from Baseline | | |
| No. | Designation | Designation | Week 2 | Week 3 | Week 4 |
| 7934 | P19 | T102 | −77% | −76% | −28% |
| 10927 | P27 | T102 | −92% | −85% | −77% |
| 11351 | P28 | T102 | −85% | −89% | −68% |
| 8336 | P10 | T104 | −39% | −59% | −16% |
| 11318 | P22 | T104 | −64% | −81% | −64% |
| 11313 | P25 | T104 | −51% | −54% | +3 |
| 11372 | P19 | T105 | −72% | −75% | −13% |
| 17183 | P25 | T105 | −56% | −37% | −16% |
| 18444 | P30 | T105 | −75% | −70% | −44% |
| 11580 | P27 | T106 | −86% | −78% | −74% |
| 18436 | P29 | T106 | −87% | −80% | −63% |
| 8401 | P9 | T107 | −90% | −82% | −58% |
| 8395 | P19 | T107 | −79% | −84% | −59% |
| 11344 | P24 | T107 | −87% | −75% | −67% |

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed:

1. An RNAi construct that inhibits expression of a target gene sequence, comprising a sense strand and an antisense strand, wherein the antisense strand comprises a sequence that is complementary to the target gene sequence and the sense strand comprises a sequence that is sufficiently complementary to the sequence of the antisense strand to form a duplex region, wherein the RNAi construct comprises a structure represented by Formula (A):
(A)
5′-(N_{A})ₓ N_{L} N_{L} N_{L} N_{L} N_{L} N_{L} N_{F} N_{L} N_{F} N_{F} N_{F} N_{F} N_{L} N_{L}
N_{M} N_{L} N_{M} N_{L} N_{T} (n)_{y}-3′
3′-(N_{B})_{z} N_{L} N_{L} N_{L} N_{L} N_{L} N_{F} N_{L} N_{M} N_{L} N_{M} N_{L} N_{L} N_{F} N_{M}
N_{L} N_{M} N_{L} N_{F} N_{L}-5′
wherein:
the top strand listed in the 5′ to 3′ direction is the sense strand and the bottom strand listed in the 3′ to 5′ direction is the antisense strand;
each N_{F} represents a 2′-fluoro modified nucleotide;
each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a bicyclic nucleic acid (BNA), and a deoxyribonucleotide;
each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
x is an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;
y is an integer from 0 to 4, provided that when y is 1, 2, 3, or 4, one or more n nucleotides are modified or unmodified overhang nucleotides that do not base pair with nucleotides in the antisense strand; and
z is an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

2. The RNAi construct of claim 1, wherein the sense strand and the antisense strand are each independently 19 to 30 nucleotides in length.

3. The RNAi construct of claim 1, wherein the sense strand and the antisense strand are each independently 19 to 25 nucleotides in length.

4. The RNAi construct of claim 1, wherein x is 0, y is 2, and z is 2.

5. The RNAi construct of claim 1, wherein x is 1 and N_{A} is an inverted abasic nucleotide, y is 2, and z is 2.

6. The RNAi construct of claim 1, wherein x is 2, y is 0, and z is 4.

7. The RNAi construct of claim 1, wherein x is 2, y is 0, and z is 2.

8. The RNAi construct of claim 1, wherein x is 3 and the N_{A} at the 5′ end is an inverted abasic nucleotide, y is 0, and z is 4.

9. The RNAi construct of claim 1, wherein x is 0, y is 0, and z is 2.

10. The RNAi construct of claim 1, wherein x is 1 and N_{A} is an inverted abasic nucleotide, y is 0, and z is 2.

11. The RNAi construct of any one of claims 1 to 10, wherein N_{T} is an inverted abasic nucleotide, an inverted deoxyribonucleotide, or a 2′-O-methyl modified nucleotide.

12. The RNAi construct of any one of claims 1 to 11, wherein each N_{L} in both the sense and antisense strands is a 2′-O-methyl modified nucleotide.

13. The RNAi construct of any one of claims 1 to 12, wherein the N_{M} at positions 4 and 12 in the antisense strand counting from the 5′ end are each a 2′-fluoro modified nucleotide.

14. The RNAi construct of claim 13, wherein the N_{M} at position 6 in the antisense strand counting from the 5′ end is a 2′-fluoro modified nucleotide.

15. The RNAi construct of claim 14, wherein the N_{M} at position 10 in the antisense strand counting from the 5′ end is a 2′-fluoro modified nucleotide.

16. The RNAi construct of any one of claims 1 to 12, wherein the N_{M} at positions 10 and 12 in the antisense strand counting from the 5′ end are each a 2′-fluoro modified nucleotide.

17. The RNAi construct of claim 16, wherein the N_{M} at position 4 in the antisense strand counting from the 5′ end is a 2′-fluoro modified nucleotide.

18. The RNAi construct of any one of claims 1 to 12, wherein the N_{M} at positions 4, 6, and 10 in the antisense strand counting from the 5′ end are each a 2′-O-methyl modified nucleotide, and the N_{M} at position 12 in the antisense strand counting from the 5′ end is a 2′-fluoro modified nucleotide.

19. The RNAi construct of any one of claims 1 to 12, wherein each N_{M} in both the sense and antisense strands is a 2′-O-methyl modified nucleotide.

20. The RNAi construct of any one of claims 1 to 18, wherein each N_{M} in the sense strand is a 2′-O-methyl modified nucleotide.

21. The RNAi construct of any one of claims 1 to 18, wherein each N_{M} in the sense strand is a 2′-fluoro modified nucleotide.

22. An RNAi construct that inhibits expression of a target gene sequence, comprising a sense strand and an antisense strand, wherein the antisense strand comprises a sequence that is complementary to the target gene sequence and the sense strand comprises a sequence that is sufficiently complementary to the sequence of the antisense strand to form a duplex region, wherein the RNAi construct comprises a structure represented by Formula (B):
(B)
5′-(N_{A})ₓ N_{L} N_{L} N_{L} N_{L} N_{L} N_{L} N_{F} N_{L} N_{F} N_{F} N_{F} N_{F} N_{L} N_{L}
N_{L} N_{L} N_{L} N_{L} N_{T} (n)_{y}-3′
3′-(N_{B})_{z} N_{L} N_{L} N_{L} N_{L} N_{L} N_{F} N_{L} N_{F} N_{L} N_{L} N_{L} N_{L} N_{F} N_{F}
N_{L} N_{F} N_{L} N_{F} N_{L}-5′
wherein:
the top strand listed in the 5′ to 3′ direction is the sense strand and the bottom strand listed in the 3′ to 5′ direction is the antisense strand;
each N_{F} represents a 2′-fluoro modified nucleotide;
each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
x is an integer from 0 to 4, provided that when x is 1, 2, 3, or 4, one or more of the NA nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;
y is an integer from 0 to 4, provided that when y is 1, 2, 3, or 4, one or more n nucleotides are modified or unmodified overhang nucleotides that do not base pair with nucleotides in the antisense strand; and
z is an integer from 0 to 4, provided that when z is 1, 2, 3, or 4, one or more of the NB nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

23. The RNAi construct of claim 22, wherein x is 0, y is 2, and z is 2.

24. The RNAi construct of claim 22, wherein x is 0, y is 0, and z is 2.

25. The RNAi construct of claim 22, wherein x is 1 and N_{A} is an inverted abasic nucleotide, y is 2, and z is 2.

26. The RNAi construct of claim 22, wherein x is 2, y is 0, and z is 4.

27. The RNAi construct of claim 22, wherein x is 3 and the N_{A} at the 5′ end is an inverted abasic nucleotide, y is 0, and z is 4.

28. The RNAi construct of any one of claims 22 to 27, wherein N_{T} is an inverted abasic nucleotide, an inverted deoxyribonucleotide, or a 2′-O-methyl modified nucleotide.

29. The RNAi construct of any one of claims 22 to 28, wherein each N_{L} in both the sense and antisense strands is a 2′-O-methyl modified nucleotide.

30. An RNAi construct that inhibits expression of a target gene sequence, comprising a sense strand and an antisense strand, wherein the antisense strand comprises a sequence that is complementary to the target gene sequence and the sense strand comprises a sequence that is sufficiently complementary to the sequence of the antisense strand to form a duplex region, wherein the RNAi construct comprises a structure represented by Formula (C):
(C)
5′-(AB)ₓ N_{L} N_{L} N_{L} N_{L} N_{L} N_{L} N_{L} N_{L} N_{F} N_{L} N_{F} N_{F} N_{F} N_{F}
N_{L} N_{L} N_{M} N_{L} N_{M} N_{L} N_{T}-3′
3′-N_{L} N_{L} N_{L} N_{L} N_{L} N_{L} N_{L} N_{L} N_{L} N_{F} N_{L} N_{F} N_{L} N_{L} N_{L} N_{L}
N_{F} N_{L} N_{L} N_{M} N_{L} N_{F} N_{L}-5′
wherein:
the top strand listed in the 5′ to 3′ direction is the sense strand and the bottom strand listed in the 3′ to 5′ direction is the antisense strand;
each N_{F} represents a 2′-fluoro modified nucleotide;
each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, BNA, and a deoxyribonucleotide;
N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide; and
x is 0 or 1 and Ab is an inverted abasic nucleotide.

31. The RNAi construct of claim 30, wherein each N_{M} in both the sense and antisense strands is a 2′-O-methyl modified nucleotide.

32. The RNAi construct of claim 31, wherein N_{T} is an inverted abasic nucleotide or inverted deoxyribonucleotide and x is 0.

33. The RNAi construct of claim 31, wherein N_{T} is a 2′-O-methyl modified nucleotide and x is 1.

34. The RNAi construct of claim 30, wherein the N_{M} in the antisense strand is a 2′-fluoro modified nucleotide.

35. The RNAi construct of claim 34, wherein each N_{M} in the sense strand is a 2′-O-methyl modified nucleotide.

36. The RNAi construct of claim 34, wherein each N_{M} in the sense strand is a 2′-fluoro modified nucleotide.

37. The RNAi construct of any one of claims 34 to 36, wherein N_{T} is an inverted abasic nucleotide or inverted deoxyribonucleotide and x is 0.

38. The RNAi construct of any one of claims 30 to 37, wherein each Ni, in both the sense and antisense strands is a 2′-O-methyl modified nucleotide.

39. An RNAi construct that inhibits expression of a target gene sequence, comprising a sense strand and an antisense strand, wherein the antisense strand comprises a sequence that is complementary to the target gene sequence and the sense strand comprises a sequence that is sufficiently complementary to the sequence of the antisense strand to form a duplex region, wherein the RNAi construct comprises a structure represented by Formula (D):
(D)
5′-(N_{A})ₓ N_{L} N_{L} N_{L} N_{L} N_{M} N_{L} N_{F} N_{F} N_{F} N_{F} N_{L} N_{L} N_{L} N_{L}
N_{L} N_{L} N_{L} N_{L} N_{T}(n)_{y}-3′
3′-(N_{B})_{z} N_{L} N_{L} N_{L} N_{M} N_{L} N_{F} N_{L} N_{M} N_{L} N_{L} N_{M} N_{M} N_{M} N_{M}
N_{L} N_{M} N_{L} N_{F} N_{L}-5′
wherein:
the top strand listed in the 5′ to 3′ direction is the sense strand and the bottom strand listed in the 3′ to 5′ direction is the antisense strand;
each N_{F} represents a 2′-fluoro modified nucleotide;
each N_{M} independently represents a modified nucleotide selected from a 2′-fluoro modified nucleotide, a 2′-O-methyl modified nucleotide, a 2′-O-methoxyethyl modified nucleotide, a 2′-O-alkyl modified nucleotide, a 2′-O-allyl modified nucleotide, a bicyclic nucleic acid (BNA), and a deoxyribonucleotide;
each N_{L} independently represents a modified nucleotide selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
N_{T} represents a modified nucleotide selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide;
x is an integer from 0 to 4, provided that when x is 1, 2, 3, or 4, one or more of the N_{A} nucleotides is a modified nucleotide independently selected from an abasic nucleotide, an inverted abasic nucleotide, an inverted deoxyribonucleotide, a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{A} nucleotides can be complementary to nucleotides in the antisense strand;
y is an integer from 0 to 4, provided that when y is 1, 2, 3, or 4, one or more n nucleotides are modified or unmodified overhang nucleotides that do not base pair with nucleotides in the antisense strand; and
z is an integer from 0 to 4, provided that when z is 1, 2, 3, or 4, one or more of the N_{B} nucleotides is a modified nucleotide independently selected from a 2′-O-methyl modified nucleotide, a 2′-O-methoxyethyl modified nucleotide, a 2′-O-alkyl modified nucleotide, a 2′-O-allyl modified nucleotide, a BNA, and a deoxyribonucleotide, and one or more of the N_{B} nucleotides can be complementary to N_{A} nucleotides when present in the sense strand or can be overhang nucleotides that do not base pair with nucleotides in the sense strand.

40. The RNAi construct of claim 39, wherein the sense strand and the antisense strand are each independently 19 to 30 nucleotides in length.

41. The RNAi construct of claim 39, wherein the sense strand and the antisense strand are each independently 19 to 25 nucleotides in length.

42. The RNAi construct of claim 39, wherein x is 2, y is 0, and z is 4.

43. The RNAi construct of claim 39, wherein x is 1 and N_{A} is an inverted abasic nucleotide, y is 2, and z is 2.

44. The RNAi construct of claim 39, wherein x is 1 and N_{A} is an inverted abasic nucleotide, y is 0, and z is 2.

45. The RNAi construct of claim 39, wherein x is 0, y is 0, and z is 2.

46. The RNAi construct of claim 39, wherein x is 2, y is 0, and z is 2.

47. The RNAi construct of any one of claims 39 to 46, wherein N_{T} is an inverted abasic nucleotide, an inverted deoxyribonucleotide, or a 2′-O-methyl modified nucleotide.

48. The RNAi construct of any one of claims 39 to 47, wherein each N_{L} in both the sense and antisense strands is a 2′-O-methyl modified nucleotide.

49. The RNAi construct of any one of claims 39 to 48, wherein the N_{M} at positions 4, 6, 8, 9, and 16 in the antisense strand counting from the 5′ end are each a 2′-fluoro modified nucleotide and the N_{M} at positions 7 and 12 in the antisense strand counting from the 5′ end are each a 2′-O-methyl modified nucleotide.

50. The RNAi construct of any one of claims 39 to 48, wherein the N_{M} at positions 4, 6, 8, 9, and 16 in the antisense strand counting from the 5′ end are each a 2′-O-methyl modified nucleotide and the N_{M} at positions 7 and 12 in the antisense strand counting from the 5′ end are each a 2′-fluoro modified nucleotide.

51. The RNAi construct of any one of claims 39 to 48, wherein the N_{M} at positions 4, 6, 8, 9, and 12 in the antisense strand counting from the 5′ end are each a 2′-O-methyl modified nucleotide and the N_{M} at positions 7 and 16 in the antisense strand counting from the 5′ end are each a 2′-fluoro modified nucleotide.

52. The RNAi construct of any one of claims 39 to 48, wherein the N_{M} at positions 7, 8, 9, and 12 in the antisense strand counting from the 5′ end are each a 2′-O-methyl modified nucleotide and the N_{M} at positions 4, 6, and 16 in the antisense strand counting from the 5′ end are each a 2′-fluoro modified nucleotide.

53. The RNAi construct of any one of claims 39 to 52, wherein the N_{M} in the sense strand is a 2′-fluoro modified nucleotide.

54. The RNAi construct of any one of claims 39 to 52, wherein the N_{M} in the sense strand is a 2′-O-methyl modified nucleotide.

55. The RNAi construct of any one of claims 1 to 54, wherein the sense strand, the antisense strand, or both the sense and antisense strands comprise one or more phosphorothioate internucleotide linkages.

56. The RNAi construct of claim 55, wherein the antisense strand comprises two consecutive phosphorothioate internucleotide linkages between the terminal nucleotides at both the 3′ and 5′ ends.

57. The RNAi construct of claim 55 or 56, wherein the sense strand comprises a single phosphorothioate internucleotide linkage between the terminal nucleotides at the 3′ end.

58. The RNAi construct of claim 55 or 56, wherein the sense strand comprises two consecutive phosphorothioate internucleotide linkages between the terminal nucleotides at the 3′ end.

59. The RNAi construct of any one of claims 1 to 58, wherein the RNAi construct further comprises a ligand.

60. The RNAi construct of claim 59, wherein the ligand comprises a cholesterol moiety, a vitamin, a steroid, a bile acid, a folate moiety, a fatty acid, a carbohydrate, a glycoside, or antibody or antigen-binding fragment thereof.

61. The RNAi construct of claim 59, wherein the ligand targets delivery of the RNAi construct to hepatocytes.

62. The RNAi construct of claim 59, wherein the ligand comprises galactose, galactosamine, or N-acetyl-galactosamine.

63. The RNAi construct of claim 62, wherein the ligand comprises a multivalent galactose moiety or multivalent N-acetyl-galactosamine moiety.

64. The RNAi construct of claim 63, wherein the multivalent galactose moiety or multivalent N-acetyl-galactosamine moiety is trivalent or tetravalent.

65. The RNAi construct of any one of claims 59 to 64, wherein the ligand is covalently attached to the sense strand optionally through a linker.

66. The RNAi construct of claim 65, wherein the ligand is covalently attached to the 5′ end of the sense strand.

67. A pharmaceutical composition comprising the RNAi construct of any one of claims 1 to 66 and a pharmaceutically acceptable carrier or excipient.

68. A method for inhibiting the expression of a target gene in a cell comprising contacting the cell with the RNAi construct of any one of claims 1 to 66.

69. The method of claim 68, wherein the cell is in vivo.

70. A method for inhibiting the expression of a target gene in a subject comprising administering to the subject the RNAi construct of any one of claims 1 to 66.

71. The method of claim 70, wherein the RNAi construct is administered to the subject via a parenteral route of administration.


SEQUENCE LISTING (127 sequences)

SEQ ID NO: 1 - RNA, 21 nt, Artificial Sequence
  Synthetic polynucleotide
cggccaauguccaccagcuuu

SEQ ID NO: 2 - RNA, 21 nt, Artificial Sequence
  Synthetic polynucleotide
cggccaauguccaccagcuuu

SEQ ID NO: 3 - RNA, 21 nt, Artificial Sequence
  Synthetic polynucleotide
gguccagccugaacuucuuuu

SEQ ID NO: 4 - RNA, 21 nt, Artificial Sequence
  Synthetic polynucleotide
gguccagccugaacuucuuuu

SEQ ID NO: 5 - RNA, 21 nt, Artificial Sequence
  Synthetic polynucleotide
gcuucaugcccuucuacaguu

SEQ ID NO: 6 - RNA, 21 nt, Artificial Sequence
  Synthetic polynucleotide
gcuucaugcccuucuacaguu

SEQ ID NO: 7 - RNA, 21 nt, Artificial Sequence
  Synthetic polynucleotide
gcggcuuccugggcuucuauu

SEQ ID NO: 8 - RNA, 21 nt, Artificial Sequence
  Synthetic polynucleotide
gcggcuuccugggcuucuauu

SEQ ID NO: 9 - RNA, 21 nt, Artificial Sequence
  Synthetic polynucleotide
gugacaacguacccuucauuu

SEQ ID NO: 10 - RNA, 21 nt, Artificial Sequence
  Synthetic polynucleotide
gugacaacguacccuucauuu

SEQ ID NO: 11 - RNA, 21 nt, Artificial Sequence
  Synthetic polynucleotide
gguauguuccugcuucauguu

SEQ ID NO: 12 - RNA, 21 nt, Artificial Sequence
  Synthetic polynucleotide
gguauguuccugcuucauguu

SEQ ID NO: 13 - RNA, 21 nt, Artificial Sequence
  Synthetic polynucleotide
guauguuccugcuucaugcuu

SEQ ID NO: 14 - RNA, 21 nt, Artificial Sequence
  Synthetic polynucleotide
guauguuccugcuucaugcuu

SEQ ID NO: 15 - RNA, 21 nt, Artificial Sequence
  Synthetic polynucleotide
uguuccugcuucaugcccuuu

SEQ ID NO: 16 - RNA, 21 nt, Artificial Sequence
  Synthetic polynucleotide
uguuccugcuucaugcccuuu

SEQ ID NO: 17 - RNA, 21 nt, Artificial Sequence
  Synthetic polynucleotide
guuccugcuucaugcccuuuu

SEQ ID NO: 18 - RNA, 21 nt, Artificial Sequence
  Synthetic polynucleotide
guuccugcuucaugcccuuuu

SEQ ID NO: 19 - RNA, 21 nt, Artificial Sequence
  Synthetic polynucleotide
ccugcuucaugcccuucuauu

SEQ ID NO: 20 - RNA, 21 nt, Artificial Sequence
  Synthetic polynucleotide
ccugcuucaugcccuucuauu

SEQ ID NO: 21 - RNA, 21 nt, Artificial Sequence
  Synthetic polynucleotide
cuucaugcccuucuacaguuu

SEQ ID NO: 22 - RNA, 21 nt, Artificial Sequence
  Synthetic polynucleotide
cuucaugcccuucuacaguuu

SEQ ID NO: 23 - RNA, 21 nt, Artificial Sequence
  Synthetic polynucleotide
uucaugcccuucuacaguguu

SEQ ID NO: 24 - RNA, 21 nt, Artificial Sequence
  Synthetic polynucleotide
uucaugcccuucuacaguguu

SEQ ID NO: 25 - RNA, 21 nt, Artificial Sequence
  Synthetic polynucleotide
augcccuucuacaguggccuu

SEQ ID NO: 26 - RNA, 21 nt, Artificial Sequence
  Synthetic polynucleotide
augcccuucuacaguggccuu

SEQ ID NO: 27 - RNA, 21 nt, Artificial Sequence
  Synthetic polynucleotide
gcuucaugcccuucuacauuu

SEQ ID NO: 28 - RNA, 21 nt, Artificial Sequence
  Synthetic polynucleotide
gcuucaugcccuucuacauuu

SEQ ID NO: 29 - RNA, 20 nt, Artificial Sequence
  Synthetic polynucleotide
cugcuucaugcccuucuaca

SEQ ID NO: 30 - RNA, 21 nt, Artificial Sequence
  Synthetic polynucleotide
cugcuucaugcccuucuacau

SEQ ID NO: 31 - RNA, 20 nt, Artificial Sequence
  Synthetic polynucleotide
cugcuucaugccuuucuaca

SEQ ID NO: 32 - RNA, 20 nt, Artificial Sequence
  Synthetic polynucleotide
cugcuucaugccuuucuaca

SEQ ID NO: 33 - RNA, 20 nt, Artificial Sequence
  Synthetic polynucleotide
ugcuucaugccuuucuacag

SEQ ID NO: 34 - RNA, 20 nt, Artificial Sequence
  Synthetic polynucleotide
ugcuucaugccuuucuacag

SEQ ID NO: 35 - RNA, 20 nt, Artificial Sequence
  Synthetic polynucleotide
cugcuucaugccuuucuaca

SEQ ID NO: 36 - RNA, 21 nt, Artificial Sequence
  Synthetic polynucleotide
cugcuucaugccuuucuacau

SEQ ID NO: 37 - RNA, 21 nt, Artificial Sequence
  Synthetic polynucleotide
cugcuucaugccuuucuacau

SEQ ID NO: 38 - RNA, 20 nt, Artificial Sequence
  Synthetic polynucleotide
cugcuucaugccuuucuaca

SEQ ID NO: 39 - RNA, 20 nt, Artificial Sequence
  Synthetic polynucleotide
ugcuucaugccuuucuacag

SEQ ID NO: 40 - RNA, 21 nt, Artificial Sequence
  Synthetic polynucleotide
agcugguggacauuggccguu

SEQ ID NO: 41 - RNA, 21 nt, Artificial Sequence
  Synthetic polynucleotide
agcugguggacauuggccguu

SEQ ID NO: 42 - RNA, 21 nt, Artificial Sequence
  Synthetic polynucleotide
aagaaguucaggcuggaccuu

SEQ ID NO: 43 - RNA, 21 nt, Artificial Sequence
  Synthetic polynucleotide
aagaaguucaggcuggaccuu

SEQ ID NO: 44 - RNA, 21 nt, Artificial Sequence
  Synthetic polynucleotide
cuguagaagggcaugaagcuu

SEQ ID NO: 45 - RNA, 21 nt, Artificial Sequence
  Synthetic polynucleotide
cuguagaagggcaugaagcuu

SEQ ID NO: 46 - RNA, 21 nt, Artificial Sequence
  Synthetic polynucleotide
uagaagcccaggaagccgcuu

SEQ ID NO: 47 - RNA, 21 nt, Artificial Sequence
  Synthetic polynucleotide
uagaagcccaggaagccgcuu

SEQ ID NO: 48 - RNA, 21 nt, Artificial Sequence
  Synthetic polynucleotide
augaaggguacguugucacuu

SEQ ID NO: 49 - RNA, 21 nt, Artificial Sequence
  Synthetic polynucleotide
augaaggguacguugucacuu

SEQ ID NO: 50 - RNA, 21 nt, Artificial Sequence
  Synthetic polynucleotide
caugaagcaggaacauaccuu

SEQ ID NO: 51 - RNA, 21 nt, Artificial Sequence
  Synthetic polynucleotide
caugaagcaggaacauaccuu

SEQ ID NO: 52 - RNA, 21 nt, Artificial Sequence
  Synthetic polynucleotide
gcaugaagcaggaacauacuu

SEQ ID NO: 53 - RNA, 21 nt, Artificial Sequence
  Synthetic polynucleotide
gcaugaagcaggaacauacuu

SEQ ID NO: 54 - RNA, 21 nt, Artificial Sequence
  Synthetic polynucleotide
agggcaugaagcaggaacauu

SEQ ID NO: 55 - RNA, 21 nt, Artificial Sequence
  Synthetic polynucleotide
agggcaugaagcaggaacauu

SEQ ID NO: 56 - RNA, 21 nt, Artificial Sequence
  Synthetic polynucleotide
aagggcaugaagcaggaacuu

SEQ ID NO: 57 - RNA, 21 nt, Artificial Sequence
  Synthetic polynucleotide
aagggcaugaagcaggaacuu

SEQ ID NO: 58 - RNA, 21 nt, Artificial Sequence
  Synthetic polynucleotide
uagaagggcaugaagcagguu

SEQ ID NO: 59 - RNA, 21 nt, Artificial Sequence
  Synthetic polynucleotide
uagaagggcaugaagcagguu

SEQ ID NO: 60 - RNA, 21 nt, Artificial Sequence
  Synthetic polynucleotide
acuguagaagggcaugaaguu

SEQ ID NO: 61 - RNA, 21 nt, Artificial Sequence
  Synthetic polynucleotide
acuguagaagggcaugaaguu

SEQ ID NO: 62 - RNA, 21 nt, Artificial Sequence
  Synthetic polynucleotide
cacuguagaagggcaugaauu

SEQ ID NO: 63 - RNA, 21 nt, Artificial Sequence
  Synthetic polynucleotide
cacuguagaagggcaugaauu

SEQ ID NO: 64 - RNA, 21 nt, Artificial Sequence
  Synthetic polynucleotide
ggccacuguagaagggcauuu

SEQ ID NO: 65 - RNA, 21 nt, Artificial Sequence
  Synthetic polynucleotide
ggccacuguagaagggcauuu

SEQ ID NO: 66 - RNA, 21 nt, Artificial Sequence
  Synthetic polynucleotide
ggccacuguagaagggcauuu

SEQ ID NO: 67 - RNA, 23 nt, Artificial Sequence
  Synthetic polynucleotide
auguagaagggcaugaagcaguu

SEQ ID NO: 68 - RNA, 23 nt, Artificial Sequence
  Synthetic polynucleotide
auguagaaaggcaugaagcaguu

SEQ ID NO: 69 - RNA, 23 nt, Artificial Sequence
  Synthetic polynucleotide
auguagaaaggcaugaagcaguu

SEQ ID NO: 70 - RNA, 23 nt, Artificial Sequence
  Synthetic polynucleotide
auguagaaaggcaugaagcaguu

SEQ ID NO: 71 - RNA, 23 nt, Artificial Sequence
  Synthetic polynucleotide
auguagaaaggcaugaagcaguu

SEQ ID NO: 72 - RNA, 23 nt, Artificial Sequence
  Synthetic polynucleotide
acuguagaaaggcaugaagcauu

SEQ ID NO: 73 - RNA, 23 nt, Artificial Sequence
  Synthetic polynucleotide
ucuguagaaaggcaugaagcauu

SEQ ID NO: 74 - RNA, 23 nt, Artificial Sequence
  Synthetic polynucleotide
auguagaaaggcaugaagcaguu

SEQ ID NO: 75 - RNA, 23 nt, Artificial Sequence
  Synthetic polynucleotide
auguagaaaggcaugaagcaguu

SEQ ID NO: 76 - RNA, 23 nt, Artificial Sequence
  Synthetic polynucleotide
auguagaaaggcaugaagcaguu

SEQ ID NO: 77 - RNA, 23 nt, Artificial Sequence
  Synthetic polynucleotide
auguagaaaggcaugaagcaguu

SEQ ID NO: 78 - RNA, 23 nt, Artificial Sequence
  Synthetic polynucleotide
auguagaaaggcaugaagcaguu

SEQ ID NO: 79 - RNA, 23 nt, Artificial Sequence
  Synthetic polynucleotide
auguagaaaggcaugaagcaguu

SEQ ID NO: 80 - RNA, 23 nt, Artificial Sequence
  Synthetic polynucleotide
auguagaaaggcaugaagcaguu

SEQ ID NO: 81 - RNA, 21 nt, Artificial Sequence
  Synthetic polynucleotide
gugggaagaaagaugaaguuu

SEQ ID NO: 82 - RNA, 21 nt, Artificial Sequence
  Synthetic polynucleotide
gugggaagaaagaugaaguuu

SEQ ID NO: 83 - RNA, 21 nt, Artificial Sequence
  Synthetic polynucleotide
acuucaucuuucuucccacuu

SEQ ID NO: 84 - RNA, 21 nt, Artificial Sequence
  Synthetic polynucleotide
acuucaucuuucuucccacuu

SEQ ID NO: 85 - RNA, 21 nt, Artificial Sequence
  Synthetic polynucleotide
gccccuuauuguuauacgauu

SEQ ID NO: 86 - RNA, 21 nt, Artificial Sequence
  Synthetic polynucleotide
gccccuuauuguuauacgauu

SEQ ID NO: 87 - RNA, 21 nt, Artificial Sequence
  Synthetic polynucleotide
acacaaugcucagacgcaguu

SEQ ID NO: 88 - RNA, 21 nt, Artificial Sequence
  Synthetic polynucleotide
ugacacaaugcucagacgcag

SEQ ID NO: 89 - RNA, 20 nt, Artificial Sequence
  Synthetic polynucleotide
ugacacaaugcucagacgca

SEQ ID NO: 90 - RNA, 20 nt, Artificial Sequence
  Synthetic polynucleotide
acacaaugcucagacgcaau

SEQ ID NO: 91 - RNA, 18 nt, Artificial Sequence
  Synthetic polynucleotide
acacaaugcucagacgca

SEQ ID NO: 92 - RNA, 21 nt, Artificial Sequence
  Synthetic polynucleotide
ccuagaggcuccuucugaauu

SEQ ID NO: 93 - RNA, 21 nt, Artificial Sequence
  Synthetic polynucleotide
agccuagaggcuccuucugaa

SEQ ID NO: 94 - RNA, 21 nt, Artificial Sequence
  Synthetic polynucleotide
uucgcccuugguguuacacca

SEQ ID NO: 95 - RNA, 21 nt, Artificial Sequence
  Synthetic polynucleotide
cgcccuugguguuacaccauu

SEQ ID NO: 96 - RNA, 19 nt, Artificial Sequence
  Synthetic polynucleotide
cgcccuugguguuacacca

SEQ ID NO: 97 - RNA, 20 nt, Artificial Sequence
  Synthetic polynucleotide
cagaaucaaguguccuugca

SEQ ID NO: 98 - RNA, 21 nt, Artificial Sequence
  Synthetic polynucleotide
gaaucaaguguccuugcaauu

SEQ ID NO: 99 - RNA, 20 nt, Artificial Sequence
  Synthetic polynucleotide
cagaaucaaguguccuugca

SEQ ID NO: 100 - RNA, 20 nt, Artificial Sequence
  Synthetic polynucleotide
aaucaaguguccuugcaauu

SEQ ID NO: 101 - RNA, 20 nt, Artificial Sequence
  Synthetic polynucleotide
agaaucaaguguccuugcaa

SEQ ID NO: 102 - RNA, 20 nt, Artificial Sequence
  Synthetic polynucleotide
agucuugguccucuaugaca

SEQ ID NO: 103 - RNA, 20 nt, Artificial Sequence
  Synthetic polynucleotide
agucuugguccucuaugaca

SEQ ID NO: 104 - RNA, 21 nt, Artificial Sequence
  Synthetic polynucleotide
uucugaagaagcaccaacuuu

SEQ ID NO: 105 - RNA, 21 nt, Artificial Sequence
  Synthetic polynucleotide
uucugaagaagcaccaacuuu

SEQ ID NO: 106 - RNA, 20 nt, Artificial Sequence
  Synthetic polynucleotide
ccuucugaagaagcaccaac

SEQ ID NO: 107 - RNA, 21 nt, Artificial Sequence
  Synthetic polynucleotide
ucguauaacaauaaggggcuu

SEQ ID NO: 108 - RNA, 21 nt, Artificial Sequence
  Synthetic polynucleotide
ucguauaacaauaaggggcuu

SEQ ID NO: 109 - RNA, 21 nt, Artificial Sequence
  Synthetic polynucleotide
cugcgucugagcauuguguuu

SEQ ID NO: 110 - RNA, 23 nt, Artificial Sequence
  Synthetic polynucleotide
cugcgucugagcauugugucauu

SEQ ID NO: 111 - RNA, 23 nt, Artificial Sequence
  Synthetic polynucleotide
uugcgucugagcauugugucauu

SEQ ID NO: 112 - RNA, 21 nt, Artificial Sequence
  Synthetic polynucleotide
uugcgucugagcauuguguuu

SEQ ID NO: 113 - RNA, 21 nt, Artificial Sequence
  Synthetic polynucleotide
uucagaaggagccucuagguu

SEQ ID NO: 114 - RNA, 23 nt, Artificial Sequence
  Synthetic polynucleotide
uucagaaggagccucuaggcuuu

SEQ ID NO: 115 - RNA, 23 nt, Artificial Sequence
  Synthetic polynucleotide
ugguguaacaccaagggcgaauu

SEQ ID NO: 116 - RNA, 21 nt, Artificial Sequence
  Synthetic polynucleotide
ugguguaacaccaagggcguu

SEQ ID NO: 117 - RNA, 21 nt, Artificial Sequence
  Synthetic polynucleotide
ugguguaacaccaagggcguu

SEQ ID NO: 118 - RNA, 23 nt, Artificial Sequence
  Synthetic polynucleotide
uugcaaggacacuugauucuguu

SEQ ID NO: 119 - RNA, 21 nt, Artificial Sequence
  Synthetic polynucleotide
uugcaaggacacuugauucuu

SEQ ID NO: 120 - RNA, 21 nt, Artificial Sequence
  Synthetic polynucleotide
uugcaaggacacuugauucug

SEQ ID NO: 121 - RNA, 21 nt, Artificial Sequence
  Synthetic polynucleotide
auugcaaggacacuugauuuu

SEQ ID NO: 122 - RNA, 21 nt, Artificial Sequence
  Synthetic polynucleotide
auugcaaggacacuugauucu

SEQ ID NO: 123 - RNA, 23 nt, Artificial Sequence
  Synthetic polynucleotide
augucauagaggaccaagacuuu

SEQ ID NO: 124 - RNA, 23 nt, Artificial Sequence
  Synthetic polynucleotide
augucauagaggaccaagacuuu

SEQ ID NO: 125 - RNA, 23 nt, Artificial Sequence
  Synthetic polynucleotide
augucauagaggaccaagacuuu

SEQ ID NO: 126 - RNA, 21 nt, Artificial Sequence
  Synthetic polynucleotide
aguuggugcuucuucagaauu

SEQ ID NO: 127 - RNA, 23 nt, Artificial Sequence
  Synthetic polynucleotide
aguuggugcuucuucagaagguu